SEQUENCE LISTING (156 sequences)

SEQ ID NO: 1 - protein, 229 aa, synthetic construct
  REGION 1..229: note = human EBI3 protein
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 2 - protein, 243 aa, synthetic construct
  REGION 1..243: note = human p28 protein
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 3 - protein, 636 aa, synthetic construct
  REGION 1..636: note = human WSX1
MRGGRGAPFWLWPLPKLALLPLLWVLFQRTRPQGSAGPLQCYGVGPLGDLNCSWEPLGDLGAPSELHLQSQKYRSNKTQTVAVAAGRSWVAIPREQLTMSDKLLVWGTKAGQPLWPPVFVNLETQMKPNAPRLGPDVDFSEDDPLEATVHWAPPTWPSHKVLICQFHYRRCQEAAWTLLEPELKTIPLTPVEIQDLELATGYKVYGRCRMEKEEDLWGEWSPILSFQTPPSAPKDVWVSGNLCGTPGGEEPLLLWKAPGPCVQVSYKVWFWVGGRELSPEGITCCCSLIPSGAEWARVSAVNATSWEPLTNLSLVCLDSASAPRSVAVSSIAGSTELLVTWQPGPGEPLEHVVDWARDGDPLEKLNWVRLPPGNLSALLPGNFTVGVPYRITVTAVSASGLASASSVWGFREELAPLVGPTLWRLQDAPPGTPAIAWGEVPRHQLRGHLTHYTLCAQSGTSPSVCMNVSGNTQSVTLPDLPWGPCELWVTASTIAGQGPPGPILRLHLPDNTLRWKVLPGILFLWGLFLLGCGLSLATSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLA

SEQ ID NO: 4 - protein, 918 aa, synthetic construct
  REGION 1..918: note = human gp130 protein
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 5 - protein, 232 aa, synthetic construct
  REGION 1..232: note = Human IgG1
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 229 aa, synthetic construct
  REGION 1..229: note = Human IgG4
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 229 aa, synthetic construct
  REGION 1..229: note = Human IgG4 (S228P)
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 229 aa, synthetic construct
  REGION 1..229: note = Human IgG4 (S228P / L235E)
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 8 aa, synthetic construct
  REGION 1..8: note = Heavy chain CDR1
GFTFSSYS

SEQ ID NO: 10 - protein, 8 aa, synthetic construct
  REGION 1..8: note = Heavy chain CDR2
ISSSSSYI

SEQ ID NO: 11 - protein, 19 aa, synthetic construct
  REGION 1..19: note = Heavy chain CDR3
ARDGGRTSYTATAHNWFDP

SEQ ID NO: 12 - protein, 9 aa, synthetic construct
  REGION 1..9: note = Heavy chain CDR1
FTFSSYSMN

SEQ ID NO: 13 - protein, 17 aa, synthetic construct
  REGION 1..17: note = Heavy chain CDR2
SISSSSSYIYYADSVKG

SEQ ID NO: 14 - protein, 19 aa, synthetic construct
  REGION 1..19: note = Heavy Chain CDR3
ARDGGRTSYTATAHNWFDP

SEQ ID NO: 15 - protein, 126 aa, synthetic construct
  REGION 1..126: note = Heavy Chain
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDGGRTSYTATAHNWFDPWGQGTLVTVSS

SEQ ID NO: 16 - DNA, 378 bp, synthetic construct
  misc_feature 1..378: note = DNA Heavy Chain
  source 1..378: mol_type = other DNA
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctca

SEQ ID NO: 17 - protein, 12 aa, synthetic construct
  REGION 1..12: note = Light Chain CDR1
QSVLFSSNNKNY

SEQ ID NO: 18 - skipped

SEQ ID NO: 19 - protein, 9 aa, synthetic construct
  REGION 1..9: note = Light Chain CDR3
QQHASAPPT

SEQ ID NO: 20 - protein, 17 aa, synthetic construct
  REGION 1..17: note = Light Chain CDR1
KSSQSVLFSSNNKNYLA

SEQ ID NO: 21 - protein, 7 aa, synthetic construct
  REGION 1..7: note = Light Chain CDR2
WASTRES

SEQ ID NO: 22 - protein, 9 aa, synthetic construct
  REGION 1..9: note = Light Chain CDR3
QQHASAPPT

SEQ ID NO: 23 - protein, 113 aa, synthetic construct
  REGION 1..113: note = Light Chain
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQHASAPPTFGGGTKVEIK

SEQ ID NO: 24 - DNA, 339 bp, synthetic construct
  misc_feature 1..339: note = DNA Light Chain
  source 1..339: mol_type = other DNA
gacatcgtgatgacccagtctccagactccctggctgtgtctctgggcgagagggccaccatcaactgcaagtccagccagagtgttttattcagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcagcacgccagtgcccctcctacttttggcggagggaccaaggttgagatcaaa

SEQ ID NO: 25 - protein, 456 aa, synthetic construct
  REGION 1..456: note = Heavy Chain
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDGGRTSYTATAHNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - DNA, 1368 bp, synthetic construct
  misc_feature 1..1368: note = DNA Heavy Chain
  source 1..1368: mol_type = other DNA
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 27 - protein, 220 aa, synthetic construct
  REGION 1..220: note = Light Chain
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQHASAPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - DNA, 660 bp, synthetic construct
  misc_feature 1..660: note = DNA Light Chain
  source 1..660: mol_type = other DNA
gacatcgtgatgacccagtctccagactccctggctgtgtctctgggcgagagggccaccatcaactgcaagtccagccagagtgttttattcagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcagcacgccagtgcccctcctacttttggcggagggaccaaggttgagatcaaacgtacggtggccgctccctccgtgttcatcttcccaccctccgacgagcagctgaagtccggcaccgcctccgtcgtgtgcctgctgaacaacttctaccctcgcgaggccaaagtgcagtggaaagtggacaacgccctgcagtccggcaactcccaggaatccgtcaccgagcaggactccaaggacagcacctactccctgtcctccaccctgaccctgtccaaggccgactacgagaagcacaaagtgtacgcctgcgaagtgacccaccagggcctgtccagccccgtgaccaagtccttcaaccggggcgagtgc

SEQ ID NO: 29 - protein, 452 aa, synthetic construct
  REGION 1..452: note = Heavy Chain
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDGGRTSYTATAHNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 30 - DNA, 1356 bp, synthetic construct
  misc_feature 1..1356: note = DNA Heavy Chain
  source 1..1356: mol_type = other DNA
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcttccaccaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgcccttcctgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggc

SEQ ID NO: 31 - protein, 8 aa, synthetic construct
  REGION 1..8: note = Heavy Chain CDR1
GFTFRSYG

SEQ ID NO: 32 - protein, 8 aa, synthetic construct
  REGION 1..8: note = Heavy chain CDR2
ISSSSSYI

SEQ ID NO: 33 - protein, 19 aa, synthetic construct
  REGION 1..19: note = Heavy Chain CDR3
ARDGGRTSYTATAHNWFDP

SEQ ID NO: 34 - protein, 9 aa, synthetic construct
  REGION 1..9: note = Heavy Chain CDR1
FTFRSYGMN

SEQ ID NO: 35 - protein, 17 aa, synthetic construct
  REGION 1..17: note = Heavy Chain CDR2
SISSSSSYIYYADSVKG

SEQ ID NO: 36 - protein, 19 aa, synthetic construct
  REGION 1..19: note = Heavy Chain CDR3
ARDGGRTSYTATAHNWFDP

SEQ ID NO: 37 - protein, 126 aa, synthetic construct
  REGION 1..126: note = Heavy Chain
EVQLVESGGGLVKPGGSLRLSCAASGFTFRSYGMNWVRQAPGKGLEWVSSISSSSSYIYYADSVKGRFTISRDNAKNSLYLQMNSLRAEDTAVYYCARDGGRTSYTATAHNWFDPWGQGTLVTVSS

SEQ ID NO: 38 - DNA, 378 bp, synthetic construct
  misc_feature 1..378: note = DNA Heavy Chain
  source 1..378: mol_type = other DNA
gaggtgcagctggtggagtctgggggaggcctggtcaagcctggggggtccctgagactctcctgtgcagcctctggattcaccttccggagctatgggatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctca

SEQ ID NO: 39 - protein, 12 aa, synthetic construct
  REGION 1..12: note = Light Chain CDR1
QSVLFSSNNKNY

SEQ ID NO: 40 - skipped

SEQ ID NO: 41 - protein, 9 aa, synthetic construct
  REGION 1..9: note = Light Chain CDR3
QQHASAPPT

SEQ ID NO: 42 - protein, 17 aa, synthetic construct
  REGION 1..17: note = Light Chain CDR1
KSSQSVLFSSNNKNYLA

SEQ ID NO: 43 - protein, 7 aa, synthetic construct
  REGION 1..7: note = Light Chain CDR2
WASTRES

SEQ ID NO: 44 - protein, 9 aa, synthetic construct
  REGION 1..9: note = Light Chain CDR3
QQHASAPPT

SEQ ID NO: 45 - protein, 113 aa, synthetic construct; identical to SEQ ID NO: 23
  REGION 1..113: note = Light Chain

SEQ ID NO: 46 - DNA, 339 bp, synthetic construct; identical to SEQ ID NO: 24
  misc_feature 1..339: note = DNA Light Chain
  source 1..339: mol_type = other DNA

SEQ ID NO: 47 - protein, 456 aa, synthetic construct
  REGION 1..456: note = Heavy Chain
EVQLVESGGGLVKPGGSLRLSCAASGFTFRSYGMNWVRQAPGKGLEWVSSISSSSSYIYYADSVKGRFTISRDNAKNSLYLQMNSLRAEDTAVYYCARDGGRTSYTATAHNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - DNA, 1368 bp, synthetic construct
  misc_feature 1..1368: note = DNA Heavy Chain
  source 1..1368: mol_type = other DNA
gaggtgcagctggtggagtctgggggaggcctggtcaagcctggggggtccctgagactctcctgtgcagcctctggattcaccttccggagctatgggatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 49 - protein, 220 aa, synthetic construct; identical to SEQ ID NO: 27
  REGION 1..220: note = Light Chain

SEQ ID NO: 50 - DNA, 660 bp, synthetic construct; identical to SEQ ID NO: 28
  misc_feature 1..660: note = DNA Light Chain
  source 1..660: mol_type = other DNA

SEQ ID NO: 51 - protein, 452 aa, synthetic construct
  REGION 1..452: note = Heavy Chain
EVQLVESGGGLVKPGGSLRLSCAASGFTFRSYGMNWVRQAPGKGLEWVSSISSSSSYIYYADSVKGRFTISRDNAKNSLYLQMNSLRAEDTAVYYCARDGGRTSYTATAHNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 52 - DNA, 1356 bp, synthetic construct
  misc_feature 1..1356: note = DNA Heavy Chain
  source 1..1356: mol_type = other DNA
gaggtgcagctggtggagtctgggggaggcctggtcaagcctggggggtccctgagactctcctgtgcagcctctggattcaccttccggagctatgggatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcttccaccaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgcccttcctgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggc

SEQ ID NO: 53 - protein, 8 aa, synthetic construct
  REGION 1..8: note = Heavy chain CDR1
GFTFSRTG

SEQ ID NO: 54 - protein, 8 aa, synthetic construct
  REGION 1..8: note = Heavy chain CDR2
ISSSSSYI

SEQ ID NO: 55 - protein, 19 aa, synthetic construct
  REGION 1..19: note = Heavy chain CDR3
ARDGGRTSYTATAHNWFDP

SEQ ID NO: 56 - protein, 9 aa, synthetic construct
  REGION 1..9: note = Heavy chain CDR1
FTFSRTGMN

SEQ ID NO: 57 - protein, 17 aa, synthetic construct
  REGION 1..17: note = Heavy chain CDR2
SISSSSSYIYYADSVKG

SEQ ID NO: 58 - protein, 19 aa, synthetic construct
  REGION 1..19: note = Heavy chain CDR3
ARDGGRTSYTATAHNWFDP

SEQ ID NO: 59 - protein, 126 aa, synthetic construct
  REGION 1..126: note = Heavy chain
EVQLVESGGGLVKPGGSLRLSCAASGFTFSRTGMNWVRQAPGKGLEWVSSISSSSSYIYYADSVKGRFTISRDNAKNSLYLQMNSLRAEDTAVYYCARDGGRTSYTATAHNWFDPWGQGTLVTVSS

SEQ ID NO: 60 - DNA, 378 bp, synthetic construct
  misc_feature 1..378: note = DNA Heavy chain
  source 1..378: mol_type = other DNA
gaggtgcagctggtggagtctgggggaggcctggtcaagcctggggggtccctgagactctcctgtgcagcctctggattcaccttcagtaggactgggatgaactgggtccgccaggctccagggaaggggctggaatgggtctcatccattagtagtagtagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctca

SEQ ID NO: 61 - protein, 12 aa, synthetic construct
  REGION 1..12: note = Light chain CDR1
QSVLFSSNNKNY

SEQ ID NO: 62 - skipped

SEQ ID NO: 63 - protein, 9 aa, synthetic construct
  REGION 1..9: note = Light chain CDR3
QQHASAPPT

SEQ ID NO: 64 - protein, 17 aa, synthetic construct
  REGION 1..17: note = Light chain CDR1
KSSQSVLFSSNNKNYLA

SEQ ID NO: 65 - protein, 7 aa, synthetic construct
  REGION 1..7: note = Light chain CDR2
WASTRES

SEQ ID NO: 66 - protein, 9 aa, synthetic construct
  REGION 1..9: note = Light chain CDR3
QQHASAPPT

SEQ ID NO: 67 - protein, 113 aa, synthetic construct; identical to SEQ ID NO: 23
  REGION 1..113: note = Light chain

SEQ ID NO: 68 - DNA, 339 bp, synthetic construct; identical to SEQ ID NO: 24
  misc_feature 1..339: note = DNA Light chain
  source 1..339: mol_type = other DNA

SEQ ID NO: 69 - protein, 456 aa, synthetic construct
  REGION 1..456: note = Heavy Chain
EVQLVESGGGLVKPGGSLRLSCAASGFTFSRTGMNWVRQAPGKGLEWVSSISSSSSYIYYADSVKGRFTISRDNAKNSLYLQMNSLRAEDTAVYYCARDGGRTSYTATAHNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - DNA, 1368 bp, synthetic construct
  misc_feature 1..1368: note = DNA Heavy Chain
  source 1..1368: mol_type = other DNA
gaggtgcagctggtggagtctgggggaggcctggtcaagcctggggggtccctgagactctcctgtgcagcctctggattcaccttcagtaggactgggatgaactgggtccgccaggctccagggaaggggctggaatgggtctcatccattagtagtagtagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 71 - protein, 220 aa, synthetic construct; identical to SEQ ID NO: 27
  REGION 1..220: note = Light Chain

SEQ ID NO: 72 - DNA, 660 bp, synthetic construct; identical to SEQ ID NO: 28
  misc_feature 1..660: note = DNA Light Chain
  source 1..660: mol_type = other DNA

SEQ ID NO: 73 - protein, 452 aa, synthetic construct
  REGION 1..452: note = Heavy Chain
EVQLVESGGGLVKPGGSLRLSCAASGFTFSRTGMNWVRQAPGKGLEWVSSISSSSSYIYYADSVKGRFTISRDNAKNSLYLQMNSLRAEDTAVYYCARDGGRTSYTATAHNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 74 - DNA, 1356 bp, synthetic construct
  misc_feature 1..1356: note = DNA Heavy Chain
  source 1..1356: mol_type = other DNA
gaggtgcagctggtggagtctgggggaggcctggtcaagcctggggggtccctgagactctcctgtgcagcctctggattcaccttcagtaggactgggatgaactgggtccgccaggctccagggaaggggctggaatgggtctcatccattagtagtagtagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcttccaccaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgcccttcctgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggc

SEQ ID NO: 75 - protein, 8 aa, synthetic construct
  REGION 1..8: note = Heavy chain CDR1
GFTFSRYG

SEQ ID NO: 76 - protein, 8 aa, synthetic construct
  REGION 1..8: note = Heavy chain CDR2
ISSSSAYI

SEQ ID NO: 77 - protein, 19 aa, synthetic construct
  REGION 1..19: note = Heavy chain CDR3
ARDGGRTSYTATAHNWFDP

SEQ ID NO: 78 - protein, 9 aa, synthetic construct
  REGION 1..9: note = Heavy chain CDR1
FTFSRYGMN

SEQ ID NO: 79 - protein, 17 aa, synthetic construct
  REGION 1..17: note = Heavy chain CDR2
SISSSSAYILYADSVKG

SEQ ID NO: 80 - protein, 19 aa, synthetic construct
  REGION 1..19: note = Heavy chain CDR3
ARDGGRTSYTATAHNWFDP

SEQ ID NO: 81 - protein, 126 aa, synthetic construct
  REGION 1..126: note = Heavy chain
EVQLVESGGGLVKPGGSLRLSCAASGFTFSRYGMNWVRQAPGKGLEWVSSISSSSAYILYADSVKGRFTISRDNAKNSLYLQMNSLRAEDTAVYYCARDGGRTSYTATAHNWFDPWGQGTLVTVSS

SEQ ID NO: 82 - DNA, 378 bp, synthetic construct
  misc_feature 1..378: note = DNA Heavy chain
  source 1..378: mol_type = other DNA
gaggtgcagctggtggagtctgggggaggcctggtcaagcctggggggtccctgagactctcctgtgcagcctctggattcaccttcagtaggtatgggatgaactgggtccgccaggctccagggaaggggctggagtgggtctcatccattagtagtagtagtgcttacatactgtacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctca

SEQ ID NO: 83 - protein, 12 aa, synthetic construct
  REGION 1..12: note = Light chain CDR1
QSVLFSSNNKNY

SEQ ID NO: 84 - skipped

SEQ ID NO: 85 - protein, 9 aa, synthetic construct
  REGION 1..9: note = Light chain CDR3
QQHASAPPT

SEQ ID NO: 86 - protein, 17 aa, synthetic construct
  REGION 1..17: note = Light chain CDR1
KSSQSVLFSSNNKNYLA

SEQ ID NO: 87 - protein, 7 aa, synthetic construct
  REGION 1..7: note = Light chain CDR2
WASTRES

SEQ ID NO: 88 - protein, 9 aa, synthetic construct
  REGION 1..9: note = Light chain CDR3
QQHASAPPT

SEQ ID NO: 89 - protein, 113 aa, synthetic construct; identical to SEQ ID NO: 23
  REGION 1..113: note = Light Chain

SEQ ID NO: 90 - DNA, 339 bp, synthetic construct; identical to SEQ ID NO: 24
  misc_feature 1..339: note = DNA Light Chain
  source 1..339: mol_type = other DNA

SEQ ID NO: 91 - protein, 456 aa, synthetic construct
  REGION 1..456: note = Heavy Chain
EVQLVESGGGLVKPGGSLRLSCAASGFTFSRYGMNWVRQAPGKGLEWVSSISSSSAYILYADSVKGRFTISRDNAKNSLYLQMNSLRAEDTAVYYCARDGGRTSYTATAHNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - DNA, 1368 bp, synthetic construct
  misc_feature 1..1368: note = DNA Heavy Chain
  source 1..1368: mol_type = other DNA
gaggtgcagctggtggagtctgggggaggcctggtcaagcctggggggtccctgagactctcctgtgcagcctctggattcaccttcagtaggtatgggatgaactgggtccgccaggctccagggaaggggctggagtgggtctcatccattagtagtagtagtgcttacatactgtacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 93 - protein, 220 aa, synthetic construct; identical to SEQ ID NO: 27
  REGION 1..220: note = Light Chain

SEQ ID NO: 94 - DNA, 660 bp, synthetic construct; identical to SEQ ID NO: 28
  misc_feature 1..660: note = DNA Light Chain
  source 1..660: mol_type = other DNA

SEQ ID NO: 95 - protein, 452 aa, synthetic construct
  REGION 1..452: note = Heavy CHain
EVQLVESGGGLVKPGGSLRLSCAASGFTFSRYGMNWVRQAPGKGLEWVSSISSSSAYILYADSVKGRFTISRDNAKNSLYLQMNSLRAEDTAVYYCARDGGRTSYTATAHNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 96 - DNA, 1356 bp, synthetic construct
  misc_feature 1..1356: note = DNA Heavy Chain
  source 1..1356: mol_type = other DNA
gaggtgcagctggtggagtctgggggaggcctggtcaagcctggggggtccctgagactctcctgtgcagcctctggattcaccttcagtaggtatgggatgaactgggtccgccaggctccagggaaggggctggagtgggtctcatccattagtagtagtagtgcttacatactgtacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcttccaccaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgcccttcctgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggc

SEQ ID NO: 97 - protein, 8 aa, synthetic construct
  REGION 1..8: note = Heavy chain CDR1
GFTFASYG

SEQ ID NO: 98 - protein, 8 aa, synthetic construct
  REGION 1..8: note = Heavy chain CDR2
ISSSSSYI

SEQ ID NO: 99 - protein, 19 aa, synthetic construct
  REGION 1..19: note = Heavy chain CDR3
ARDGGRTSYTATAHNWFDP

SEQ ID NO: 100 - protein, 9 aa, synthetic construct
  REGION 1..9: note = Heavy chain CDR1
FTFASYGMN

SEQ ID NO: 101 - protein, 17 aa, synthetic construct
  REGION 1..17: note = Heavy chain CDR2
SISSSSSYIYYADSVKG

SEQ ID NO: 102 - protein, 19 aa, synthetic construct
  REGION 1..19: note = Heavy chain CDR3
ARDGGRTSYTATAHNWFDP

SEQ ID NO: 103 - protein, 126 aa, synthetic construct
  REGION 1..126: note = Heavy chain
EVQLVESGGGLVKPGGSLRLSCAASGFTFASYGMNWVRQAPGKGLEWVSSISSSSSYIYYADSVKGRFTISRDNAKNSLYLQMNSLRAEDTAVYYCARDGGRTSYTATAHNWFDPWGQGTLVTVSS

SEQ ID NO: 104 - DNA, 378 bp, synthetic construct
  misc_feature 1..378: note = DNA Heavy Chain
  source 1..378: mol_type = other DNA
gaggtgcagctggtggagtctgggggaggcctggtcaagcctggggggtccctgagactctcctgtgcagcctctggattcaccttcgctagctatgggatgaactgggtccgccaggctccagggaaggggctggagtgggtctcatccattagtagttctagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctca

SEQ ID NO: 105 - protein, 12 aa, synthetic construct
  REGION 1..12: note = Light Chain CDR1
QSVLFSSNNKNY

SEQ ID NO: 106 - skipped

SEQ ID NO: 107 - protein, 9 aa, synthetic construct
  REGION 1..9: note = Light Chain CDR3
QQHASAPPT

SEQ ID NO: 108 - protein, 17 aa, synthetic construct
  REGION 1..17: note = Light Chain CDR1
KSSQSVLFSSNNKNYLA

SEQ ID NO: 109 - protein, 7 aa, synthetic construct
  REGION 1..7: note = Light Chain CDR2
WASTRES

SEQ ID NO: 110 - protein, 9 aa, synthetic construct
  REGION 1..9: note = Light Chain CDR3
QQHASAPPT

SEQ ID NO: 111 - protein, 113 aa, synthetic construct; identical to SEQ ID NO: 23
  REGION 1..113: note = Light Chain

SEQ ID NO: 112 - DNA, 339 bp, synthetic construct; identical to SEQ ID NO: 24
  misc_feature 1..339: note = DNA Light Chain
  source 1..339: mol_type = other DNA

SEQ ID NO: 113 - protein, 456 aa, synthetic construct
  REGION 1..456: note = Heavy Chain
EVQLVESGGGLVKPGGSLRLSCAASGFTFASYGMNWVRQAPGKGLEWVSSISSSSSYIYYADSVKGRFTISRDNAKNSLYLQMNSLRAEDTAVYYCARDGGRTSYTATAHNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - DNA, 1368 bp, synthetic construct
  misc_feature 1..1368: note = DNA Heavy Chain
  source 1..1368: mol_type = other DNA
gaggtgcagctggtggagtctgggggaggcctggtcaagcctggggggtccctgagactctcctgtgcagcctctggattcaccttcgctagctatgggatgaactgggtccgccaggctccagggaaggggctggagtgggtctcatccattagtagttctagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 115 - protein, 220 aa, synthetic construct; identical to SEQ ID NO: 27
  REGION 1..220: note = Light Chain

SEQ ID NO: 116 - DNA, 660 bp, synthetic construct; identical to SEQ ID NO: 28
  misc_feature 1..660: note = DNA Light Chain
  source 1..660: mol_type = other DNA

SEQ ID NO: 117 - protein, 452 aa, synthetic construct
  REGION 1..452: note = Heavy Chain
EVQLVESGGGLVKPGGSLRLSCAASGFTFASYGMNWVRQAPGKGLEWVSSISSSSSYIYYADSVKGRFTISRDNAKNSLYLQMNSLRAEDTAVYYCARDGGRTSYTATAHNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 118 - DNA, 1356 bp, synthetic construct
  misc_feature 1..1356: note = DNA Heavy Chain
  source 1..1356: mol_type = other DNA
gaggtgcagctggtggagtctgggggaggcctggtcaagcctggggggtccctgagactctcctgtgcagcctctggattcaccttcgctagctatgggatgaactgggtccgccaggctccagggaaggggctggagtgggtctcatccattagtagttctagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcttccaccaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgcccttcctgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggc

SEQ ID NO: 119 - protein, 8 aa, synthetic construct
  REGION 1..8: note = Heavy chain CDR1
GFTFRSYG

SEQ ID NO: 120 - protein, 8 aa, synthetic construct
  REGION 1..8: note = Heavy chain CDR2
ISSSGSYI

SEQ ID NO: 121 - protein, 19 aa, synthetic construct
  REGION 1..19: note = Heavy chain CDR3
ARDGGRTSYTATAHNWFDP

SEQ ID NO: 122 - protein, 9 aa, synthetic construct
  REGION 1..9: note = Heavy chain CDR1
FTFRSYGMN

SEQ ID NO: 123 - protein, 17 aa, synthetic construct
  REGION 1..17: note = Heavy chain CDR2
GISSSGSYIYYADSVKG

SEQ ID NO: 124 - protein, 19 aa, synthetic construct
  REGION 1..19: note = Heavy chain CDR3
ARDGGRTSYTATAHNWFDP

SEQ ID NO: 125 - protein, 126 aa, synthetic construct
  REGION 1..126: note = Heavy Chain
EVQLVESGGGLVKPGGSLRLSCAASGFTFRSYGMNWVRQAPGKGLEWVSGISSSGSYIYYADSVKGRFTISRDNAKNSLYLQMNSLRAEDTAVYYCARDGGRTSYTATAHNWFDPWGQGTLVTVSS

SEQ ID NO: 126 - DNA, 378 bp, synthetic construct
  misc_feature 1..378: note = DNA Heavy Chain
  source 1..378: mol_type = other DNA
gaggtgcagctggtggagtctgggggaggcctggtcaagcctggggggtccctgagactctcctgtgcagcctctggattcaccttccgtagctatgggatgaactgggtccgccaggctccagggaaggggctggagtgggtctcaggtattagtagtagtggtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctca

SEQ ID NO: 127 - protein, 12 aa, synthetic construct
  REGION 1..12: note = Light Chain CDR1
QSVLFSSNNKNY

SEQ ID NO: 128 - skipped

SEQ ID NO: 129 - protein, 9 aa, synthetic construct
  REGION 1..9: note = Light Chain CDR3
QQHASAPPT

SEQ ID NO: 130 - protein, 17 aa, synthetic construct
  REGION 1..17: note = Light Chain CDR1
KSSQSVLFSSNNKNYLA

SEQ ID NO: 131 - protein, 7 aa, synthetic construct
  REGION 1..7: note = Light Chain CDR2
WASTRES

SEQ ID NO: 132 - protein, 9 aa, synthetic construct
  REGION 1..9: note = Light Chain CDR3
QQHASAPPT

SEQ ID NO: 133 - protein, 113 aa, synthetic construct; identical to SEQ ID NO: 23
  REGION 1..113: note = Light Chain

SEQ ID NO: 134 - DNA, 339 bp, synthetic construct; identical to SEQ ID NO: 24
  misc_feature 1..339: note = DNA Light Chain
  source 1..339: mol_type = other DNA

SEQ ID NO: 135 - protein, 456 aa, synthetic construct
  REGION 1..456: note = Heavy Chain
EVQLVESGGGLVKPGGSLRLSCAASGFTFRSYGMNWVRQAPGKGLEWVSGISSSGSYIYYADSVKGRFTISRDNAKNSLYLQMNSLRAEDTAVYYCARDGGRTSYTATAHNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - DNA, 1368 bp, synthetic construct
  misc_feature 1..1368: note = DNA Heavy Chain
  source 1..1368: mol_type = other DNA
gaggtgcagctggtggagtctgggggaggcctggtcaagcctggggggtccctgagactctcctgtgcagcctctggattcaccttccgtagctatgggatgaactgggtccgccaggctccagggaaggggctggagtgggtctcaggtattagtagtagtggtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 137 - protein, 220 aa, synthetic construct; identical to SEQ ID NO: 27
  REGION 1..220: note = Light Chain

SEQ ID NO: 138 - DNA, 660 bp, synthetic construct; identical to SEQ ID NO: 28
  misc_feature 1..660: note = DNA Light Chain
  source 1..660: mol_type = other DNA

SEQ ID NO: 139 - protein, 452 aa, synthetic construct
  REGION 1..452: note = Heavy Chain
EVQLVESGGGLVKPGGSLRLSCAASGFTFRSYGMNWVRQAPGKGLEWVSGISSSGSYIYYADSVKGRFTISRDNAKNSLYLQMNSLRAEDTAVYYCARDGGRTSYTATAHNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 140 - DNA, 1356 bp, synthetic construct
  misc_feature 1..1356: note = DNA Heavy Chain
  source 1..1356: mol_type = other DNA
gaggtgcagctggtggagtctgggggaggcctggtcaagcctggggggtccctgagactctcctgtgcagcctctggattcaccttccgtagctatgggatgaactgggtccgccaggctccagggaaggggctggagtgggtctcaggtattagtagtagtggtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcttccaccaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgcccttcctgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggc

SEQ ID NO: 141 - protein, 8 aa, synthetic construct
  REGION 1..8: note = FLAG
DYKDDDDK

SEQ ID NO: 142 - protein, 6 aa, synthetic construct
  REGION 1..6: note = 6-HIS
HHHHHH

SEQ ID NO: 143 - protein, 9 aa, synthetic construct
  REGION 1..9: note = synthetic HA
YPYDVPDYA

SEQ ID NO: 144 - protein, 8 aa, synthetic construct
  REGION 1..8: note = consensus heavy chain CDR1
  VARIANT 5: note = Wherein Xaa can be Ser, Ala, or Arg
  VARIANT 6: note = Wherein Xaa can be Ser or Arg
  VARIANT 7: note = Wherein Xaa can be Thr or Tyr
  VARIANT 8: note = Wherein Xaa can be Gly or Ser
GFTFXXXX

SEQ ID NO: 145 - protein, 8 aa, synthetic construct
  REGION 1..8: note = consensus heavy chain CDR1
  VARIANT 5..8: note = Xaa can be any naturally occurring amino acid
GFTFXXXX

SEQ ID NO: 146 - protein, 8 aa, synthetic construct
  REGION 1..8: note = consensus heavy chain CDR2
  VARIANT 5: note = Wherein Xaa can be Ser or Gly
  VARIANT 6: note = Wherein Xaa can be Ser or Ala
ISSSXXYI

SEQ ID NO: 147 - protein, 8 aa, synthetic construct
  REGION 1..8: note = consensus heavy chain CDR2
  VARIANT 5..6: note = Xaa can be any naturally occurring amino acid
ISSSXXYI

SEQ ID NO: 148 - protein, 9 aa, synthetic construct
  REGION 1..9: note = consensus heavy chain CDR1
  VARIANT 4: note = Wherein Xaa can be Ser, Ala, or Arg
  VARIANT 5: note = Wherein Xaa can be Ser or Arg
  VARIANT 6: note = Wherein Xaa can be Thr or Tyr
  VARIANT 7: note = Wherein Xaa can be Gly or Ser
FTFXXXXMN

SEQ ID NO: 149 - protein, 17 aa, synthetic construct
  REGION 1..17: note = consensus heavy chain CDR2
  VARIANT 1: note = Wherein Xaa can be Gly or Ser
  VARIANT 6: note = Wherein Xaa can be Gly or Ser
  VARIANT 7: note = Wherein Xaa can be Ser or Ala
  VARIANT 10: note = Wherein Xaa can be Leu or Tyr
XISSSXXYIXYADSVKG

SEQ ID NO: 150 - protein, 9 aa, synthetic construct
  REGION 1..9: note = consensus heavy chain CDR1
  VARIANT 4..7: note = Xaa can be any naturally occurring amino acid
FTFXXXXMN

SEQ ID NO: 151 - protein, 17 aa, synthetic construct
  REGION 1..17: note = consensus heavy chain CDR2
  VARIANT 1: note = Xaa can be any naturally occurring amino acid
  VARIANT 6..7: note = Xaa can be any naturally occurring amino acid
  VARIANT 10: note = Xaa can be any naturally occurring amino acid
XISSSXXYIXYADSVKG

SEQ ID NO: 152-153 - skipped (2 entries)

SEQ ID NO: 154 - protein, 12 aa, synthetic construct
  REGION 1..12: note = Light chain CDR1
  VARIANT 3..5: note = Wherein Xaa can be any amino acid and up to 2 can be absent
  VARIANT 8..11: note = Wherein Xaa can be any amino acid and up to 4 can be absent
QSXXXSSXXXXY

SEQ ID NO: 155 - skipped

SEQ ID NO: 156 - protein, 9 aa, synthetic construct
  REGION 1..9: note = Light chain CDR3
  VARIANT 3..6: note = Xaa can be any naturally occurring amino acid
  VARIANT 8: note = Wherein Xaa can be any amino acid and can be absent
QQXXXXPXT

(19) United States
(12) Patent Application Publication
(10) Pub. No.: US 2026/0167716 A1
(43) Pub. Date: Jun. 18, 2026

(54) METHODS OF TREATING A TUMOR

(71) Applicant: SURFACE ONCOLOGY, LLC, Redwood City, CA (US)

(72) Inventors: Ricard MASIA, Cambridge, MA (US); Jonathan HILL, Boston, MA (US); Secil KOSEOGLU, Boston, MA (US); Benjamin H. LEE, Cambridge, MA (US); Yu YANG, Boston, MA (US)

(73) Assignee: SURFACE ONCOLOGY, LLC, Redwood City, CA (US)

(21) Appl. No.: 19/124,593

(22) PCT Filed: Oct. 30, 2023
(86) PCT No.: PCT/US2023/078163

§ 371 Date: Apr. 25, 2025

Related U.S. Application Data
(60) Provisional application No. 63/381,893, filed on Nov. 1, 2022.

(51) Int. Cl. C07K 16/28; A61K 39/395; A61K 45/06

(52) U.S. Cl. CPC C07K 16/2803; A61K 39/3955; A61K 45/06; C07K 2317/76; C07K 2317/92

(57) ABSTRACT

The present disclosure relates to methods of treating a tumor in a subject in need thereof comprising administering an IL-27 inhibiting agent, wherein the subject is identified as having a tumor that (i) expresses WSX-1, (ii) comprises one or more immune cell that expresses IL-27, (iii) or both (i) and (ii).

### CROSS REFERENCE TO RELATED APPLICATIONS

This application is a 35 U.S.C. § 371 National Stage of International Patent Application No. PCT/US2023/078163, filed Oct. 30, 2023, claiming benefit from U.S. Provisional Application No. 63/381,893, filed Nov. 1, 2022, the disclosures of which are incorporated herein in their entirety by reference, and priority is claimed to each of the foregoing.

### SEQUENCE LISTING

This application contains a Sequence Listing in computer readable form entitled “01219-0017-00PCT_ST26”, created Oct. 26, 2022, having a size of 240,443 Bytes, which is incorporated by reference herein.

### FIELD

The present disclosure relates generally to methods of treating a tumor in a subject in need thereof comprising administering compositions that modulate IL-27 signaling. More particularly, the present disclosure relates to methods of treating a tumor in a subject in need thereof comprising administering immunogenic compositions (e.g., antibodies, antibody fragments, and the like) that bind to IL-27 and modulate IL-27 signaling.

### BACKGROUND

In recent years, an increasing body of evidence suggests that the immune system operates as a significant barrier to tumor formation and progression. The principle that naturally occurring T cells with anti-tumor potential or activity exist in a patient with cancer has rationalized the development of immunotherapeutic approaches in oncology. Immune cells, such as T cells, macrophages, and natural killer cells, can exhibit anti-tumor activity and effectively control the occurrence and growth of malignant tumors. Tumor-specific or -associated antigens can induce immune cells to recognize and eliminate malignancies (Chen & Mellman, (2013) Immunity 39 (1): 1-10). In spite of the existence of tumor-specific immune responses, malignant tumors often evade or avoid immune attack through a variety of immunomodulatory mechanisms resulting in the failure to control tumor occurrence and progression (Motz & Coukos, (2013) Immunity 39 (1): 61-730). Indeed, an emerging hallmark of cancer is the exploitation of these immunomodulatory mechanisms and the disablement of anti-tumor immune responses, resulting in tumor evasion and escape from immunological killing (Hanahan and Weinberg (2011) Cell 144 (5): 646-674).

IL-27 is a heterodimeric cytokine, composed of two subunits (EBI3 and IL-27p28). IL-27 is structurally related to both the IL-12 and IL-6 cytokine families. IL-27 binds to and mediates signaling through a heterodimer receptor consisting of IL-27Rα (WSX1) and gp130 chains, which mediate signaling predominantly through STAT1 and STAT3. Initial reports characterized IL-27 as an immune-enhancing cytokine that supports CD4+ T cell proliferation, T helper (Th)1 cell differentiation, and IFN-γ production, often acting in concert with IL-12. Subsequent studies have shown that IL-27 displays complex immunomodulatory functions, resulting in either proinflammatory or anti-inflammatory effects depending on the biological context and experimental models being used. IL-27 may drive the expression of different immune-regulatory molecules in human cancer cells, which may support local derangement of the immune response in vivo (Fabbi et al., (2017) Mediators Inflamm 3958069. Published online 2017 Feb. 1. doi: 10.1155/2017/3958069, and references contained therein).

Despite the significant advances being made in cancer treatment and management, there is still an ongoing need for new and effective therapies for treating and managing cancer.

### SUMMARY OF THE DISCLOSURE

Some aspects of the present disclosure are directed to a method for treating a tumor in a subject comprising administering an IL-27 inhibiting agent to the subject, wherein the tumor is identified as being a WSX-1-positive tumor. Some aspects of the present disclosure are directed to a method for treating a tumor in a subject in need thereof, comprising: (i) identifying a subject having a WSX-1-positive tumor; and (ii) administering to the subject an IL-27 inhibiting agent. In some aspects, the WSX-1-positive tumor is identified by detecting WSX-1 expression in a tumor sample obtained from the subject.

Some aspects of the present disclosure are directed to a method for identifying a human subject afflicted with a tumor suitable for treatment with an IL-27 inhibiting agent, the method comprising detecting WSX-1 expression in a tumor sample obtained from the subject. In some aspects, the method further comprises administering an IL-27 inhibiting agent to the subject identified as having a WSX-1-positive tumor.

In some aspects, the tumor sample obtained from the subject is a tumor tissue biopsy. In some aspects, the tumor sample obtained from the subject is a formalin-fixed paraffin-embedded tumor sample. In some aspects, the tumor sample obtained from the subject comprises tumor cells, tumor infiltrating immune cells, or both.

In some aspects, at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, or at least about 50% of cells in the tumor sample express WSX-1. In some aspects, at least about 1% of cells in the tumor sample express WSX-1.

In some aspects, the WSX-1 expression is detected using an immunohistochemical (IHC) assay. In some aspects, the WSX-1 expression is detected using an automated IHC assay. In some aspects, the WSX-1 expression is scored using a tumor proportion score (TPS) and/or a combined positive score (CPS). In some aspects, the TPS or CPS is at least 10%, at least 20%, at least 30%, at least 50% or at least 60%. In some aspects, the WSX-1 expression is detected by contacting the tumor sample with an antibody or an antigen-binding portion thereof that specifically binds human WSX-1.

Some aspects of the present disclosure are directed to a method for treating a tumor in a subject comprising administering an IL-27 inhibiting agent to the subject, wherein one or more immune cells in a tumor sample obtained from the subject express IL-27.

Some aspects of the present disclosure are directed to a method for treating a tumor in a subject in need thereof, comprising: (i) identifying a subject having a tumor wherein one or more immune cells in a tumor sample obtained from the subject express IL-27; and (ii) administering to the subject an IL-27 inhibiting agent.

Some aspects of the present disclosure are directed to a method for identifying a human subject afflicted with a tumor suitable for treatment with an IL-27 inhibiting agent, the method comprising detecting IL-27 expression in a tumor sample obtained from the subject. In some aspects, the method further comprises administering an IL-27 inhibiting agent to the subject identified as having a tumor sample comprising one or more immune cells that express IL-27.

In some aspects, the tumor sample obtained from the subject is a tumor tissue biopsy. In some aspects, the tumor sample obtained from the subject is a formalin-fixed paraffin-embedded tumor sample. In some aspects, the tumor sample obtained from the subject comprises tumor cells, tumor infiltrating immune cells, or both. In some aspects, the one or more immune cells in the tumor sample comprise macrophages.

In some aspects, at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, or at least about 50% of the immune cells in the tumor sample express IL-27. In some aspects, at least about 1% of immune cells in the tumor sample express IL-27.

In some aspects, the IL-27 expression is detected using an immunohistochemical (IHC) assay. In some aspects, the IL-27 expression is detected using an automated IHC assay. In some aspects, the IL-27 expression is assessed using a tumor proportion score (TPS) and/or a combined positive score (CPS). In some aspects, the TPS or CPS is at least 10%, at least 20%, at least 30%, at least 50% or at least 60%. In some aspects, the IL-27 expression is detected by contacting the tumor sample with an antibody or an antigen-binding portion thereof that specifically binds human IL-27.

In some aspects, the IL-27 inhibiting agent reduces or blocks the interaction between IL-27 and WSX-1. In some aspects, the IL-27 inhibiting agent comprises a polypeptide or a small molecule. In some aspects, the IL-27 inhibiting agent comprises an antibody or an antigen-binding portion thereof that specifically binds to human IL-27 (“anti-IL-27 antibody”).

In some aspects, the IL-27 inhibiting agent increases expression of GBP5 and IRF1. In some aspects, the IL-27 inhibiting agent increases expression of GBP5 and IRF1 in NK cells and/or CD8+ T cells.

In some aspects, the anti-IL-27 antibody specifically binds to an epitope on human IL-27 comprising one or more amino acids of (i) amino acids 37 to 56 corresponding to SEQ ID NO: 2 (IL-27p28), (ii) amino acids 142 to 164 corresponding to SEQ ID NO: 2 (IL-27p28), or (iii) both (i) and (ii). In some aspects, the epitope comprises one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, or Glu164 of SEQ ID NO: 2 (IL-27p28). In some aspects, the epitope comprises Asp146, Arg149, and/or Phe153 of SEQ ID NO: 2 (IL-27p28). In some aspects, the epitope further comprises His150 and/or Leu156 of SEQ ID NO: 2 (IL-27p28). In some aspects, the epitope further comprises Gln37, Leu38, Glu42, Leu142, and/or Glu164 of SEQ ID NO: 2 (IL-27p28). In some aspects, the epitope further comprises Glu46, Val49, Ser50, and/or Leu162 of SEQ ID NO: 2 (IL-27p28). In some aspects, the epitope consists or consists essentially of Gln37, Leu38, Glu42, Glu46, Val49, Ser50, Leu142, Asp146, Arg149, His150, Phe153, Leu156, Leu162, and Glu164 of SEQ ID NO: 2 (IL-27p28). In some aspects, the epitope further comprises one or more amino acids of Leu53, Lys56, Asp143, Leu147, Arg152, Ala157, Gly159, Phe160, or Asn161 of SEQ ID NO: 2 (IL-27p28). In some aspects, the epitope further comprises one or more amino acids of Leu53, Lys56, Asp143, Arg145, Leu147, Arg152, Ala157, Gly159, Phe160, Asn161, or Pro163 of SEQ ID NO: 2 (IL-27p28). In some aspects, the epitope consists or consists essentially of Gln37, Leu38, Glu42, Glu46, Val49, Ser50, Leu53, Lys56, Leu142, Asp143, Asp146, Leu147, Arg149, His150, Arg152, Phe153, Leu156, Ala157, Gly159, Phe160, Asn161, Leu162, and Glu164 of SEQ ID NO: 2 (IL-27p28). In some aspects, the epitope consists or consists essentially of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164, of SEQ ID NO: 2 (IL-27p28).

In some aspects, IL-27 inhibitory agent comprises an antibody or an antigen-binding portion thereof that specifically binds human IL-27, wherein the antibody or the antigen binding portion thereof comprise heavy chain CDR1, heavy chain CDR2, heavy chain CDR3, light chain CDR1, light chain CDR2, and light chain CDR3, wherein (i) light chain CDR1 consists of N-XXXXXXLFSSNXKXYXX-C, light chain CDR3 consists of N-XXXASAXXX-C, heavy chain CDR2 consists of N-XXSSSXSYXYXXXXXXX-C, and heavy chain CDR3 consists of N-XXXXGRTSYTATXHNXXXX-C, wherein X is any amino acids.

In some aspects, IL-27 inhibitory agent comprises an antibody or an antigen-binding portion thereof that specifically binds human IL-27, wherein the antibody or the antigen binding portion thereof comprises a heavy chain CDR3 comprising the sequence set forth in SEQ ID NO: 121 or 124. In some aspects, IL-27 inhibitory agent comprises an antibody or an antigen-binding portion thereof that specifically binds human IL-27, wherein the antibody or the antigen binding portion thereof comprises a heavy chain CDR2 comprising the sequence set forth in SEQ ID NO: 120 or 123. In some aspects, IL-27 inhibitory agent comprises an antibody or an antigen-binding portion thereof that specifically binds human IL-27, wherein the antibody or the antigen binding portion thereof comprises a heavy chain CDR1 comprising the sequence set forth in SEQ ID NO: 119 or 122. In some aspects, the antibody or the antigen binding portion thereof comprises a light chain CDR3 comprising the sequence set forth in SEQ ID NO: 129 or 132. In some aspects, the antibody or the antigen binding portion thereof comprises a light chain CDR2 comprising the sequence set forth in SEQ ID NO: 128 or 131. In some aspects, the antibody or the antigen binding portion thereof comprises a light chain CDR1 comprising the sequence set forth in SEQ ID NO: 127 or 130.

In some aspects, the antibody or the antigen binding portion thereof comprises: (i) a heavy chain CDR1 comprising the amino acid sequence set forth in SEQ ID NO: 119, a heavy chain CDR2 comprising the amino acid sequence set forth in SEQ ID NO: 120, and a heavy chain CDR3 comprising the amino acid sequence set forth in SEQ ID NO: 121; or (i) a heavy chain CDR1 comprising the amino acid sequence set forth in SEQ ID NO: 122, a heavy chain CDR2 comprising the amino acid sequence set forth in SEQ ID NO: 123, and a heavy chain CDR3 comprising the amino acid sequence set forth in SEQ ID NO: 124.

In some aspects, the antibody or the antigen binding portion thereof comprises: (i) a light chain CDR1 comprising the amino acid sequence set forth in SEQ ID NO: 127, a light chain CDR2 comprising the amino acid sequence set forth in SEQ ID NO: 128, and a light chain CDR3 comprising the amino acid sequence set forth in SEQ ID NO: 129; or (ii) a light chain CDR1 comprising the amino acid sequence set forth in SEQ ID NO: 130, a light chain CDR2 comprising the amino acid sequence set forth in SEQ ID NO: 131, and a light chain CDR3 comprising the amino acid sequence set forth in SEQ ID NO: 132.

In some aspects, the antibody or the antigen binding portion thereof comprises: a heavy chain CDR1 comprising the amino acid sequence set forth in SEQ ID NO: 119, a heavy chain CDR2 comprising the amino acid sequence set forth in SEQ ID NO: 120, a heavy chain CDR3 comprising the amino acid sequence set forth in SEQ ID NO: 121, a light chain CDR1 comprising the amino acid sequence set forth in SEQ ID NO: 127, a light chain CDR2 comprising the amino acid sequence set forth in SEQ ID NO: 128, and a light chain CDR3 comprising the amino acid sequence set forth in SEQ ID NO: 129.

In some aspects, the antibody or the antigen binding portion thereof comprises: a heavy chain CDR1 comprising the amino acid sequence set forth in SEQ ID NO: 122, a heavy chain CDR2 comprising the amino acid sequence set forth in SEQ ID NO: 123, a heavy chain CDR3 comprising the amino acid sequence set forth in SEQ ID NO: 124 a light chain CDR1 comprising the amino acid sequence set forth in SEQ ID NO: 130, a light chain CDR2 comprising the amino acid sequence set forth in SEQ ID NO: 131, and a light chain CDR3 comprising the amino acid sequence set forth in SEQ ID NO: 132.

In some aspects, the antibody or the antigen binding portion thereof comprises a heavy chain variable region comprising an amino acid sequence that has at least about 85%, at least about 90%, at least about 95%, at least about 96%, at least about 97%, at least about 98%, or at least about 99% sequence identity to the amino acid sequence set forth in SEQ ID NO: 125. In some aspects, the antibody or the antigen binding portion thereof comprises a heavy chain variable region comprising the amino acid sequence set forth in SEQ ID NO: 125. In some aspects, the antibody or the antigen binding portion thereof comprises a light chain variable region comprising an amino acid sequence that has at least about 85%, at least about 90%, at least about 95%, at least about 96%, at least about 97%, at least about 98%, or at least about 99% sequence identity to the amino acid sequence set forth in SEQ ID NO: 133. In some aspects, the antibody or the antigen binding portion thereof comprises a light chain variable region comprising the amino acid sequence set forth in SEQ ID NO: 133. In some aspects, the antibody or the antigen binding portion thereof comprises a heavy chain variable region comprising the amino acid sequence set forth in SEQ ID NO: 125 and a light chain variable region comprising the amino acid sequence set forth in SEQ ID NO: 133. In some aspects, the antibody or the antigen binding portion thereof comprises a heavy chain comprising the amino acid sequence set forth in SEQ ID NO: 135. In some aspects, the antibody or the antigen binding portion thereof comprises a heavy chain comprising the amino acid sequence set forth in SEQ ID NO: 139. In some aspects, the antibody or the antigen binding portion thereof comprises a light chain comprising the amino acid sequence set forth in SEQ ID NO: 137. In some aspects, the antibody or the antigen binding portion thereof comprises a heavy chain comprising the amino acid sequence set forth in SEQ ID NO: 135 and a light chain comprising the amino acid sequence set forth in SEQ ID NO: 137. In some aspects, the antibody or the antigen binding portion thereof comprises a heavy chain comprising the amino acid sequence set forth in SEQ ID NO: 139 and a light chain comprising the amino acid sequence set forth in SEQ ID NO: 137.

In some aspects, the cancer is selected from lung cancer (e.g., non-small cell lung cancer), sarcoma, testicular cancer, ovarian cancer, pancreas cancer, breast cancer (e.g., triple-negative breast cancer), melanoma, head and neck cancer (e.g., squamous head and neck cancer), colorectal cancer, bladder cancer, endometrial cancer, prostate cancer, thyroid cancer, hepatocellular carcinoma (HCC), gastric cancer, brain cancer, lymphoma (e.g., DL-BCL), leukemia (e.g., AML), renal cancer (e.g., renal cell carcinoma (RCC), e.g., clear cell RCC and/or non-clear cell RCC), and any combination thereof.

In some aspects, the method further comprises administering an additional therapeutic agent to the subject. In some aspects, the additional therapeutic agent is administered before the antibody or antigen-binding portion thereof, after the antibody or antigen-binding portion thereof, or concurrently with the antibody or antigen-binding portion thereof. In some aspects, the additional therapeutic agent comprises a chemotherapy, a targeted anti-cancer therapy, an oncolytic drug, a cytotoxic agent, an immune-based therapy, a cytokine, surgical procedure, a radiation procedure, an activator of a costimulatory molecule, an inhibitor of an inhibitory molecule, a vaccine, a cellular immunotherapy, a biologic agent, or a combination thereof.

In some aspects, the additional therapeutic agent comprises a PD-1 antagonist, a PD-L1 inhibitor, a TIM-3 inhibitor, a LAG-3 inhibitor, a TIGIT inhibitor, a CD112R inhibitor, a TAM inhibitor, a STING agonist, a 4-1BB agonist, a multityrosine kinase inhibitor (e.g., a VEGFR inhibitor), an anti-VEGF blocking antibody, a CTLA-4 antagonist, a HIF2 antagonist, a TGFb antagonist, an mTOR inhibitor, an adenosine pathway inhibitor (e.g., an anti-CD73 antibody, an anti-CD39 antibody, an anti-A2AR antibody, an anti-A2BR, or any combination thereof), an anti-CCR8 antibody, a cytokine-based regimen (e.g., IL-2 or IFN-a), a PARP inhibitor, or a combination thereof.

In some aspects, the additional therapeutic agent comprises a PD-1 antagonist. In some aspects, the PD-1 antagonist is selected from the group consisting of: PDR001, nivolumab, pembrolizumab, pidilizumab, MEDI0680, REGN2810, TSR-042, PF-06801591, and AMP-224.

In some aspects, the PD-L1 inhibitor is selected from the group consisting of: FAZ053, Atezolizumab, Avelumab, Durvalumab, and BMS-936559.

In some aspects, the additional therapeutic agent is selected from the group consisting of Sunitinib (SUTENT®), Cabozantinib (CABOMETYX®), Axitinib (INLYTA®), Lenvatinib (LENVIMA®), Everolimus (AFINITOR®), Bevacizumab (AVASTIN®), epacadostat, NKTR-214 (CD-122-biased agonist), Tivozanib (FOTIVDA®), abexinostat, Ipilimumab (YERVOY®), tremelimumab, Pazopanib (VOTRIENT®), Sorafenib (NEXAVAR®), Temsirolimus (TORISEL®), Ramucirumab (CYRAMZA®), niraparib, savolitinib, vorolanib (X-82), Regorafenib (STIVARGO®), Donafenib (multikinase inhibitor), Camrelizumab (SHR-1210), pexastimogene devacirepvec (JX-594), Ramucirumab (CYRAMZA®), apatinib (YN968D1), encapsulated doxorubicin (THERMODOX®), Tivantinib (ARQ197), ADI-PEG 20, binimetinib, apatinib mesylate, nintedanib, lirilumab, Nivolumab (OPDIVO®), Pembrolizumab (KEYTRUDA®), Atezolizumab (TECENTRIQ®), Avelumab (BAVENCIO®), Durvalumab (IMFIMZI®), Cemiplimab-rwlc (LIBTAYO®), tislelizumab, and spartalizumab.

In some aspects, the additional therapeutic agent is a TIM-3 inhibitor. In some aspects, the TIM-3 inhibitor is MGB453 or TSR-022.

In some aspects, the additional therapeutic agent is a LAG-3 inhibitor. In some aspects, the LAG-3 inhibitor is selected from the group consisting of LAG525, BMS-986016, and TSR-033.

In some aspects, the additional therapeutic agent is a TIGIT inhibitor. In some aspects, the additional therapeutic agent is a CD112R inhibitor. In some aspects, the additional therapeutic agent is a TAM (Axl, Mer, Tyro) inhibitor. In some aspects, the additional therapeutic agent is a 4-1BB agonist. In some aspects, the additional therapeutic agent is a Tyrosine Kinase Inhibitor (TKI).

Some aspects of the present disclosure are directed to a kit comprising: (i) an antibody or antigen-binding portion thereof that specifically binds human WSX-1; (ii) an IL-27 inhibiting agent; and (iii) instructions for use of (i) and (ii) in a method disclosed herein.

Some aspects of the present disclosure are directed to a kit comprising: (i) an antibody or antigen-binding portion thereof that specifically binds human IL-27; (ii) an IL-27 inhibiting agent; and (iii) instructions for use of (i) and (ii) in a method disclosed herein.

### BRIEF DESCRIPTION OF THE DRAWINGS

FIGS. 1A-1J present data showing WSX-1 mRNA expression (FIG. 1A) and protein expression (FIGS. 1B-1J). FIG. 1A is a box plot illustrating the expression of WSX-1 mRNA in non-heme cell lines. FIGS. 1B-1F are images of IHC for WSX-1 protein in FFPE tumor samples for lung adenocarcinoma (FIG. 1B), lung squamous cell carcinoma (SCC), ovarian cancer, head and neck squamous cell carcinoma (HNSCC), and triple negative breast cancer (TNBC). FIG. 1G is a bar graph summarizing the pertages of cases that exhibit tumor cell staining for WSX-1 by IHC. FIGS. 1H-1I are example images of lung adenocarcinoma that exhibits tumor cell staining for WSX-1 in both the primary tumor (FIG. 1H) and in a synchronous lymph node metastasis (FIG. 1I). FIG. 1J is a bar graph illustrating the percentage of NSCLC cases metastatic to the lymph node that exhibit tumor cell staining for WSX-1 by IHC, grouped by the WSX-1 status of the primary tumor.

FIGS. 2A-2F are examples of anti-IL-27 immunohistochemistry images of tumor samples from lung SCC (FIG. 2A), clear cell renal cell carcinoma (ccRCC; FIG. 2B), gastic cancer (FIG. 2C), hepatocellular cancer (HCC; FIG. 2D), HNSCC (FIG. 2E), and lung adenocarcinoma (FIG. 2F), showing positive cells in the tumor microenvironment (TME) that are morphologically consistent with tumor-associated macrophages (TAMs). FIG. 2G is a graphical represenations of the density of IL-27+ cells (cells/mm²) across various cancer types. FIG. 2H is an sc-RNA seq analysis of immune atlas for different cancer tumor cells for the overall IL-27 expression in all cancer types of the study.

FIGS. 3A-3C are graphical representations of the correlation between IL-27 expression and PD-L1 expression in NSCLC (FIG. 3A), gastric cancer (FIG. 3B), and HCC (FIG. 3C). **p<0.01; ***p<0.001. FIGS. 3D-3I are examples of IHC for PD-L1 and IL-27 in gastric cancer samples with a range of CPS scores. CPS score correlates with the density of IL-27+ cells (left and center), although some tumor samples with CPS<1 (negative for PD-L1) contain IL-27+ cells (right). All images at 17× magnification.

FIGS. 4A-4I are example images of IHC for IL-27 (FIGS. 4A, 4D, and 4G), WSX-1 (FIGS. 4B, 4E, and 4H), and PD-L1 (FIGS. 4C, 4F, and 4I) performed on serial sections of a normal human tonsil (FIGS. 4A-4C) and lung SCC (FIGS. 4D-4I).

FIG. 5 shows example images of IHC for IL-27 performed on a NSCLC tumor sample. Center image: 0.9× magnification; insets: 12× magnification.

FIGS. 6A-6D are example images of IHC for WSX-1 performed on NSCLC tumor samples. Cell staining for WSX-1 is shown in the peritumoral immune cell infiltrate (FIG. 6A, 0.55× magnification), Tertiary Lymphoid Structures (TLS; FIG. 6B, 3.5× magnification), Follicular Dendritic Cells (FDCs) within the germinal centers of TLS (FIG. 6C, 12× magnification), and outside TLS (FIG. 6D, 20× magnification).

FIG. 7A provides example images of IHC for IL-27 (left) and WSX-1 (right) performed on NSCLC tumor samples obtained as whole sections from individual lobectomy specimens. FIG. 7B provides a graph of the semi-quantitative scoring of immune cells in tumor tissue area. Bars show mean±SEM for n=44 NSCLC lobectomy specimens.

FIGS. 8A-8B provides example images of IHC for IL-27 (left) and WSX-1 (right) on FFPE samples of NSCLC draining lymph nodes obtained from individual lobectomy specimens in lymph node metastases (FIG. 8A; 10× magnification) and draning lymph nodes that do not contain tumors (FIG. 8B; 7× magnification). FIG. 8C provides example images of IHC for IL-27 (left) and WSX-1 (right) on control lymph nodes.

FIGS. 9A-9B provide example images of IHC for IL-27 (FIG. 9A; 20× magnification) and WSX-1 (FIG. 9B; 20× magnification) in a subset of NSCLC samples. FIG. 9C provides a graph of the percent of cases with positive staining in tumor cells. Bars show mean±SEM for n=44 NSCLC lobectomy specimens.

FIG. 10A provides example images of IHC for IL-27 (left) and WSX-1 (center) and CD8 (right) for in NSCLC cases (2.2× magnification). FIG. 10B provides example images of IHC for IL-27 (left) and PD-L1 (center) and CD8 (right) for in NSCLC cases (4× magnification [top] and 10× magnification [bottom]).

FIGS. 11A-11B provide example images of IHC for IL-27 and PD-L1 for in NSCLC cases. FIG. 11A shows IHC in lung adenocarcinoma (4× magnification [top] and 20× magnification [bottom]). FIG. 11B shows IHC in lung squamaous cell carcinoma (6× magnification [top] and 18× magnification [bottom]).

FIGS. 12A-12D are graphical representations of the correlation between IL-27 and WSX-1 with expression of PD-L1 in tumors classified using the Tumor Proportional Score (TPS) system (FIGS. 12A, 12C) or Combined Proportional Score (CPS) system (FIGS. 12B, 12C).

FIGS. 13A-13B are graphical representations of the correlation between IL-27 and WSX-1 in patients who subsequently respond to immune checkpoint blockade (ICP) (FIG. 13A) and between response to ICP and PD-L1 status (FIG. 13B). Data points are mean±SEM for n=24 NSCLC lobectomy specimens. FIGS. 13C-13D are graphical representations of the correlation between immune cell expression of IL-27 and WSX-1 in patients a who received ICP as first-line therapy (FIG. 13C) and those who received ICP as second-line or higher therapy (FIG. 13D). TPS: Tumor Proportional Score, CPS: Combined Proportional Score. *: p<0.01 by Student's T-test. All other comparisons are not statistically significant. PD: Progressive Disease, SD: Stable Disease, PR: Partial Response, CR: Complete Response. Number of patients in each response category shown below response label.

FIGS. 14A-14B are graphical representations of IHC scoring on archival specimens from NSCLC patients subsequently treated with SRF388 using TAM scoring (FIG. 14A) or positive IL-27+ immune cells as a percentage of tumor area (FIG. 14B). Squares indicate archival biopsy specimens; circles indicate archival resection specimens. Black circles/squares indicate partial response to SRF388 monotherapy; gray circles/squares indicate stable disease; white circles/squares indicate progressive disease. FIGS. 14C-14F provide IHC images for IL-27 on archival lung resection specimen (FIG. 14C, 20× magnification), including adjacent to Tertiary Lymphoid Structures (TLS) (FIG. 14D, 15× magnification, asterisk indicates TLS) and FIG. 14F residual lymph node tissue (FIG. 14E, 8× magnification, and 20× magnification).

FIG. 15A is a graphical representation of IHC scoring on archival specimens from from hepatocellular carcinoma (HCC) patients subsequently treated with SRF388 in combination with atezolizumab and bevacizumab using TAM scoring FIGS. 15B-15F provide example images of IHC for IL-27 on patients who responded to SRF388 combination therapy (FIGS. 15B, 15E and 15F, 20× magnification). FIG. 15C and FIG. 15D focus on IL-27+ macrophages of FIG. 15B.

FIG. 16A provides an experimental outline for the single cell RNA-sequencing experiment comparing IL-27 to different interferons. Seurat-based clustering was used to identify cell subsets that were assigned based on differential gene expression. FIG. 16B provides UMAP representations of the aggregated data from all conditions identifying different cell populations.

FIG. 17A provides a schematic overview of IL-27 and Type 2/IFNG and Type 1/IFNB1 interferon transcript expression in activated PBMC based on scRNA-seq. FIG. 17B provides a heatmap projection of IL-27, IFNG, and IFNB1 transcript expression by different cell types in activated PBMC. FIG. 17C provides a point plot of IL-27 expression in activated PBMC after stimulation with different cytokines. FIGS. 18A-C provide data showing IL-27 and interferons upregulate the expression of several canonical interferon stimulated genes. FIG. 18A provides volcano plots showing genes upregulated or down-regulated in PBMC by the indicated cytokine.

FIG. 18B provides a Venn diagram showing the overlap in the top 100 genes upregulated by IFNB1, IFNG, and IL-27. FIG. 18C provides a heatmap representation of the 17 common genes from the top 100 genes upregulated by IFNB1, IFNG, and IL-27.

FIG. 19A provides a heatmap representation of immune checkpoint receptor gene expression showing similar upregulation of immunoregulatory receptors TIM-3 (HAVCR2), PD-L1 (CD274), TIGIT, and LAG3 by IL-27 or Type 1 or Type 2 IFNs in CD4+ T cells, CD8+ T cells, and CD14 hi monocytes. FIG. 19B provides a heatmap representation of cytokine gene expression showing different alterations in transcripts for GM-CSF (CSF2), IFNG, IL17A, IL17F, and IL10 by IL-27 or Type 1 or Type 2 interferons in CD4+ T cells and proliferating T cells (defined by MK167+ or having a Seurat assignment of G2M or S phase, but similar IFNG upregulation in CD8+ T cells by all 3 cytokines).

FIG. 20A provides UMAP representation of cells from activated PBMC that were cultured with different cytokines (Ctrl, IFNB1, IFNG, IL-27). Darker coloration represents high expression of the composite IFN gene signature, while lighter coloration is low expression. FIG. 20B provides a heatmap representation of cytokine receptor expression across different immune cell populations. Note the low expression of Type 2 interferon receptors (IFNR) on CD4+/CD8+ T cells and NK cells.

FIG. 21A provides flow cytometry data measuring STAT1 phosphorylation was measured in human PBMC after incubation for 30 min with the indicated cytokines. Quantitative statistical analysis of cytokine induced pSTAT1 (geometric mean fluorescent intensity, gMFI) in different cell types from PBMC across several healthy donors (n=12). FIG. 21B provides graphical data showing type 1 and IL-27 robustly induce STAT1 phosphorylation in T cells, NK cells and monocytes, while Type 2 (IFNG)-mediated STAT1 phosphorylation primarily occurs in monocytes (representative data from several experiments shown).

FIG. 22A provides cytokine gene expression signatures displayed as a composite signature (dark=high, light=low), highlighting the CD8 and NK cell populations that were used for the volcano plot analysis. FIG. 22B provides volcano plot analysis of differential gene expression by IL-27 compared to IFNB1 or IFNG in NK and CD8 T cells. FIG. 22C is a heatmap representation of genes preferentially regulated by IL-27, Type 1 or Type 2 IFNs in different immune cell populations. Note T cells include both CD8+ and CD4+ T cells.

FIG. 23A provides a violin plot of GBP5 transcript expression in T cells and NK cells after stimulation with IL-27 or Type 1 or Type 2 IFNs. FIG. 23B provides flow cytometry data of intracellular GBP5 expression in different cell types after stimulating PBMC with the indicated cytokines for 24 hr (representative data from several experiments shown). FIG. 23C provides example immunohistochemistry (IHC) images from treatment naïve NSCLC patient samples showing IL-27+ macrophages colocalized with GBP5+ T-cell-rich areas in the TME along with PD-L1+ immune cells. T-cell-rich areas are highlighted by CD4 IHC.

### DETAILED DESCRIPTION

Some aspects of the present disclosure are directed to methods for treating a tumor in a subject comprising administering an IL-27 inhibiting agent to the subject, wherein the tumor is identified (i) as being a WSX-1-positive tumor or (ii) as comprising one or more immune cells within the tumor that express IL-27. Some aspects of the present disclosure are directed to a method for treating a tumor in a subject in need thereof, comprising: (i) identifying a subject having (a) a WSX-1-positive tumor or (b) a tumor comprising one or more immune cells within the tumor that express IL-27; and (ii) administering to the subject an IL-27 inhibiting agent. Some aspects of the present disclosure are directed to a method for identifying a human subject afflicted with a tumor suitable for treatment with an IL-27 inhibiting agent, the method comprising detecting (i) WSX-1 expression or (ii) IL-27 expression in a tumor sample obtained from the subject.

### I. Definitions

Terms used in the claims and specification are defined as set forth below unless otherwise specified.

It must be noted that, as used in the specification and the appended claims, the singular forms “a,” “an” and “the” include plural references unless the context clearly dictates otherwise.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

As used herein, the term “agonist” refers to any molecule that partially or fully promotes, induces, increases, and/or activates a biological activity of a native polypeptide disclosed herein. Suitable agonist molecules specifically include agonist antibodies or antibody fragments, fragments or amino acid sequence variants of native polypeptides, peptides or proteins. In some aspects, activation in the presence of the agonist is observed in a dose-dependent manner. In some aspects, the measured signal (e.g., biological activity) is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% higher than the signal measured with a negative control under comparable conditions. Also disclosed herein, are methods of identifying agonists suitable for use in the methods of the disclosure. For example, these methods include, but are not limited to, binding assays such as enzyme-linked immuno-absorbent assay (ELISA), FORTE BIO® systems, and radioimmunoassay (RIA). These assays determine the ability of an agonist to bind the polypeptide of interest (e.g., a receptor or ligand) and therefore indicate the ability of the agonist to promote, increase or activate the activity of the polypeptide. Efficacy of an agonist can also be determined using functional assays, such as the ability of an agonist to activate or promote the function of the polypeptide. For example, a functional assay may comprise contacting a polypeptide with a candidate agonist molecule and measuring a detectable change in one or more biological activities normally associated with the polypeptide. The potency of an agonist is usually defined by its EC₅₀ value (concentration required to activate 50% of the agonist response). The lower the EC₅₀ value the greater the potency of the agonist and the lower the concentration that is required to activate the maximum biological response.

The term “ameliorating” refers to any therapeutically beneficial result in the treatment of a disease state, e.g., cancer, including prophylaxis, lessening in the severity or progression, remission, or cure thereof.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

As used herein, an “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, larger “peptide insertions,” can also be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

When referring to a protein, mRNA or a marker, such as those described herein, the terms “level of expression” or “expression level” in general are used interchangeably and generally refer to a detectable amount of a protein, mRNA, or marker in a biological sample. In some aspects, a detectable amount or detectable level of a protein, mRNA or a marker is associated with a likelihood of a response to an agent, such as those described herein. “Expression” generally refers to the process by which information contained within a gene is converted into the structures (e.g., a protein marker, such as WSX-1 or IL-27) present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs). “Elevated expression,” “elevated expression levels,” or “elevated levels” refers to an increased expression or increased levels of a substance within a sample relative to a control sample, such as an individual or individuals who are not suffering from the disease or disorder (e.g., cancer) or an internal control. In some aspects, the elevated expression of a substance (e.g., a protein marker, such as WSX-1 or IL-27) in a sample refers to an increase in the amount of the substance of about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% relative to the amount of the substance in a control sample, as determined by techniques known in the art (e.g., FACS). “Reduced expression,” “reduced expression levels,” or “reduced levels” refers to a decrease expression or decreased levels of a substance (e.g., a protein marker) in an individual relative to a control, such as an individual or individuals who are not suffering from the disease or disorder (e.g., cancer) or an internal control. In some aspects, reduced expression is little or no expression. In some aspects, the reduced expression of a substance (e.g., a protein marker) in a sample refers to a decrease in the amount of the substance of about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% relative to the amount of the substance in a control sample, as determined by techniques known in the art (e.g, FACS).

As used herein, the term “angiogenesis” or “neovascularization” refers to the process by which new blood vessels develop from pre-existing vessels (Varner et al., (1999) Angiogen. 3:53-60; Mousa et al., (2000) Angiogen. Stim. Inhib. 35:42-44; Kim et al., (2000) Amer. J. Path. 156:1345-1362; Kim et al., (2000) J. Biol. Chem. 275:33920-33928; Kumar et al. (2000) Angiogenesis: From Molecular to Integrative Pharm. 169-180). Endothelial cells from pre-existing blood vessels or from circulating endothelial stem cells (Takahashi et al., (1995) Nat. Med. 5:434-438; Isner et al., (1999) J. Clin. Invest. 103:1231-1236) become activated to migrate, proliferate, and differentiate into structures with lumens, forming new blood vessels, in response to growth factor or hormonal cues, or hypoxic or ischemic conditions. During ischemia, such as occurs in cancer, the need to increase oxygenation and delivery of nutrients apparently induces the secretion of angiogenic factors by the affected tissue; these factors stimulate new blood vessel formation. Several additional terms are related to angiogenesis.

The terms “inhibitor,” “inhibiting agent,” and “antagonist,” as used herein, can be used interchangeably to refer to any molecule that partially or fully blocks, reduces, inhibits, or neutralizes a biological activity of a native polypeptide disclosed herein. Suitable inhibiting agents specifically include antagonist antibodies or antibody fragments, fragments or amino acid sequence variants of native polypeptides, peptides or proteins. In some aspects, inhibition in the presence of the inhibiting agent is observed in a dose-dependent manner. In some aspects, the measured signal (e.g., biological activity) is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% lower than the signal measured with a negative control under comparable conditions. Also disclosed herein, are methods of identifying inhibiting agents suitable for use in the methods of the disclosure. For example, these methods include, but are not limited to, binding assays such as enzyme-linked immuno-absorbent assay (ELISA), ForteBio® systems, radioimmunoassay (RIA), Meso Scale Discovery assay (e.g., Meso Scale Discovery Electrochemiluminescence (MSD-ECL), and bead-based Luminex® assay. These assays determine the ability of an inhibiting agent to bind the polypeptide of interest (e.g., a receptor or ligand) and therefore indicate the ability of the agent to inhibit, neutralize or block the activity of the polypeptide. Efficacy of an inhibiting agent can also be determined using functional assays, such as the ability of an agent to inhibit the function of the polypeptide or an agonist. For example, a functional assay may comprise contacting a polypeptide with a candidate inhibiting agent molecule and measuring a detectable change in one or more biological activities normally associated with the polypeptide. The potency of an inhibiting agent is usually defined by its IC₅₀ value (concentration required to inhibit 50% of the agonist response). The lower the IC₅₀ value the greater the potency of the inhibiting agent and the lower the concentration that is required to inhibit the maximum biological response.

As used herein, the phrase “antibody that antagonizes human IL-27, or an antigen binding portion thereof” refers to an antibody that antagonizes at least one art-recognized activity of human IL-27 (e.g., IL-27 biological activity and/or downstream pathway(s) mediated by IL-27 signaling or other IL-27-mediated function), for example, relating to a decrease (or reduction) in human IL-27 activity that is at least 5%, 10%, 20%, 30%, 40%, 50%, 60%, 70%, 80%, 90%, or more. Additional examples of IL-27 biological activities and/or downstream pathway(s) mediated by IL-27 signaling or other IL-27-mediated function are described in additional detail below and elsewhere herein.

As used herein, the term “anti-IL-27 antagonist antibody” (interchangeably termed “anti-IL-27 antibody”) refers to an antibody that specifically binds to IL-27 and inhibits IL-27 biological activity and/or downstream pathway(s) mediated by IL-27 signaling or other IL-27-mediated function. An anti-IL-27 antagonist antibody encompasses antibodies that block, antagonize, suppress, inhibit or reduce an IL-27 biological activity (e.g., ligand binding, enzymatic activity), including downstream pathways mediated by IL-27 signaling or function, such as receptor binding and/or elicitation of a cellular response to IL-27 or its metabolites. In some aspects, an anti-IL-27 antagonist antibody provided by the disclosure binds to human IL-27 and prevents, blocks, or inhibits binding of human IL-27 to its cognate or normal receptor (e.g., IL-27 receptor), or one or more receptor subunits (e.g., gp130 and/or IL-27Rα (also known as WSX1/TCCR)). In some aspects, the anti-IL-27 antagonist antibody prevents, blocks, or inhibits the binding of human IL-27 to the gp130. In some aspects, the anti-IL-27 antagonist antibody prevents, blocks, or inhibits the binding of human IL-27 to the IL-27Ra. In some aspects, the anti-IL-27 antagonist antibody prevents, blocks, or inhibits the dimerization of IL-27 monomers. In some aspects, the anti-IL-27 antibody does not specifically bind to the EBI3 monomer. In some aspects, the anti-IL-27 antibody specifically binds to the IL-27p28 monomer. In some aspects, the anti-IL-27 antibody specifically binds to a non-contiguous epitope comprising P28, but does not bind to the EBI3 monomer. In some aspects, the anti-IL-27 antibody inhibits or reduces STAT1 and/or STAT3 phosphorylation in a cell. In some aspects, the anti-IL-27 antibody inhibits or reduces inhibition of CD161 expression in a cell (e.g., ameliorates or relieves IL-27 mediated inhibition of CD161 expression in a cell). In some aspects, the anti-IL-27 antibody inhibits or reduces PD-L1 expression in a cell. In some aspects, the anti-IL-27 induces or enhances PD-1-mediated secretion of one or more cytokines from a cell. In some aspects, the anti-IL-27 antibody alters the expression of TIM-3 in a cell. In some aspects, an anti-IL-27 antagonist antibody binds to human IL-27 and stimulates or enhances an anti-tumor response. In some aspects, the anti-IL-27 antagonist antibody binds to human IL-27 with an affinity of 15 nM or less. In some aspects, the anti-IL-27 antagonist antibody binds to human IL-27 and comprises a wild type or mutant IgG1 heavy chain constant region or a wild type or mutant IgG4 heavy chain constant region. Examples of anti-IL-27 antagonist antibodies are provided herein.

As used herein, the term “antibody” refers to a whole antibody comprising two light chain polypeptides and two heavy chain polypeptides. Whole antibodies include different antibody isotypes including IgM, IgG, IgA, IgD, and IgE antibodies. The term “antibody” includes a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, dogs, cats, rabbits, guinea pigs, gerbils, hamsters, rats, and mice. The antibody can be a purified or a recombinant antibody. As used herein, the term “antibody fragment,” “antigen-binding fragment,” or similar terms refer to a fragment of an antibody that retains the ability to bind to a target antigen (e.g., IL-27) and inhibit the activity of the target antigen. Such fragments include, e.g., a single chain antibody, a single chain Fv fragment (scFv), an Fd fragment, an Fab fragment, an Fab′ fragment, or an F(ab′) 2 fragment. An scFv fragment is a single polypeptide chain that includes both the heavy and light chain variable regions of the antibody from which the scFv is derived. In addition, intrabodies, minibodies, triabodies, and diabodies are also included in the definition of antibody and are compatible for use in the methods described herein. See, e.g., Todorovska et al., (2001) J. Immunol. Methods 248 (1): 47-66; Hudson and Kortt, (1999) J. Immunol. Methods 231 (1): 177-189; Poljak, (1994) Structure 2 (12): 1121-1123; Rondon and Marasco, (1997) Annu. Rev. Microbiol. 51:257-283, the disclosures of each of which are incorporated herein by reference in their entirety.

As used herein, the term “antibody fragment” also includes, e.g., single domain antibodies such as camelized single domain antibodies. See, e.g., Muyldermans et al., (2001) Trends Biochem. Sci. 26:230-235; Nuttall et al., (2000) Curr. Pharm. Biotech. 1:253-263; Reichmann et al., (1999) J. Immunol. Meth. 231:25-38; PCT application publication nos. WO 94/04678 and WO 94/25591; and U.S. Pat. No. 6,005,079, all of which are incorporated herein by reference in their entireties. In some aspects, the disclosure provides single domain antibodies comprising two VH domains with modifications such that single domain antibodies are formed.

In some aspects, an antigen-binding fragment includes the variable region of a heavy chain polypeptide and the variable region of a light chain polypeptide. In some aspects, an antigen-binding fragment described herein comprises the CDRs of the light chain and heavy chain polypeptide of an antibody.

The term “antigen presenting cell” or “APC” is a cell that displays foreign antigen complexed with MHC on its surface. T cells recognize this complex using T cell receptor (TCR). Examples of APCs include, but are not limited to, B cells, dendritic cells (DCs), peripheral blood mononuclear cells (PBMC), monocytes (such as THP-1), B lymphoblastoid cells (such as C1R.A2, 1518 B-LCL) and monocyte-derived dendritic cells (DCs). Some APCs internalize antigens either by phagocytosis or by receptor-mediated endocytosis.

The term “antigen presentation” refers to the process by which APCs capture antigens and enables their recognition by T cells, e.g., as a component of an MHC-I and/or MHC-II conjugate.

As used herein, the term “apoptosis” refers to the process of programmed cell death that occurs in multicellular organisms (e.g. humans). The highly regulated biochemical and molecular events that result in apoptosis can lead to observable and characteristic morphological changes to a cell, including membrane blebbing, cell volume shrinkage, chromosomal DNA condensation and fragmentation, and mRNA decay. A common method to identify cells, including T cells, undergoing apoptosis is to expose cells to a fluorophore-conjugated protein (Annexin V). Annexin V is commonly used to detect apoptotic cells by its ability to bind to phosphatidylserine on the outer leaflet of the plasma membrane, which is an early indicator that the cell is undergoing the process of apoptosis.

As used herein, the term “B cell” (alternatively “B lymphocyte”) refers to a type of white blood cell of the lymphocyte subtype. B cells function in the humoral immunity component of the adaptive immune system by secreting antibodies. B cells also present antigen and secrete cytokines. B cells, unlike the other two classes of lymphocytes, T cells and natural killer cells, express B cell receptors (BCRs) on their cell membrane. BCRs allow the B cell to bind to a specific antigen, against which it will initiate an antibody response.

As used herein, the term “binds to immobilized IL-27,” refers to the ability of an antibody of the disclosure to bind to IL-27, for example, expressed on the surface of a cell or which is attached to a solid support.

As used herein, the term “bispecific” or “bifunctional antibody” refers to an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, (1990) Clin. Exp. Immunol. 79:315-321; Kostelny et al., (1992) J. Immunol. 148:1547-1553.

Traditionally, the recombinant production of bispecific antibodies is based on the co-expression of two immunoglobulin heavy-chain/light-chain pairs, where the two heavy chain/light chain pairs have different specificities (Milstein and Cuello, (1983) Nature 305:537-539). Antibody variable domains with the desired binding specificities (antibody-antigen combining sites) can be fused to immunoglobulin constant domain sequences. The fusion of the heavy chain variable region is preferably with an immunoglobulin heavy-chain constant domain, including at least part of the hinge, CH2, and CH3 regions. For further details of illustrative currently known methods for generating bispecific antibodies see, e.g., Suresh et al., (1986) Methods Enzymol. 121:210; PCT Publication No. WO 96/27011; Brennan et al., (1985) Science 229:81; Shalaby et al., J. Exp. Med. (1992) 175:217-225; Kostelny et al., (1992) J. Immunol. 148 (5): 1547-1553; Hollinger et al., (1993) Proc. Natl. Acad. Sci. USA 90:6444-6448; Gruber et al., (1994) J. Immunol. 152:5368; and Tutt et al., (1991) J. Immunol. 147:60. Bispecific antibodies also include cross-linked or heteroconjugate antibodies. Heteroconjugate antibodies may be made using any convenient cross-linking methods. Suitable cross-linking agents are well known in the art, and are disclosed in U.S. Pat. No. 4,676,980, along with a number of cross-linking techniques.

Various techniques for making and isolating bispecific antibody fragments directly from recombinant cell culture have also been described. For example, bispecific antibodies have been produced using leucine zippers. See, e.g., Kostelny et al. (1992) J Immunol 148 (5): 1547-1553. The leucine zipper peptides from the Fos and Jun proteins may be linked to the Fab′ portions of two different antibodies by gene fusion. The antibody homodimers may be reduced at the hinge region to form monomers and then re-oxidized to form the antibody heterodimers. This method can also be utilized for the production of antibody homodimers. The “diabody” technology described by Hollinger et al. (1993) Proc Natl Acad Sci USA 90:6444-6448 has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (scFv) dimers has also been reported. See, e.g., Gruber et al. (1994) J Immunol 152:5368. Alternatively, the antibodies can be “linear antibodies” as described in, e.g., Zapata et al. (1995) Protein Eng. 8 (10): 1057-1062. Briefly, these antibodies comprise a pair of tandem Fd segments (VH-CH1-VH-CH1) which form a pair of antigen binding regions. Linear antibodies can be bispecific or monospecific.

Antibodies with more than two valencies (e.g., trispecific antibodies) are contemplated and described in, e.g., Tutt et al. (1991) J Immunol 147:60.

The disclosure also embraces variant forms of multi-specific antibodies such as the dual variable domain immunoglobulin (DVD-Ig) molecules described in Wu et al. (2007) Nat Biotechnol 25 (11): 1290-1297. The DVD-Ig molecules are designed such that two different light chain variable domains (VL) from two different parent antibodies are linked in tandem directly or via a short linker by recombinant DNA techniques, followed by the light chain constant domain. Similarly, the heavy chain comprises two different heavy chain variable domains (VH) linked in tandem, followed by the constant domain CH1 and Fc region. Methods for making DVD-Ig molecules from two parent antibodies are further described in, e.g., PCT Publication Nos. WO 08/024188 and WO 07/024715. In some aspects, the bispecific antibody is a Fabs-in-Tandem immunoglobulin, in which the light chain variable region with a second specificity is fused to the heavy chain variable region of a whole antibody. Such antibodies are described in, e.g., International Patent Application Publication No. WO 2015/103072.

As used herein, “cancer antigen” or “tumor antigen” refers to (i) tumor-specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other type of antigen or antigen-presenting cell or material that is associated with a cancer.

As used herein, the term “cancer-specific immune response” refers to the immune response induced by the presence of tumors, cancer cells, or cancer antigens. In certain aspects, the response includes the proliferation of cancer antigen specific lymphocytes. In certain aspects, the response includes expression and upregulation of antibodies and T-cell receptors and the formation and release of lymphokines, chemokines, and cytokines. Both innate and acquired immune systems interact to initiate antigenic responses against the tumors, cancer cells, or cancer antigens. In certain aspects, the cancer-specific immune response is a T cell response.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The anti-IL-27 antibodies described herein can be used to treat patients who have, who are suspected of having, or who may be at high risk for developing any type of cancer, including renal carcinoma or melanoma, or any viral disease. Exemplary carcinomas include those forming from tissue of the cervix, lung, prostate, breast, head and neck, colon and ovary. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

As used herein, the term “CD112R” refers to a member of poliovirus receptor-like proteins and is a co-inhibitory receptor for human T cells. CD112R is an inhibitory receptor primarily expressed by T cells and NK cells and competes for CD112 binding with the activating receptor CD226. The interaction of CD112 with CD112R is of higher affinity than with CD226 and thereby effectively regulates CD226 mediated cell activation. Anti-CD112R antagonists that block the interaction with CD112 limit inhibitory signaling directly downstream of CD112R while simultaneously promoting greater immune cell activation by increasing CD226 interactions with CD112. As used herein the term “CD112R inhibitor” refers to an agent that disrupts, blocks or inhibits the biological function or activity of CD112R.

As used herein, the term “CD137” (alternatively “4-1BB”) refers to a member of the tumor necrosis factor (TNF) receptor superfamily. 4-1BB is a co-stimulatory immune checkpoint molecule, primarily for activated T cells. Crosslinking of CD137 enhances T cell proliferation, IL-2 secretion, survival and cytolytic activity. As used herein, the term “4-1BB agonist” refers to an agent that stimulates, induces or increases one or more function of 4-1BB. An exemplary 4-1BB agonist is Utomilumab (PF-05082566), a fully human IgG2 monoclonal antibody that targets this 4-1BB to stimulate T cells.

As used herein, the term “CD161” (alternatively known as Killer cell lectin-like receptor subfamily B, member 1 (KLRB1); NK1.1, or NKR-P1A) refers to a member of the C-type lectin superfamily. CD161 is a marker of T cells and CD161 expression has been associated with T cell infiltration into the tumor microenvironment for a number of different cancer types. CD161 is further described in Fergusson et al., (2014) Cell Reports 9 (3): 1075-1088, which is incorporated herein by reference it its entirety.

As used herein, the term “IL-27” or “interleukin 27” refers to the IL-27 cytokine. IL-27 is related to the IL-6/IL-12 cytokine families, and is a heterodimeric cytokine that comprises a first subunit known as Epstein-Barr Virus Induced Gene 3 (EBI3; also known as IL-27 subunit β and IL-27B) and a second subunit known as IL-27p28 (also known as IL30, IL-27 subunit a and IL-27A). IL-27 is predominantly synthesized by activated antigen-presenting cells including monocytes, endothelial cells and dendritic cells (Jankowski et al. (2010) Arch Immunol. Ther. Exp. 58:417-425, Diakowski et al. (2013) Adv. Clin. Exp. Med. (2013) 22 (5): 683-691). Although IL-27 can have proinflammatory effects, many studies suggest an important role of IL-27 as an immunosuppressive agent (Shimizu et al. (2006) J. Immunol. 176:7317-7324, Hisada et al. (2004) Cancer Res. 64:1152-1156, Diakowski (2013) supra). Although it was initially described as a factor promoting the initiation of Th1 responses, IL-27 was later found to play a major T-cell suppressive function by limiting Th1 responses, inhibiting Th2 and Th17 cell differentiation, and regulating the development of Tr1 and other T regulatory cell populations (Dietrich et al. (2014) J. Immunol. 192:5382-5389). In addition to its role as an immunoregulator, IL-27 also regulates angiogenesis, hematopoiesis, and osteocalstogenesis (Id.).

IL-27 signals through a heterodimeric type I cytokine receptor (the IL-27 receptor or IL-27R) that comprises a first subunit known as WSX-1 (also known as IL-27 receptor subunit a, IL-27RA, T-Cell Cytokine Receptor Type 1 (TCCR), and Cytokine Receptor-Like 1 (CRL1)) and a second subunit known as gp130 (also known as Interleukin-6 Signal Transducer (IL6ST), Interleukin-6 Receptor Subunit β (IL-6RB), and Oncostatin M Receptor). gp130 is also a receptor subunit for the IL-6 family cytokines (Liu et al. (2008) Scan. J. Immunol. 68:22-299, Diakowski (2013) supra). IL-27 signaling through IL-27R activates multiple signaling cascades, including the JAK-STAT and p38 MAPK pathways.

EBI3 is also believed to have biological functions independent of p28 or the IL-27 heterodimer. For example, EBI3 also interacts with p35 to form the heterodimeric cytokine IL-35 (Yoshida et al. (2015) Annu. Rev Immunol. 33:417-43) and has been shown to be selectively overexpressed in certain cell types without a corresponding increase in p28 or IL-27 (Larousserie et al. (2005) Am. J. Pathol. 166 (4): 1217-28).

An amino acid sequence of an exemplary human EBI3 protein is provided in SEQ ID NO: 1 (NCBI Reference Sequence: NP_005746.2; N-mtpqlllalvlwascppcsgrkgppaaltlprvqcrasrypiavdcswtlppapnstspvsfiatyrlgmaarghswpclqqtptstsctit dvqlfsmapyvlnvtavhpwgssssfvpfitehiikpdppegvrlsplaerqlqvqweppgswpfpeifslkywirykrqgaarfhrv gpieatsfilravrpraryyvqvaaqdltdygelsdwslpatatmslgk-C). An amino acid sequence of an exemplary human p28 protein is provided in SEQ ID NO: 2 (NCBI Reference Sequence: NP_663634.2; N-mgqtagdlgwrlsllllplllvqagvwgfprppgrpqlslqelrreftvslhlarkllsevrgqahrfaeshlpgvnlyllplgeqlpdvsltf qawrrlsdperlcfisttlqpfhallgglgtqgrwtnmermqlwamrldlrdlqrhlrfqvlaagfnlpeeeeeeeeeeeeerkgllpgalg salqgpaqvswpqllstyrllhslelvlsravrellllskaghsvwplgfptlspqp-C). An amino acid sequence of an exemplary human WSX1 protein is provided in SEQ ID NO: 3 (NCBI Reference Sequence: NP_004834.1; N-mrggrgapfwlwplpklallpllwvlfqrtrpqgsagplqcygvgplgdlncsweplgdlgapselhlqsqkyrsnktqtvavaagrs wvaipreqltmsdkllvwgtkagqplwppvfvnletqmkpnaprlgpdvdfseddpleatvhwapptwpshkvlicqfhyrrcqea awtllepelktipltpveiqdlelatgykvygrcrmekeedlwgewspilsfqtppsapkdvwvsgnlcgtpggeeplllwkapgpcv qvsykvwfwvggrelspegitcccslipsgaewarvsavnatswepltnlslvcldsasaprsvavssiagstellvtwqpgpgepleh vvdwardgdpleklnwvrlppgnlsallpgnftvgvpyritvtavsasglasassvwgfreelaplvgptlwrlqdappgtpaiawgev prhqlrghlthytlcaqsgtspsvcmnvsgntqsvtlpdlpwgpcelwvtastiagqgppgpilrlhlpdntlrwkvlpgilflwglfllgc glslatsgrcyhlrhkvlprwvwekvpdpansssgqphmeqvpeaqplgdlpileveemepppvmessqpaqatapldsgyekhf lptpeelgllgpprpqvla-C). An amino acid sequence of an exemplary human gp 130 protein is provided in SEQ ID NO: 4 (NCBI Reference Sequence: NP_002175.2; N-mltlqtwlvqalfiflttestgelldpcgyispespvvqlhsnftavcvlkekcmdyfhvnanyivwktnhftipkeqytiinrtassvtftd iaslniqltcniltfgqleqnvygitiisglppekpknlscivnegkkmrcewdggrethletnftlksewathkfadckakrdtptsctvd ystvyfvnievwveaenalgkvtsdhinfdpvykvkpnpphnlsvinseelssilkltwtnpsiksviilkyniqyrtkdastwsqippe dtastrssftvqdlkpfteyvfrircmkedgkgywsdwseeasgityedrpskapsfwykidpshtqgyrtvqlvwktlppfeangkil dyevtltrwkshlqnytvnatkltvnltndrylatltvrnlvgksdaavltipacdfqathpvmdlkafpkdnmlwvewttpresvkkyi lewcvlsdkapcitdwqqedgtvhrtylrgnlaeskcylitvtpvyadgpgspesikaylkqappskgptvrtkkvgkneavlewdql pvdvqngfirnytifyrtiignetavnvdsshteytlssltsdtlymvrmaaytdeggkdgpeftfttpkfaqgeieaivvpvclafllttllg vlfcfnkrdlikkhiwpnvpdpskshiaqwsphtpprhnfnskdqmysdgnftdvsvveieandkkpfpedlksldlfkkekinteg hssgiggsscmsssrpsisssdenessqntsstvqystvvhsgyrhqvpsvqvfsrsestqplldseerpedlqlvdhvdggdgilprqq yfkqncsqhesspdishferskqvssvneedfvrlkqqisdhisqscgsgqmkmfqevsaadafgpgtegqverfetvgmeaatdeg mpksylpqtvrqggympq-C).

As used herin, the term “combined positive score” or “CPS” refers to the number of positive tumor cells, lymphocytes and macrophages, divided by the total number of viable tumor cells multiplied by 100.

As used herein the term “compete”, when used in the context of antigen-binding proteins (e.g., immunoglobulins, antibodies, or antigen-binding fragments thereof) that compete for binding to the same epitope, refers to a interaction between antigen-binding proteins as determined by an assay (e.g., a competitive binding assay; a cross-blocking assay), wherein a test antigen-binding protein (e.g., a test antibody) inhibits (e.g., reduces or blocks) specific binding of a reference antigen-binding protein (e.g., a reference antibody) to a common antigen (e.g., IL-27 or a fragment thereof).

A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence. Polypeptides derived from another peptide may have one or more mutations relative to the starting polypeptide, e.g., one or more amino acid residues which have been substituted with another amino acid residue or which has one or more amino acid residue insertions or deletions.

A polypeptide can comprise an amino acid sequence which is not naturally occurring. Such variants necessarily have less than 100% sequence identity or similarity with the starting molecule. In certain aspects, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule.

In certain aspects, the antibodies of the disclosure are encoded by a nucleotide sequence. Nucleotide sequences of the disclosure can be useful for a number of applications, including: cloning, gene therapy, protein expression and purification, mutation introduction, DNA vaccination of a host in need thereof, antibody generation for, e.g., passive immunization, PCR, primer and probe generation, and the like.

It will also be understood by one of ordinary skill in the art that the antibodies suitable for use in the methods disclosed herein may be altered such that they vary in sequence from the naturally occurring or native sequences from which they were derived, while retaining the desirable activity of the native sequences. For example, nucleotide or amino acid substitutions leading to conservative substitutions or changes at “non-essential” amino acid residues may be made. Mutations may be introduced by standard techniques, such as site-directed mutagenesis and PCR-mediated mutagenesis.

The antibodies suitable for use in the methods disclosed herein may comprise conservative amino acid substitutions at one or more amino acid residues, e.g., at essential or non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a binding polypeptide is preferably replaced with another amino acid residue from the same side chain family. In certain aspects, a string of amino acids can be replaced with a structurally similar string that differs in order and/or composition of side chain family members. Alternatively, in certain aspects, mutations may be introduced randomly along all or part of a coding sequence, such as by saturation mutagenesis, and the resultant mutants can be incorporated into binding polypeptides of the disclosure and screened for their ability to bind to the desired target.

As used herein, the term antigen “cross-presentation” refers to presentation of exogenous protein antigens to T cells via MHC class I and class II molecules on APCs.

As used herein, the term “cross-reacts” refers to the ability of an antibody of the disclosure to bind to IL-27 from a different species. For example, an antibody of the present disclosure which binds human IL-27 may also bind another species of IL-27. As used herein, cross-reactivity is measured by detecting a specific reactivity with purified antigen in binding assays (e.g., SPR, ELISA) or binding to, or otherwise functionally interacting with, cells physiologically expressing IL-27. Methods for determining cross-reactivity include standard binding assays as described herein, for example, by Biacore™ surface plasmon resonance (SPR) analysis using a Biacore™ 2000 SPR instrument (Biacore AB, Uppsala, Sweden), or flow cytometric techniques.

As used herein, the term “cytotoxic T lymphocyte (CTL) response” refers to an immune response induced by cytotoxic T cells. CTL responses are mediated primarily by CD8+ T cells.

As used herein, the term “dendritic cell” or “DC” refers to type of antigen-presenting cells that are bone marrow (BM)-derived leukocytes and are the most potent type of antigen-presenting cells. DCs are capture and process antigens, converting proteins to peptides that are presented on major histocompatibility complex (MHC) molecules recognized by T cells. DCs are heterogeneous, e.g. myeloid and plasmacytoid DCs; although all DCs are capable of antigen uptake, processing and presentation to naive T cells, the DC subtypes have distinct markers and differ in location, migratory pathways, detailed immunological function and dependence on infections or inflammatory stimuli for their generation. During the development of an adaptive immune response, the phenotype and function of DCs play a role in initiating tolerance, memory, and polarized T-helper 1 (Th1), Th2 and Th17 differentiation.

As used herein, the term “dendritic cell activation” refers to the transition from immature to mature dendritic cell; and the activated dendritic cells encompass mature dendritic cells and dendritic cells in the process of the transition, wherein the expression of CD80 and CD86 that induce costimulatory signals are elevated by the activating stimuli. Mature human dendritic cells are cells that are positive for the expression of CD40, CD80, CD86, and HLA-class II (e.g., HLA-DR). An immature dendritic cell can be distinguished from a mature dendritic cell, for example, based on markers selected from the group consisting of CD80 and CD86. An immature dendritic cell is weakly positive and preferably negative for these markers, while a mature dendritic cell is positive. Discrimination of mature dendritic cells is routinely performed by those skilled in the art, and the respective markers described above and methods for measuring their expression are also well known to those skilled in the art.

As used herein, the term “EC₅₀” refers to the concentration of an antibody or an antigen-binding portion thereof, which induces a response, either in an in vitro or an in vivo assay, which is 50% of the maximal response, i.e., halfway between the maximal response and the baseline.

As used herein, the term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the disorder being treated and the general state of the patient's own immune system.

As used herein, the term “epitope” or “antigenic determinant” refers to a site on an antigen to which an immunoglobulin or antibody specifically binds. The term “epitope mapping” refers to a process or method of identifying the binding site, or epitope, of an antibody, or antigen binding fragment thereof, on its target protein antigen. Epitope mapping methods and techniques are provided herein. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from IL-27 are tested for reactivity with the given anti-IL-27 antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

Also encompassed by the present disclosure are antibodies that bind to an epitope on IL-27 which comprises all or a portion of an epitope recognized by the particular antibodies described herein (e.g., the same or an overlapping region or a region between or spanning the region).

Also encompassed by the present disclosure are antibodies that bind the same epitope and/or antibodies that compete for binding to human IL-27 with the antibodies described herein. Antibodies that recognize the same epitope or compete for binding can be identified using routine techniques. Such techniques include, for example, an immunoassay, which shows the ability of one antibody to block the binding of another antibody to a target antigen, i.e., a competitive binding assay. Competitive binding is determined in an assay in which the immunoglobulin under test inhibits specific binding of a reference antibody to a common antigen, such as IL-27. Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using I-125 label (see Morel et al., Mol. Immunol. 25 (1): 7 (1988)); solid phase direct biotin-avidin EIA (Cheung et al., Virology 176:546 (1990)); and direct labeled RIA. (Moldenhauer et al., Scand. J. Immunol. 32:77 (1990)). Typically, such an assay involves the use of purified antigen bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70% 70-75% or more.

Other techniques include, for example, epitope mapping methods, such as, x-ray analyses of crystals of antigen: antibody complexes which provides atomic resolution of the epitope and mass spectrometry combined with hydrogen/deuterium (H/D) exchange which studies the conformation and dynamics of antigen: antibody interactions. Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. The peptides are then regarded as leads for the definition of the epitope corresponding to the antibody used to screen the peptide library. For epitope mapping, computational algorithms have also been developed which have been shown to map conformational discontinuous epitopes.

As used herein, the term “Fc-mediated effector functions” or “Fc effector functions” refer to the biological activities of an antibody other than the antibody's primary function and purpose. For example, the effector functions of a therapeutic agnostic antibody are the biological activities other than the activation of the target protein or pathway. Examples of antibody effect functions include C1q binding and complement dependent cytotoxicity; Fc receptor binding; antibody-dependent cell-mediated cytotoxicity (ADCC); phagocytosis; down regulation of cell surface receptors (e.g., B cell receptor); lack of activation of platelets that express Fc receptor; and B cell activation. Many effector functions begin with Fc binding to an Fcγ receptor. In some aspects, the tumor antigen-targeting antibody has effector function, e.g., ADCC activity. In some aspects, a tumor antigen-targeting antibody described herein comprises a variant constant region having increased effector function (e.g. increased ability to mediate ADCC) relative to the unmodified form of the constant region.

As used herein, the term “Fc receptor” refers to a polypeptide found on the surface of immune effector cells, which is bound by the Fc region of an antibody. In some aspects, the Fc receptor is an Fcγ receptor. There are three subclasses of Fcγ receptors, FcγRI (CD64), FcγRII (CD32) and FγcRIII (CD16). All four IgG isotypes (IgG1, IgG2, IgG3 and IgG4) bind and activate Fc receptors FcγRI, FcγRIIA and FcγRIIIA. FcγRIIB is an inhibitory receptor, and therefore antibody binding to this receptor does not activate complement and cellular responses. FcγRI is a high affinity receptor that binds to IgG in monomeric form, whereas FcγRIIA and FcγRIIA are low affinity receptors that bind IgG only in multimeric form and have slightly lower affinity. The binding of an antibody to an Fc receptor and/or C1q is governed by specific residues or domains within the Fc regions. Binding also depends on residues located within the hinge region and within the CH2 portion of the antibody. In some aspects, the agonistic and/or therapeutic activity of the antibodies described herein is dependent on binding of the Fc region to the Fc receptor (e.g., FcγR). In some aspects, the agonistic and/or therapeutic activity of the antibodies described herein is enhanced by binding of the Fc region to the Fc receptor (e.g., FcγR).

A list of certain Fc receptor sequences employed in the instant disclosure is set forth as Table 1B below.

As used herein, the term “GBP5” or “Guanylate Binding Protein 5” refers to a member of the guanylate-binding proteins (GBPs). GBP5 has a variety of biological functions, and the encoded the encoded protein acts as an activator of NLRP3 inflammasome assembly and has a role in innate immunity and inflammation. (See, e.g., Li, Xiang et al. Frontiers in Genetics vol. 13 984615. 30 Sep. 2022, which is incorporated herein by reference in its entirety).

As used herein, the term “glycosylation pattern” is defined as the pattern of carbohydrate units that are covalently attached to a protein, more specifically to an immunoglobulin protein. A glycosylation pattern of a heterologous antibody can be characterized as being substantially similar to glycosylation patterns which occur naturally on antibodies produced by the species of the nonhuman transgenic animal, when one of ordinary skill in the art would recognize the glycosylation pattern of the heterologous antibody as being more similar to said pattern of glycosylation in the species of the nonhuman transgenic animal than to the species from which the CH genes of the transgene were derived.

As used herein, the term “human antibody” includes antibodies having variable and constant regions (if present) of human germline immunoglobulin sequences. Human antibodies of the disclosure can include amino acid residues not encoded by human germline immunoglobulin sequences (e.g., mutations introduced by random or site-specific mutagenesis in vitro or by somatic mutation in vivo) (See, e.g., Lonberg et al., (1994) Nature 368 (6474): 856-859); Lonberg, (1994) Handbook of Experimental Pharmacology 113:49-101; Lonberg & Huszar, (1995) Intern. Rev. Immunol. 13:65-93, and Harding & Lonberg, (1995) Ann. N.Y. Acad. Sci. 764:536-546). However, the term “human antibody” does not include antibodies in which CDR sequences derived from the germline of another mammalian species, such as a mouse, have been grafted onto human framework sequences (i.e. humanized antibodies).

As used herein, the term a “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

As used herein, the term “Interferon regulatory factor 1” or “IRF1” refers to a nuclear factor that binds and activates the promoters of type I interferon genes. IRF1 is an early-target gene downstream of IFNγ signaling and modulates IFNy-mediated gene induction. IRF-1, a member of the interferon regulatory factor (IRF) family. IRF-1 was first identified in 1988 as a transcription factor able to induce expression of the gene interferon β (IFN-β). See Dou, Lei et al. Human ImmunologyVol. 75,11 (2014): 1110-4, which is incorporated herein by reference in its entirety.

As used herein, the term “immune cell” refers to any cell of the human immune system. The term “immune cell” includes, without limitation, lymphocytes (e.g., T cells, B cells, and tumor infiltrating lymphocytest (TILs)), macrophages, basophils, eosinophils, neutrophils, monocytes, and natural killer (NK) cells.

The terms “inducing an immune response” and “enhancing an immune response” are used interchangeably and refer to the stimulation of an immune response (i.e., either passive or adaptive) to a particular antigen. The terms “induce” as used with respect to inducing CDC or ADCC refer to the stimulation of particular direct cell killing mechanisms.

As used herein, the term “immunogenic cell death” (alternatively known as “immunogenic apoptosis” refers to a cell death modality associated with the activation of one or more signaling pathways that induces the pre-mortem expression and emission of damaged-associated molecular pattern (DAMPs) molecules (e.g., adenosine triphosphate, ATP) from the tumor cell, resulting in the increase of immunogenicity of the tumor cell and the death of the tumor cell in an immunogenic manner (e.g., by phagocytosis). As used herein, the term “immunogenic cell death-inducing agent” refers to a chemical, biological, or pharmacological agent that induces an immunogenic cell death process, pathway, or modality.

As used herein, the terms “inhibits”, “reduces” or “blocks” (e.g., referring to inhibition or reduction of human IL-27-mediated phosphorylation of STAT1 and/or STAT3 in a cell) are used interchangeably and encompass both partial and complete inhibition/blocking. The inhibition/blocking of IL-27 reduces or alters the normal level or type of activity that occurs without inhibition or blocking. Inhibition and blocking are also intended to include any measurable decrease in the binding affinity of IL-27 when in contact with an anti-IL-27 antibody as compared to IL-27 not in contact with an anti-IL-27 antibody, e.g., inhibits binding of IL-27 by at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%.

As used herein, the terms “inhibits angiogenesis,” “diminishes angiogenesis,” and “reduces angiogenesis” refer to reducing the level of angiogenesis in a tissue to a quantity which is at least 10%, 15%, 20%, 25%, 30%, 35%, 40%, 45%, 50%, 55%, 60%, 65%, 70%, 75%, 80%, 85%, 90%, 95%, 99% or less than the quantity in a corresponding control tissue, and most preferably is at the same level which is observed in a control tissue.

As used herein, the term “inhibits growth” (e.g., referring to cells) is intended to include any measurable decrease in the growth of a cell, e.g., the inhibition of growth of a cell by at least about 10%, 20%, 30%, 40%, 50%, 60%, 70%, 80%, 90%, 99%, or 100%.

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment (such as treatment with a composition comprising an anti-IL-27 antibody).

The term “in vivo” refers to processes that occur in a living organism.

As used herein, the term “isolated antibody” is intended to refer to an antibody which is substantially free of other antibodies having different antigenic specificities (e.g., an isolated antibody that specifically binds to human IL-27 is substantially free of antibodies that specifically bind antigens other than IL-27). An isolated antibody that specifically binds to an epitope may, however, have cross-reactivity to other IL-27 proteins from different species. However, the antibody continues to display specific binding to human IL-27 in a specific binding assay as described herein. In addition, an isolated antibody is typically substantially free of other cellular material and/or chemicals. In some aspects, a combination of “isolated” antibodies having different IL-27 specificities is combined in a well-defined composition.

As used herein, the term “isolated nucleic acid molecule” refers to nucleic acids encoding antibodies or antibody portions (e.g., VH, VL, CDR3) that bind to IL-27, is intended to refer to a nucleic acid molecule in which the nucleotide sequences encoding the antibody or antibody portion are free of other nucleotide sequences encoding antibodies or antibody portions that bind antigens other than IL-27, which other sequences may naturally flank the nucleic acid in human genomic DNA. For example, a sequence selected from a sequence set forth in Table 1A corresponds to the nucleotide sequences comprising the heavy chain (VH) and light chain (VL) variable regions of anti-IL-27 antibody monoclonal antibodies described herein.

As used herein, “isotype” refers to the antibody class (e.g., IgM or IgG1) that is encoded by heavy chain constant region genes. In some aspects, a human monoclonal antibody of the disclosure is of the IgG1 isotype. In some aspects, a human monoclonal antibody of the disclosure is of the IgG2 isotype. In some aspects, a human monoclonal antibody of the disclosure is of the IgG3 isotype. In some aspects, a human monoclonal antibody of the disclosure is of the IgG4 isotype. As is apparent to a skilled artisan, identification of antibody isotypes (e.g., IgG1, IgG2, IgG3, IgG4, IgM, IgA1 IgA2, IgD, and IgE) is routine in the art and commonly involves a combination of sequence alignments with known antibodies, published Fc variant sequences and conserved sequences.

As used herein, the term “isotype switching” refers to the phenomenon by which the class, or isotype, of an antibody changes from one Ig class to one of the other Ig classes.

As used herein the term “KD” or “K_{D}” refers to the equilibrium dissociation constant of a binding reaction between an antibody and an antigen. The value of K_{D} is a numeric representation of the ratio of the antibody off-rate constant (kd) to the antibody on-rate constant (ka). The value of K_{D} is inversely related to the binding affinity of an antibody to an antigen. The smaller the K_{D} value the greater the affinity of the antibody for its antigen. Affinity is the strength of binding of a single molecule to its ligand and is typically measured and reported by the equilibrium dissociation constant (K_{D}), which is used to evaluate and rank order strengths of bimolecular interactions.

As used herein, the term “kd” or “k_{d}” (alternatively “koff” or “k_{off}”) is intended to refer to the off-rate constant for the dissociation of an antibody from an antibody/antigen complex. The value of ka is a numeric representation of the fraction of complexes that decay or dissociate per second, and is expressed in units sec-1.

As used herein, the term “ka” or “kₐ” (alternatively “kon” or “kₒₙ”) is intended to refer to the on-rate constant for the association of an antibody with an antigen. The value of ka is a numeric representation of the number of antibody/antigen complexes formed per second in a 1 molar (1M) solution of antibody and antigen, and is expressed in units M⁻¹ sec⁻¹.

As used herein, the term “leukocyte” refers to a type of white blood cell involved in defending the body against infective organisms and foreign substances. Leukocytes are produced in the bone marrow. There are 5 main types of white blood cells, subdivided between 2 main groups: polymorphonuclear leukocytes (neutrophils, eosinophils, basophils) and mononuclear leukocytes (monocytes and lymphocytes).

As used herein, the term “lymphocytes” refers to a type of leukocyte or white blood cell that is involved in the immune defenses of the body. There are two main types of lymphocytes: B-cells and T-cells.

As used herein, the terms “linked,” “fused”, or “fusion”, are used interchangeably. These terms refer to the joining together of two more elements or components or domains, by whatever means including chemical conjugation or recombinant means. Methods of chemical conjugation (e.g., using heterobifunctional crosslinking agents) are known in the art.

As used herein, “local administration” or “local delivery,” refers to delivery that does not rely upon transport of the composition or agent to its intended target tissue or site via the vascular system. For example, the composition may be delivered by injection or implantation of the composition or agent or by injection or implantation of a device containing the composition or agent. Following local administration in the vicinity of a target tissue or site, the composition or agent, or one or more components thereof, may diffuse to the intended target tissue or site.

As used herein, “MHC molecules” refers to two types of molecules, MHC class I and MHC class II. MHC class I molecules present antigen to specific CD8+ T cells and MHC class II molecules present antigen to specific CD4+ T cells. Antigens delivered exogenously to APCs are processed primarily for association with MHC class II. In contrast, antigens delivered endogenously to APCs are processed primarily for association with MHC class I.

As used herein, the term “monoclonal antibody” refers to an antibody which displays a single binding specificity and affinity for a particular epitope. Accordingly, the term “human monoclonal antibody” refers to an antibody which displays a single binding specificity and which has variable and optional constant regions derived from human germline immunoglobulin sequences. In some aspects, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

As used herein, the term “monocyte” refers to a type of leukocyte and can differentiate into macrophages and dendritic cells to effect an immune response.

As used herein, the term “natural killer (NK) cell” refers to a type of cytotoxic lymphocyte. These are large, usually granular, non-T, non-B lymphocytes, which kill certain tumor cells and play an important role in innate immunity to viruses and other intracellular pathogens, as well as in antibody-dependent cell-mediated cytotoxicity (ADCC).

As used herein, the term “naturally occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally occurring.

As used herein, the term “nonswitched isotype” refers to the isotypic class of heavy chain that is produced when no isotype switching has taken place; the CH gene encoding the nonswitched isotype is typically the first CH gene immediately downstream from the functionally rearranged VDJ gene. Isotype switching has been classified as classical or non-classical isotype switching. Classical isotype switching occurs by recombination events which involve at least one switch sequence region in the transgene. Non-classical isotype switching may occur by, for example, homologous recombination between human σ_{μ} and human Σ_{μ} (δ-associated deletion). Alternative non-classical switching mechanisms, such as intertransgene and/or interchromosomal recombination, among others, may occur and effectuate isotype switching.

As used herein, the term “nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., Biol. Chem. 260:2605-2608, 1985; and Cassol et al, 1992; Rossolini et al, Mol. Cell. Probes 8:91-98, 1994). For arginine and leucine, modifications at the second base can also be conservative. The term nucleic acid is used interchangeably with gene, cDNA, and mRNA encoded by a gene.

Polynucleotides used herein can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

As used herein, “parenteral administration,” “administered parenterally,” and other grammatically equivalent phrases, refer to modes of administration other than enteral and topical administration, usually by injection, and include, without limitation, intravenous, intranasal, intraocular, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intracerebral, intracranial, intracarotid and intrasternal injection and infusion.

As used herein, the term “patient” includes human and other mammalian subjects that receive either prophylactic or therapeutic treatment.

As used herein, the term “PD-1 antagonist” refers to any chemical compound or biological molecule that inhibits the PD-1 signaling pathway or that otherwise inhibits PD-1 function in a cell (e.g. an immune cell). In some aspects, a PD-1 antagonist blocks binding of PD-L1 to PD-1 and/or PD-L2 to PD-1. In some aspects, the PD-1 antagonist specifically binds PD-1. In some aspects, the PD-1 antagonist specifically binds PD-L1.

The term “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (see, e.g., Berge et al. (1977) J Pharm Sci 66:1-19).

As used herein, the terms “polypeptide,” “peptide”, and “protein” are used interchangeably to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “preventing” when used in relation to a condition, refers to administration of a composition which reduces the frequency of, or delays the onset of, symptoms of a medical condition in a subject relative to a subject which does not receive the composition.

As used herein, the term “purified” or “isolated” as applied to any of the proteins (antibodies or fragments) described herein refers to a polypeptide that has been separated or purified from components (e.g., proteins or other naturally occurring biological or organic molecules) which naturally accompany it, e.g., other proteins, lipids, and nucleic acid in a prokaryote expressing the proteins. Typically, a polypeptide is purified when it constitutes at least 60 (e.g., at least 65, 70, 75, 80, 85, 90, 92, 95, 97, or 99) %, by weight, of the total protein in a sample.

As used herein, the term “Programmed Cell Death Protein 1” or “PD-1” refers to the Programmed Cell Death Protein 1 polypeptide, an immune-inhibitory receptor belonging to the CD28 family and is encoded by the PDCD1 gene in humans. Alternative names or synonyms for PD-1 include: PDCD1, PD1, CD279 and SLEB2. PD-1 is expressed predominantly on previously activated T cells, B cells, and myeloid cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. AAC51773.

As used herein, the term “Programmed Death Ligand-1” or “PD-L1” is one of two cell surface glycoprotein ligands for PD-1 (the other being PD-L2) that downregulates T cell activation and cytokine secretion upon binding to PD-1. Alternative names and synonyms for PD-L1 include: PDCD1L1, PDL1, B7H1, B7-4, CD274 and B7-H. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

PD-1 is known as an immune-inhibitory protein that negatively regulates TCR signals (Ishida, Y. et al. (1992) EMBO J. 11:3887-3895; Blank, C. et al. (Epub 2006 Dec. 29) Immunol. Immunother. 56 (5): 739-745). The interaction between PD-1 and PD-L1 can act as an immune checkpoint, which can lead to a decrease in T-cell receptor mediated proliferation (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1 or PD-L2; the effect is additive when the interaction of PD-1 with PD-L2 is blocked as well (Iwai et al. (2002) Proc. Nat'l. Acad. Sci. USA 99:12293-7; Brown et al. (2003) J. Immunol. 170:1257-66).

For several cancers, tumor survival and proliferation is sustained by tumor-mediated immune checkpoint modulation. This modulation can result in the disruption of anti-cancer immune system functions. For example, recent studies have indicated that the expression of immune checkpoint receptors ligands, such as PD-L1 or PD-L2, by tumor cells can downregulate immune system activity in the tumor microenvironment and promote cancer immune evasion, particularly by suppressing T cells. PD-L1 is abundantly expressed by a variety of human cancers (Dong et al., (2002) Nat Med 8:787-789). The receptor for PD-L1, PD-1, is expressed on lymphocytes (e.g., activated T cells) and is normally involved in down-regulating the immune system and promoting self-tolerance, particularly by suppressing T cells. However, when PD-1 receptors expressed on T cells bind to cognate PD-L1 ligands on tumor cells, the resulting T cell suppression contributes to an impaired immune response against the tumor (e.g., a decrease in tumor infiltrating lymphocytes or the establishment of immune evasion by cancer cells).

In large sample sets of e.g. ovarian, renal, colorectal, pancreatic, liver cancers and melanoma, it was shown that PD-L1 expression correlated with poor prognosis and reduced overall survival irrespective of subsequent treatment (see e.g., Dong et al., (2002) Nat Med 8 (8): 793-800; Yang et al., (2008) Invest Ophthalmol Vis Sci 49 (6): 2518-2525; Ghebeh et al., (2006) Neoplasia 8:190-198; Hamanishi et al., (2007) Proc Nat Acad Sci USA 104:3360-3365; Thompson et al., (2006) Clin Genitourin Cancer 5:206-211; Nomi et al., (2005) Clin Cancer Res 11:2947-2953; Inman et al., (2007) Cancer 109:1499-1505; Shimauchi et al., (2007) Int J Cancer 121:2585-2590; Gao et al., (2009) Clin Cancer Res 15:971-979; Nakanishi et al., (2007) Cancer Immunol Immunother 56:1173-1182; Hino et al., (2010) Cancer 116 (7): 1757-1766). Similarly, PD-1 expression on tumor lymphocytes was found to mark dysfunctional T cells in breast cancer (Kitano et al., (2017) ESMO Open 2 (2): e000150) and melanoma (Kleffel et al., (2015) Cell 162 (6): 1242-1256). PD-1 antagonists, such as those that affect the function of the PD-1/PD-L1/PD-L2 signaling axis and/or disrupt the interaction between PD-1 and PD-L1 and/or PD-L2, for example, have been developed and represent a novel class of anti-tumor inhibitors that function via modulation of immune cell-tumor cell interaction.

As used herein, the term “recombinant host cell” (or simply “host cell”) is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

As used herein, the term “recombinant human antibody” includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences are encoded by the germline genes, but include subsequent rearrangements and mutations which occur, for example, during antibody maturation. As known in the art (see, e.g., Lonberg (2005) Nature Biotech. 23 (9): 1117-1125), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen may not have sequence identity with the original nucleic acid molecules, but instead will be substantially identical or similar (i.e., have at least 80% identity).

As used herein, the term “reference antibody” (used interchangeably with “reference mAb”) or “reference antigen-binding protein” refers to an antibody, or an antigen-binding fragment thereof, that binds to a specific epitope on IL-27 and is used to establish a relationship between itself and one or more distinct antibodies, wherein the relationship is the binding of the reference antibody and the one or more distinct antibodies to the same epitope on IL-27. As used herein, the term connotes an anti-IL-27 antibody that is useful in a test or assay, such as those described herein, (e.g., a competitive binding assay), as a competitor, wherein the assay is useful for the discovery, identification or development, of one or more distinct antibodies that bind to the same epitope.

As used herein, the terms “specific binding,” “selective binding,” “selectively binds,” and “specifically binds,” refer to antibody binding to an epitope on a predetermined antigen. Typically, the antibody binds with an equilibrium dissociation constant (K_{D}) of approximately less than 10⁻⁶ M, such as approximately less than 10⁻⁷, 10⁻⁸ M, 10⁻⁹ M or 10⁻¹⁰ M or even lower when determined by surface plasmon resonance (SPR) technology in a BIACORE 2000 instrument using recombinant human IL-27 as the analyte and the antibody as the ligand and binds to the predetermined antigen with an affinity that is at least two-fold greater than its affinity for binding to a non-specific antigen (e.g., BSA, casein) other than the predetermined antigen or a closely-related antigen. In certain aspects, an antibody that specifically binds to IL-27 binds with an equilibrium dissociation constant (K_{D}) of approximately less than 100 nM (10⁻⁷ M), optionally approximately less than 50 nM (5×10⁻⁸ M), optionally approximately less than 15 nM (1.5×10⁻⁸ M), optionally approximately less than 10 nM (10⁻⁸ M), optionally approximately less than 5 nM (5×10⁻⁹ M), optionally approximately less than 1 nM (10⁻⁹ M), optionally approximately less than 0.1 nM (10⁻¹⁰ M), optionally approximately less than 0.01 nM (10⁻¹¹ M), or even lower, when determined by surface plasmon resonance (SPR) technology in a BIACORE 2000 instrument using recombinant human IL-27 as the analyte and the antibody as the ligand, where binding to the predetermined antigen occurs with an affinity that is at least two-fold greater than the antibody's affinity for binding to a non-specific antigen (e.g., BSA, casein) other than the predetermined antigen or a closely-related antigen The phrases “an antibody recognizing an antigen” and “an antibody specific for an antigen” are used interchangeably herein with the term “an antibody which binds specifically to an antigen.”

As used herein, the term “STAT1 phosphorylation” refers to the phosphorylation of the Signal Transducer and Activator of Transcription 1 (STAT1) polypeptide, a transcription factor encoded by the STAT1 gene in humans. STAT molecules are phosphorylated by receptor associated kinases, that cause activation and dimerization by forming homo- or heterodimers which translocate to the nucleus to work as transcription factors. STAT1 can be activated (i.e., phosphorylated) in response to signaling via several ligands, including IL-27. IL-27 signaling through the IL-27R results in phosphorylation of STAT1 (pSTAT1). STAT1 has a key role in gene expression involved in survival of the cell, viability or pathogen response. Methods to determine STAT1 phosphorylation as a result of IL-27 signaling include, but are not limited to, flow cytometric analysis of cells labeled with antibodies that specifically recognize phosphorylated STAT1 (see e.g., Tochizawa et al., (2006) J Immunol Methods 313 (1-2): 29-37).

As used herein, the term “STAT3 phosphorylation” refers to the phosphorylation of the Signal Transducer and Activator of Transcription 3 (STAT3) polypeptide, a transcription factor encoded by the STAT3 gene in humans. STAT3 mediates the expression of a variety of genes in response to cell stimuli, and thus plays a key role in many cellular processes such as cell growth and apoptosis. Methods to determine STAT3 phosphorylation as a result of IL-27 signaling include, but are not limited to, analysis of cells or cell extracts labeled with antibodies that specifically recognize phosphorylated STAT3 (see e.g., Fursov et al., (2011) Assay Drug Dev Technol 9 (4): 420-429).

As used herein, the term “subject” includes any human or non-human animal. For example, the methods and compositions of the present disclosure can be used to treat a subject with an immune disorder. The term “non-human animal” includes all vertebrates, e.g., mammals and non-mammals, such as non-human primates, sheep, dog, cow, chickens, amphibians, reptiles, etc.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial homology exists when the segments will hybridize under selective hybridization conditions, to the complement of the strand.

The percent identity between two sequences is a function of the number of identical positions shared by the sequences (i.e., % homology=# of identical positions/total # of positions ×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48): 444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences of the present disclosure can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules of the disclosure. BLAST protein searches can be performed with the XBLAST program, score=50, wordlength=3 to obtain amino acid sequences homologous to the protein molecules of the disclosure. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used. See http://www.ncbi.nlm.nih.gov.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

The nucleic acid compositions of the present disclosure, while often in a native sequence (except for modified restriction sites and the like), from either cDNA, genomic or mixtures thereof may be mutated, in accordance with standard techniques to provide gene sequences. For coding sequences, these mutations, may affect amino acid sequence as desired. In particular, DNA sequences substantially homologous to or derived from native V, D, J, constant, switches and other such sequences described herein are contemplated (where “derived” indicates that a sequence is identical or modified from another sequence).

As used herein, the term “STING” (alternatively TMEM173) refers to the Stimulator of Interferon Genes, a protein that functions both as a direct cytosolic DNA sensor and as an adaptor protein. In humans, STING is encoded by the TMEM173 gene. STING plays an important role in innate immunity. STING induces type I interferon production when cells are infected with intracellular pathogens, such as viruses, mycobacteria and intracellular parasites. Type I interferon, mediated by STING, protects infected cells and nearby cells from local infection by binding to the same cell that secretes it and nearby cells. An exemplary amino acid sequence for STING is provided by the NCBI Genbank database under the accession number NP_001288667.

The term “T cell” refers to a type of white blood cell that can be distinguished from other white blood cells by the presence of a T cell receptor on the cell surface. There are several subsets of T cells, including, but not limited to, T helper cells (a.k.a. T_{H} cells or CD4⁺ T cells) and subtypes, including T_{H}1, T_{H}2, T_{H}3, T_{H}17, T_{H}9, and THE cells, cytotoxic T cells (a.k.a Tc cells, CD8⁺ T cells, cytotoxic T lymphocytes, T-killer cells, killer T cells), memory T cells and subtypes, including central memory T cells (T_{CM} cells), effector memory T cells (T_{EM} and T_{EMRA} cells), and resident memory T cells (T_{RM} cells), regulatory T cells (a.k.a. T_{reg} cells or suppressor T cells) and subtypes, including CD4⁺ FOXP3⁺ T_{reg} cells, CD4⁺FOXP3⁻ T_{reg} cells, Tr1 cells, Th3 cells, and T_{reg} 17 cells, natural killer T cells (a.k.a. NKT cells), mucosal associated invariant T cells (MAITs), and gamma delta T cells (γδ T cells), including Vγ9/Vδ2 T cells. Any one or more of the aforementioned or unmentioned T cells may be the target cell type for a method of use of the disclosure.

As used herein, the term “T cell-mediated response” refers to any response mediated by T cells, including, but not limited to, effector T cells (e.g., CD8⁺ cells) and helper T cells (e.g., CD4⁺ cells). T cell mediated responses include, for example, T cell cytotoxicity and proliferation.

As used herein, the terms “therapeutically effective amount” or “therapeutically effective dose,” or similar terms used herein are intended to mean an amount of an agent (e.g., an anti-IL-27 antibody or an antigen-binding fragment thereof) that will elicit the desired biological or medical response (e.g., an improvement in one or more symptoms of a cancer).

As used herein, the term “TAM receptor” refers to the TAM receptor protein tyrosine kinases (TYRO3, AXL and MER). TAM receptors are involved in the regulation of immune system homeostasis. In a cancer setting, TAM receptors have a dual regulatory role, controlling the initiation and progression of tumor development and, at the same time, the associated anti-tumor responses of diverse immune cells. Further description of TAM receptors is found in Paolino and Penninger (2016) Cancers 8 (97): doi: 10.3390/cancers8100097). As used herein, the term “TAM receptor inhibitor” or “TAM inhibitor” refers to an agent that inhibits, blocks or reduces the function or activity of a TAM receptor.

As used herein, the term “TIGIT” or “T-cell immunoreceptor with Ig and ITIM domains” refers to any native TIGIT from any vertebrate source, including mammals such as primates (e.g., humans) and rodents (e.g., mice and rats), unless otherwise indicated. TIGIT is also known in the art as DKFZp667A205, FLJ39873, V-set and immunoglobulin domain-containing protein 9, V-set and transmembrane domain-containing protein 3, VSIG9, VSTM3, and WUCAM. The term also encompasses naturally occurring variants of TIGIT, e.g., splice variants or allelic variants. The amino acid sequence of an exemplary human TIGIT may be found under UniProt Accession Number Q495A1.

The terms “treat,” “treating,” and “treatment,” as used herein, refer to therapeutic or preventative measures described herein. The methods of “treatment” employ administration to a subject, in need of such treatment, a human antibody of the present disclosure, for example, a subject in need of an enhanced immune response against a particular antigen or a subject who ultimately may acquire such a disorder, in order to prevent, cure, delay, reduce the severity of, or ameliorate one or more symptoms of the disorder or recurring disorder, or in order to prolong the survival of a subject beyond that expected in the absence of such treatment.

As used herein, the term “tumor microenvironment” (alternatively “cancer microenvironment”; abbreviated TME) refers to the cellular environment or milieu in which the tumor or neoplasm exists, including surrounding blood vessels as well as non-cancerous cells including, but not limited to, immune cells, fibroblasts, bone marrow-derived inflammatory cells, and lymphocytes. Signaling molecules and the extracellular matrix also comprise the TME. The tumor and the surrounding microenvironment are closely related and interact constantly. Tumors can influence the microenvironment by releasing extracellular signals, promoting tumor angiogenesis and inducing peripheral immune tolerance, while the immune cells in the microenvironment can affect the growth and evolution of tumor cells.

As used herein, the term “tumor proportion score” (TPS) refers to the number of positive tumor cells divided by the total number of viable tumor cells multiplied by 100%.

As used herein, the term “vector” is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the present disclosure is intended to include such other forms of expression vectors, such as viral vectors (e.g., replication defective retroviruses, adenoviruses and adeno-associated viruses), which serve equivalent functions.

Unless otherwise defined, all technical and scientific terms used herein have the same meaning as commonly understood by one of ordinary skill in the art to which this disclosure pertains. Preferred methods and materials are described below, although methods and materials similar or equivalent to those described herein can also be used in the practice or testing of the presently disclosed methods and compositions. All publications, patent applications, patents, and other references mentioned herein are incorporated by reference in their entirety.

### II. Methods of the Disclosure

Some aspects of the present disclosure are directed to methods of identifying a subject suitable for a therapy comprising an IL-27 inhibiting agent, e.g., an anti-IL-27 antibody disclosed herein. The present disclosure surprisingly finds that expression of various biomarkers can be indicative of responsiveness of a patient to an IL-27 inhibiting agent, e.g., an anti-IL-27 antibody disclosed herein. In some aspects, a patient suitable for a therapy comprising an IL-27 inhibiting agent, e.g., an anti-IL-27 antibody disclosed herein, is identified as having a tumor comprising tumor cells that express WSX-1, referred to herein as a “WSX-1-positive tumor.” In some aspects, a patient suitable for a therapy comprising an IL-27 inhibiting agent, e.g., an anti-IL-27 antibody disclosed herein, is identified as having a tumor comprising one or more immune cells within the tumor that express IL-27.

### A. WSX-1-Positive Tumors

Some aspects of the present disclosure are directed to methods for treating a tumor in a subject comprising administering an IL-27 inhibiting agent to the subject, wherein the tumor is identified as being a WSX-1-positive tumor. Some aspects of the present disclosure are directed to methods for treating a tumor in a subject in need thereof, comprising: (i) identifying a subject having a WSX-1-positive tumor; and (ii) administering to the subject an IL-27 inhibiting agent. In some aspects, the WSX-1-positive tumor is identified by detecting WSX-1 expression in a tumor sample obtained from the subject.

Some aspects of the present disclosure are directed to methods for identifying a human subject afflicted with a tumor suitable for treatment with an IL-27 inhibiting agent, the method comprising detecting WSX-1 expression in a tumor sample obtained from the subject. In some aspects, the method further comprises administering an IL-27 inhibiting agent to the subject identified as having a WSX-1-positive tumor.

In some aspects, the tumor sample obtained from the subject is a tumor tissue biopsy. In some aspects, the tumor sample obtained from the subject is a tumor tissue biopsy. In some aspects, the tumor sample obtained from the subject comprises tumor cells, tumor infiltrating lymphocytes, or both. In some aspects, at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, or at least about 50% of cells in the tumor sample express WSX-1. In some aspects, at least about 1% of cells in the tumor sample express WSX-1. In some aspects, at least about 2% of cells in the tumor sample express WSX-1. In some aspects, at least about 3% of cells in the tumor sample express WSX-1. In some aspects, at least about 4% of cells in the tumor sample express WSX-1. In some aspects, at least about 5% of cells in the tumor sample express WSX-1. In some aspects, at least about 10% of cells in the tumor sample express WSX-1. In some aspects, at least about 15% of cells in the tumor sample express WSX-1. In some aspects, at least about 20% of cells in the tumor sample express WSX-1. In some aspects, at least about 25% of cells in the tumor sample express WSX-1. In some aspects, at least about 30% of cells in the tumor sample express WSX-1. In some aspects, at least about 35% of cells in the tumor sample express WSX-1. In some aspects, at least about 40% of cells in the tumor sample express WSX-1. In some aspects, at least about 45% of cells in the tumor sample express WSX-1. In some aspects, at least about 50% of cells in the tumor sample express WSX-1. In some aspects, at least about 60% of cells in the tumor sample express WSX-1. In some aspects, at least about 70% of cells in the tumor sample express WSX-1. In some aspects, at least about 80% of cells in the tumor sample express WSX-1. In some aspects, at least about 90% of cells in the tumor sample express WSX-1. In some aspects, the cells in the tumor sample that express WSX-1 are tumor cells.

Any means of detecting WSX-1 expression can be used in the methods disclosed herein. In some aspects, expression of WSX-1 protein is detected. In some aspects, the WSX-1 expression is detected using an immunohistochemical (IHC) assay. In some aspects, the WSX-1 expression is detected using an automated IHC assay. In some aspects, the WSX-1 expression is detected by contacting the tumor sample with an antibody or an antigen-binding portion thereof that specifically binds human WSX-1. In some aspects, expression of WSX-1-encoding RNA is detected.

In some aspects, the tumor sample obtained from the subject is characterized by ≥60%, 65%, 70%, 75%, 80%, 85%, or 90% WSX-1 expression as measured by an assay (e.g., immunohistochemical (IHC) assay). In some aspects, the tumor sample obtained from the subject is characterized by a tumor proportion score (TPS) of ≥1% as measured by an assay. In some aspects, a sample is characterized by a tumor proportion score (TPS) of ≥5% as measured by an assay. In some aspects, the tumor sample obtained from the subject is characterized by a tumor proportion score (TPS) of ≥10% as measured by an assay. In some aspects, the tumor sample obtained from the subject is characterized by a tumor proportion score (TPS) of ≥25% as measured by an assay. In some aspects, the tumor sample obtained from the subject is characterized by a tumor proportion score (TPS) of ≥50% as measured by an assay. In some aspects, the tumor sample obtained from the subject obtained from the subject is characterized by a TPS≥60%, 65%, 70%, 75%, 80%, 85%, or 90% WSX-1 expression as measured by an assay (e.g., immunohistochemical (IHC) assay). In some aspects, the WSX-1 expression is assessed using a tumor proportional score. In some aspects, the TPS or CPS is at least 10%, at least 20%, at least 30%, at least 50% or at least 60%. In some aspects, the TPS or CPS is at least 10%. In some aspects, the TPS or CPS is at least 20%. In some aspects, the TPS or CPS is at least 30%. In some aspects, the TPS or CPS is at least 40%. In some aspects, the TPS or CPS is at least 50%. In some aspects, the TPS or CPS is at least 60%. In some aspects, the TPS is higher than the CPS for the same sample. In some aspects, the TPS or CPS is correlated with response to treatment with an IL-27 inhibiting agent.

The percent of cells that express WSX-1 in the tumor sample can be determined using any means. In some aspects, an IHC assay is used to label all WSX-1-expressing cells in a tumor sample; total cells in the tumor sample, e.g., a PPFE sample, are then counted; cells expressing WSX-1 are then counted; and the number of cells expressing WSX-1 is divided by the total number of cells in the tumor sample and multiplied by 100. In some aspects, the percent of cells expressing WSX-1 is an average across more than one PPFE sample prepared from a single tumor sample, e.g., tumor biopsy.

### B. IL-27-Positive Immune Cells

Some aspects of the present disclosure are directed to methods for treating a tumor in a subject comprising administering an IL-27 inhibiting agent to the subject, wherein one or more immune cells in a tumor sample obtained from the subject express IL-27. Some aspects of the present disclosure are directed to methods for treating a tumor in a subject in need thereof, comprising: (i) identifying a subject having a tumor wherein one or more immune cells in a tumor sample obtained from the subject express IL-27; and (ii) administering to the subject an IL-27 inhibiting agent.

Some aspects of the present disclosure are directed to methods for identifying a human subject afflicted with a tumor suitable for treatment with an IL-27 inhibiting agent, the method comprising detecting IL-27 expression in a tumor sample obtained from the subject. In some aspects, the method further comprises administering an IL-27 inhibiting agent to the subject identified as having a tumor sample comprising one or more immune cells that express IL-27.

In some aspects, the tumor sample obtained from the subject is a tumor tissue biopsy. In some aspects, the tumor sample obtained from the subject is a tumor tissue biopsy. In some aspects, the tumor sample obtained from the subject comprises tumor cells, tumor infiltrating lymphocytes, or both, the one or more immune cells in the tumor sample comprise macrophages

In some aspects, at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, or at least about 50% of the immune cells in the tumor sample express IL-27. In some aspects, at least about 1% of the immune cells in the tumor sample express IL-27. In some aspects, at least about 2% of the immune cells in the tumor sample express IL-27. In some aspects, at least about 3% of the immune cells in the tumor sample express IL-27. In some aspects, at least about 4% of the immune cells in the tumor sample express IL-27. In some aspects, at least about 5% of the immune cells in the tumor sample express IL-27. In some aspects, at least about 10% of the immune cells in the tumor sample express IL-27. In some aspects, at least about 15% of the immune cells in the tumor sample express IL-27. In some aspects, at least about 20% of the immune cells in the tumor sample express IL-27. In some aspects, at least about 25% of the immune cells in the tumor sample express IL-27. In some aspects, at least about 30% of the immune cells in the tumor sample express IL-27. In some aspects, at least about 35% of the immune cells in the tumor sample express IL-27. In some aspects, at least about 40% of the immune cells in the tumor sample express IL-27. In some aspects, at least about 45% of the immune cells in the tumor sample express IL-27. In some aspects, at least about 50% of the immune cells in the tumor sample express IL-27. In some aspects, at least about 60% of the immune cells in the tumor sample express IL-27. In some aspects, at least about 70% of the immune cells in the tumor sample express IL-27. In some aspects, at least about 80% of the immune cells in the tumor sample express IL-27. In some aspects, at least about 90% of immune cells in the tumor sample express IL-27.

Any means of detecting IL-27 expression can be used in the methods disclosed herein. In some aspects, expression of IL-27 protein is detected. In some aspects, the IL-27 expression is detected using an immunohistochemical (IHC) assay. In some aspects, the IL-27 expression is detected using an automated IHC assay. In some aspects, the v expression is detected by contacting the tumor sample with an antibody or an antigen-binding portion thereof that specifically binds human IL-27. In some aspects, expression of IL-27-encoding RNA is detected.

The percent of immune cells that express IL-27 in the tumor sample can be determined using any means. In some aspects, an IHC assay is used to label all IL-27-expressing cells in a tumor sample; the total number of immune cells in the tumor sample, e.g., a PPFE sample, are then counted; the cells expressing IL-27 are then counted; and the number of cells expressing IL-27 is divided by the total number of immune cells in the tumor sample and multiplied by 100. In some aspects, the percent of immune cells expressing IL-27 is an average across more than one PPFE sample prepared from a single tumor sample, e.g., tumor biopsy.

In some aspects, the tumor sample obtained from the subject is characterized by ≥60%, 65%, 70%, 75%, 80%, 85%, or 90% IL-27 expression as measured by an assay (e.g., immunohistochemical (IHC) assay). In some aspects, the tumor sample obtained from the subject is characterized by a tumor proportion score (TPS) of ≥1% as measured by an assay. In some aspects, a sample is characterized by a tumor proportion score (TPS) of ≥5% as measured by an assay. In some aspects, the tumor sample obtained from the subject is characterized by a tumor proportion score (TPS) of ≥10% as measured by an assay. In some aspects, the tumor sample obtained from the subject is characterized by a tumor proportion score (TPS) of ≥25% as measured by an assay. In some aspects, the tumor sample obtained from the subject is characterized by a tumor proportion score (TPS) of ≥50% as measured by an assay. In some aspects, the tumor sample obtained from the subject obtained from the subject is characterized by a TPS≥60%, 65%, 70%, 75%, 80%, 85%, or 90% IL-27 expression as measured by an assay (e.g., immunohistochemical (IHC) assay). In some aspects, the IL-27 expression is assessed using a tumor proportional score. In some aspects, the TPS or CPS is at least 10%, at least 20%, at least 30%, at least 50% or at least 60%. In some aspects, the TPS or CPS is at least 10%. In some aspects, the TPS or CPS is at least 20%. In some aspects, the TPS or CPS is at least 30%. In some aspects, the TPS or CPS is at least 40%. In some aspects, the TPS or CPS is at least 50%. In some aspects, the TPS or CPS is at least 60%. In some aspects, the TPS is higher than the CPS for the same sample. In some aspects, the TPS or CPS is correlated with response to treatment with an IL-27 inhibiting agent.

In some aspects, the antibody or an antigen binding portion thereof is administered at a dose of at least about 0.003 mg/kg to at least about 20 mg/kg. In some aspects, the antibody or antigen binding portion thereof specifically binds to an epitope comprising one or more amino acids of (i) amino acids 37 to 56 corresponding to SEQ ID NO: 2 (IL-27p28), (ii) amino acids 142 to 164 corresponding to SEQ ID NO: 2 (IL-27p28), or (iii) both (i) and (ii). In some aspects, the antibody or an antigen binding portion thereof is administered at a dose that is sufficient to maintain IC90 of pSTAT1 inhibition level, i.e., above about 0.7 ug/mL for the duration of the treatment, e.g., 28 days, 56 days, or 84 days.

In some aspects, the antibody or antigen binding portion thereof is administered at a dose of at least about 0.006 mg/kg to at least about 20 mg/kg, at least about 0.009 mg/kg to at least about 20 mg/kg, at least about 0.01 mg/kg to at least about 20 mg/kg, at least about 0.03 mg/kg to at least about 20 mg/kg, at least about 0.06 mg/kg to at least about 20 mg/kg, at least about 0.09 mg/kg to at least about 20 mg/kg, at least about 0.1 mg/kg to at least about 20 mg/kg, at least about 0.3 mg/kg to at least about 20 mg/kg, at least about 0.6 mg/kg to at least about 20 mg/kg, at least about 0.9 mg/kg to at least about 20 mg/kg, at least about 1 mg/kg to at least about 20 mg/kg, at least about 1 mg/kg to at least about 20 mg/kg, at least about 3 mg/kg to at least about 20 mg/kg, at least about 6 mg/kg to at least about 20 mg/kg, at least about 10 mg/kg to at least about 20 mg/kg, at least about 13 mg/kg to at least about 20 mg/kg, at least about 13 mg/kg to at least about 18 mg/kg, at least about 13 mg/kg to at least about 16 mg/kg, at least about 16 mg/kg to at least about 20 mg/kg, at least about 16 mg/kg to at least about 18 mg/kg, at least about 3 mg/kg to at least about 18 mg/kg, at least about 6 mg/kg to at least about 15 mg/kg, at least about 13 mg/kg to at least about 18 mg/kg, or at least about 10 mg/kg to at least about 15 mg/kg. In some aspects, the antibody or antigen binding portion thereof is administered at a dose of at least about 0.006 mg/kg to at least about 10 mg/kg, at least about 0.009 mg/kg to at least about 10 mg/kg, at least about 0.01 mg/kg to at least about 10 mg/kg, at least about 0.03 mg/kg to at least about 10 mg/kg, at least about 0.06 mg/kg to at least about 10 mg/kg, at least about 0.09 mg/kg to at least about 10 mg/kg, at least about 0.1 mg/kg to at least about 10 mg/kg, at least about 0.3 mg/kg to at least about 10 mg/kg, at least about 0.6 mg/kg to at least about 10 mg/kg, at least about 0.9 mg/kg to at least about 10 mg/kg, at least about 1 mg/kg to at least about 10 mg/kg, at least about 1 mg/kg to at least about 9 mg/kg, at least about 3 mg/kg to at least about 9 mg/kg, at least about 1 mg/kg to at least about 6 mg/kg, at least about 3 mg/kg to at least about 6 mg/kg, or at least about 1 mg/kg to at least about 3 mg/kg.

In some aspects, the antibody or antigen binding portion thereof is administered at a dose of at least about 0.003 mg/kg, at least about 0.006 mg/kg, at least about 0.009 mg/kg, at least about 0.01 mg/kg, at least about 0.03 mg/kg, at least about 0.06 mg/kg, at least about 0.09 mg/kg, at least about 0.1 mg/kg, at least about 0.3 mg/kg, at least about 0.6 mg/kg, at least about 0.9 mg/kg, at least about 1.0 mg/kg, at least about 2 mg/kg, at least about 3 mg/kg, at least about 4 mg/kg, at least about 5 mg/kg, at least about 6 mg/kg, at least about 7 mg/kg, at least about 8 mg/kg, at least about 9, or at least about 10 mg/kg. In some aspects, the antibody or antigen binding portion thereof is administered at a dose of at least about 11 mg/kg, at least about 12 mg/kg, at least about 13 mg/kg, at least about 14 mg/kg, at least about 15 mg/kg, at least about 16 mg/kg, at least about 17 mg/kg, at least about 18 mg/kg, at least about 19, or at least about 20 mg/kg. In some aspects, the antibody or antigen binding portion thereof is administered at a dose of at least about 0.003 mg/kg. In some aspects, the antibody or antigen binding portion thereof is administered at a dose of at least about 0.006 mg/kg. In some aspects, the antibody or antigen binding portion thereof is administered at a dose of at least about 0.009 mg/kg. In some aspects, the antibody or antigen binding portion thereof is administered at a dose of at least about 0.01 mg/kg. In some aspects, the antibody or antigen binding portion thereof is administered at a dose of at least about 0.03 mg/kg. In some aspects, the antibody or antigen binding portion thereof is administered at a dose of at least about 0.06 mg/kg. In some aspects, the antibody or antigen binding portion thereof is administered at a dose of at least about 0.09 mg/kg. In some aspects, the antibody or antigen binding portion thereof is administered at a dose of at least about 0.1 mg/kg. In some aspects, the antibody or antigen binding portion thereof is administered at a dose of at least about 0.3 mg/kg. In some aspects, the antibody or antigen binding portion thereof is administered at a dose of at least about 0.6 mg/kg. In some aspects, the antibody or antigen binding portion thereof is administered at a dose of at least about 0.9 mg/kg. In some aspects, the antibody or antigen binding portion thereof is administered at a dose of at least about 1.0 mg/kg. In some aspects, the antibody or antigen binding portion thereof is administered at a dose of at least about 2 mg/kg. In some aspects, the antibody or antigen binding portion thereof is administered at a dose of at least about 3 mg/kg. In some aspects, the antibody or antigen binding portion thereof is administered at a dose of at least about 4 mg/kg. In some aspects, the antibody or antigen binding portion thereof is administered at a dose of at least about 5 mg/kg. In some aspects, the antibody or antigen binding portion thereof is administered at a dose of at least about 6 mg/kg. In some aspects, the antibody or antigen binding portion thereof is administered at a dose of at least about 7 mg/kg. In some aspects, the antibody or antigen binding portion thereof is administered at a dose of at least about 8 mg/kg. In some aspects, the antibody or antigen binding portion thereof is administered at a dose of at least about 9. In some aspects, the antibody or antigen binding portion thereof is administered at a dose of at least about 10 mg/kg. In some aspects, the antibody or antigen binding portion thereof is administered at a dose of at least about 11 mg/kg. In some aspects, the antibody or antigen binding portion thereof is administered at a dose of at least about 12 mg/kg. In some aspects, the antibody or antigen binding portion thereof is administered at a dose of at least about 13 mg/kg. In some aspects, the antibody or antigen binding portion thereof is administered at a dose of at least about 14 mg/kg. In some aspects, the antibody or antigen binding portion thereof is administered at a dose of at least about 15 mg/kg. In some aspects, the antibody or antigen binding portion thereof is administered at a dose of at least about 16 mg/kg. In some aspects, the antibody or antigen binding portion thereof is administered at a dose of at least about 17 mg/kg. In some aspects, the antibody or antigen binding portion thereof is administered at a dose of at least about 18 mg/kg. In some aspects, the antibody or antigen binding portion thereof is administered at a dose of at least about 19 mg/kg. In some aspects, the antibody or antigen binding portion thereof is administered at a dose of at least about 20 mg/kg.

In some aspects, the antibody or antigen binding portion thereof is administered once about every week, once about every two weeks, once about every three weeks, once about every four weeks, once about every 6 weeks, once about every 8 weeks, or once about every 12 weeks. In some aspects, the antibody or antigen binding portion thereof is administered once about every four weeks.

In some aspects, the antibody or antigen binding portion thereof is administered at a dose of about 0.3 mg/kg once about every week. In some aspects, the antibody or antigen binding portion thereof is administered at a dose of about 1 mg/kg once about every week. In some aspects, the antibody or antigen binding portion thereof is administered at a dose of about 2 mg/kg once about every week. In some aspects, the antibody or antigen binding portion thereof is administered at a dose of about 3 mg/kg once about every week. In some aspects, the antibody or antigen binding portion thereof is administered at a dose of about 4 mg/kg once about every week. In some aspects, the antibody or antigen binding portion thereof is administered at a dose of about 5 mg/kg once about every week. In some aspects, the antibody or antigen binding portion thereof is administered at a dose of about 6 mg/kg once about every week. In some aspects, the antibody or antigen binding portion thereof is administered at a dose of about 7 mg/kg once about every week. In some aspects, the antibody or antigen binding portion thereof is administered at a dose of about 8 mg/kg once about every week. In some aspects, the antibody or antigen binding portion thereof is administered at a dose of about 9 mg/kg once about every week. In some aspects, the antibody or antigen binding portion thereof is administered at a dose of about 10 mg/kg once about every week. In some aspects, the antibody or antigen binding portion thereof is administered at a dose of about 11 mg/kg once about every week. In some aspects, the antibody or antigen binding portion thereof is administered at a dose of about 12 mg/kg once about every week. In some aspects, the antibody or antigen binding portion thereof is administered at a dose of about 13 mg/kg once about every week. In some aspects, the antibody or antigen binding portion thereof is administered at a dose of about 14 mg/kg once about every week. In some aspects, the antibody or antigen binding portion thereof is administered at a dose of about 15 mg/kg once about every week. In some aspects, the antibody or antigen binding portion thereof is administered at a dose of about 16 mg/kg once about every week. In some aspects, the antibody or antigen binding portion thereof is administered at a dose of about 17 mg/kg once about every week. In some aspects, the antibody or antigen binding portion thereof is administered at a dose of about 18 mg/kg once about every week. In some aspects, the antibody or antigen binding portion thereof is administered at a dose of about 19 mg/kg once about every week. In some aspects, the antibody or antigen binding portion thereof is administered at a dose of about 20 mg/kg once about every week.

In some aspects, the antibody or antigen binding portion thereof is administered at a dose of about 0.3 mg/kg once about every two weeks. In some aspects, the antibody or antigen binding portion thereof is administered at a dose of about 1 mg/kg once about every two weeks. In some aspects, the antibody or antigen binding portion thereof is administered at a dose of about 2 mg/kg once about every two weeks. In some aspects, the antibody or antigen binding portion thereof is administered at a dose of about 3 mg/kg once about every two weeks. In some aspects, the antibody or antigen binding portion thereof is administered at a dose of about 4 mg/kg once about every two weeks. In some aspects, the antibody or antigen binding portion thereof is administered at a dose of about 5 mg/kg once about every two weeks. In some aspects, the antibody or antigen binding portion thereof is administered at a dose of about 6 mg/kg once about every two weeks. In some aspects, the antibody or antigen binding portion thereof is administered at a dose of about 7 mg/kg once about every two weeks. In some aspects, the antibody or antigen binding portion thereof is administered at a dose of about 8 mg/kg once about every two weeks. In some aspects, the antibody or antigen binding portion thereof is administered at a dose of about 9 mg/kg once about every two weeks. In some aspects, the antibody or antigen binding portion thereof is administered at a dose of about 10 mg/kg once about every two weeks. In some aspects, the antibody or antigen binding portion thereof is administered at a dose of about 11 mg/kg once about every two weeks. In some aspects, the antibody or antigen binding portion thereof is administered at a dose of about 12 mg/kg once about every two weeks. In some aspects, the antibody or antigen binding portion thereof is administered at a dose of about 13 mg/kg once about every two weeks. In some aspects, the antibody or antigen binding portion thereof is administered at a dose of about 14 mg/kg once about every two weeks. In some aspects, the antibody or antigen binding portion thereof is administered at a dose of about 15 mg/kg once about every two weeks. In some aspects, the antibody or antigen binding portion thereof is administered at a dose of about 16 mg/kg once about every two weeks. In some aspects, the antibody or antigen binding portion thereof is administered at a dose of about 17 mg/kg once about every two weeks. In some aspects, the antibody or antigen binding portion thereof is administered at a dose of about 18 mg/kg once about every two weeks. In some aspects, the antibody or antigen binding portion thereof is administered at a dose of about 19 mg/kg once about every two weeks. In some aspects, the antibody or antigen binding portion thereof is administered at a dose of about 20 mg/kg once about every two weeks.

In some aspects, the antibody or antigen binding portion thereof is administered at a dose of about 0.3 mg/kg once about every three weeks. In some aspects, the antibody or antigen binding portion thereof is administered at a dose of about 1 mg/kg once about every three weeks. In some aspects, the antibody or antigen binding portion thereof is administered at a dose of about 2 mg/kg once about every three weeks. In some aspects, the antibody or antigen binding portion thereof is administered at a dose of about 3 mg/kg once about every three weeks. In some aspects, the antibody or antigen binding portion thereof is administered at a dose of about 4 mg/kg once about every three weeks. In some aspects, the antibody or antigen binding portion thereof is administered at a dose of about 5 mg/kg once about every three weeks. In some aspects, the antibody or antigen binding portion thereof is administered at a dose of about 6 mg/kg once about every three weeks. In some aspects, the antibody or antigen binding portion thereof is administered at a dose of about 7 mg/kg once about every three weeks. In some aspects, the antibody or antigen binding portion thereof is administered at a dose of about 8 mg/kg once about every three weeks. In some aspects, the antibody or antigen binding portion thereof is administered at a dose of about 9 mg/kg once about every three weeks. In some aspects, the antibody or antigen binding portion thereof is administered at a dose of about 10 mg/kg once about every three weeks. In some aspects, the antibody or antigen binding portion thereof is administered at a dose of about 11 mg/kg once about every three weeks. In some aspects, the antibody or antigen binding portion thereof is administered at a dose of about 12 mg/kg once about every three weeks. In some aspects, the antibody or antigen binding portion thereof is administered at a dose of about 13 mg/kg once about every three weeks. In some aspects, the antibody or antigen binding portion thereof is administered at a dose of about 14 mg/kg once about every three weeks. In some aspects, the antibody or antigen binding portion thereof is administered at a dose of about 15 mg/kg once about every three weeks. In some aspects, the antibody or antigen binding portion thereof is administered at a dose of about 16 mg/kg once about every three weeks. In some aspects, the antibody or antigen binding portion thereof is administered at a dose of about 17 mg/kg once about every three weeks. In some aspects, the antibody or antigen binding portion thereof is administered at a dose of about 18 mg/kg once about every three weeks. In some aspects, the antibody or antigen binding portion thereof is administered at a dose of about 19 mg/kg once about every three weeks. In some aspects, the antibody or antigen binding portion thereof is administered at a dose of about 20 mg/kg once about every three weeks.

In some aspects, the antibody or antigen binding portion thereof is administered at a dose of about 0.3 mg/kg once about every four weeks. In some aspects, the antibody or antigen binding portion thereof is administered at a dose of about 1 mg/kg once about every four weeks. In some aspects, the antibody or antigen binding portion thereof is administered at a dose of about 2 mg/kg once about every four weeks. In some aspects, the antibody or antigen binding portion thereof is administered at a dose of about 3 mg/kg once about every four weeks. In some aspects, the antibody or antigen binding portion thereof is administered at a dose of about 4 mg/kg once about every four weeks. In some aspects, the antibody or antigen binding portion thereof is administered at a dose of about 5 mg/kg once about every four weeks. In some aspects, the antibody or antigen binding portion thereof is administered at a dose of about 6 mg/kg once about every four weeks. In some aspects, the antibody or antigen binding portion thereof is administered at a dose of about 7 mg/kg once about every four weeks. In some aspects, the antibody or antigen binding portion thereof is administered at a dose of about 8 mg/kg once about every four weeks. In some aspects, the antibody or antigen binding portion thereof is administered at a dose of about 9 mg/kg once about every four weeks. In some aspects, the antibody or antigen binding portion thereof is administered at a dose of about 10 mg/kg once about every four weeks. In some aspects, the antibody or antigen binding portion thereof is administered at a dose of about 11 mg/kg once about every four weeks. In some aspects, the antibody or antigen binding portion thereof is administered at a dose of about 12 mg/kg once about every four weeks. In some aspects, the antibody or antigen binding portion thereof is administered at a dose of about 13 mg/kg once about every four weeks. In some aspects, the antibody or antigen binding portion thereof is administered at a dose of about 14 mg/kg once about every four weeks. In some aspects, the antibody or antigen binding portion thereof is administered at a dose of about 15 mg/kg once about every four weeks. In some aspects, the antibody or antigen binding portion thereof is administered at a dose of about 16 mg/kg once about every four weeks. In some aspects, the antibody or antigen binding portion thereof is administered at a dose of about 17 mg/kg once about every four weeks. In some aspects, the antibody or antigen binding portion thereof is administered at a dose of about 18 mg/kg once about every four weeks. In some aspects, the antibody or antigen binding portion thereof is administered at a dose of about 19 mg/kg once about every four weeks. In some aspects, the antibody or antigen binding portion thereof is administered at a dose of about 20 mg/kg once about every four weeks.

Certain aspects of the present disclosure are directed to methods of treating a cancer in a subject in need thereof. In some aspects, the cancer is selected from Kaposi's sarcoma, leukemia, acute lymphocytic leukemia, acute myelocytic leukemia, myeloblasts promyelocyte myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma (RCC), hepatocellular carcinoma (HCC), hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, retinoblastoma, rhabdomyosarcoma, rectal cancer; cancer of the respiratory system, sarcoma, skin cancer, stomach cancer, testicular cancer, thyroid cancer, uterine cancer, cancer of the urinary system, and any combination thereof. In some aspects, the cancer is chosen from lung cancer (e.g., non-small cell lung cancer), sarcoma, testicular cancer, ovarian cancer, pancreas cancer, breast cancer (e.g., triple-negative breast cancer), melanoma, head and neck cancer (e.g., squamous head and neck cancer), colorectal cancer, bladder cancer, endometrial cancer, prostate cancer, thyroid cancer, hepatocellular carcinoma, gastric cancer, brain cancer, lymphoma (e.g., DL-BCL), leukemia (e.g., AML) or renal cancer (e.g., renal cell carcinoma, e.g., clear cell RCC and/or non-clear cell RCC). In some aspects, the methods can be performed in conjunction with other therapies for cancer. For example, the composition can be administered to a subject at the same time, prior to, or after, radiation, surgery, targeted or cytotoxic chemotherapy, chemoradiotherapy, hormone therapy, immunotherapy, gene therapy, cell transplant therapy, precision medicine, genome editing therapy, or other pharmacotherapy.

In some aspects, the compositions disclosed herein are administered to a subject, e.g., a human subject, using a variety of methods that depend, in part, on the route of administration. The route can be, e.g., intravenous injection or infusion (IV), subcutaneous injection (SC), intraperitoneal (IP) injection, intramuscular injection (IM), or intrathecal injection (IT). The injection can be in a bolus or a continuous infusion.

Administration can be achieved by, e.g., local infusion, injection, or by means of an implant. The implant can be of a porous, non-porous, or gelatinous material, including membranes, such as silastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; EP488401; and EP 430539, the disclosures of each of which are incorporated herein by reference in their entirety. The composition can be delivered to the subject by way of an implantable device based on, e.g., diffusive, erodible, or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems.

In some aspects, an anti-IL-27 antibody or antigen-binding fragment thereof is therapeutically delivered to a subject by way of local administration.

In certain aspects, the route of administration is in accord with known methods, e.g. orally, through injection by intravenous, intraperitoneal, intracerebral (intra-parenchymal), intracerebroventricular, intramuscular, subcutaneously, intra-ocular, intraarterial, intraportal, or intralesional routes; by sustained release systems or by implantation devices. In certain aspects, the compositions can be administered by bolus injection or continuously by infusion, or by implantation device. In certain aspects, individual elements of the combination therapy may be administered by different routes.

In certain aspects, the composition can be administered locally via implantation of a membrane, sponge or another appropriate material onto which the desired molecule has been absorbed or encapsulated. In certain aspects, where an implantation device is used, the device can be implanted into any suitable tissue or organ, and delivery of the desired molecule can be via diffusion, timed-release bolus, or continuous administration. In certain aspects, it can be desirable to use a pharmaceutical composition comprising an anti-IL-27 antibody in an ex vivo manner. In such instances, cells, tissues and/or organs that have been removed from the patient are exposed to a pharmaceutical composition comprising an anti-IL-27 antibody after which the cells, tissues and/or organs are subsequently implanted back into the patient.

In certain aspects, an anti-IL-27 antibody can be delivered by implanting certain cells that have been genetically engineered, using methods such as those described herein, to express and secrete the polypeptides. In certain aspects, such cells can be animal or human cells, and can be autologous, heterologous, or xenogeneic. In certain aspects, the cells can be immortalized. In certain aspects, in order to decrease the chance of an immunological response, the cells can be encapsulated to avoid infiltration of surrounding tissues. In certain aspects, the encapsulation materials are typically biocompatible, semi-permeable polymeric enclosures or membranes that allow the release of the protein product(s) but prevent the destruction of the cells by the patient's immune system or by other detrimental factors from the surrounding tissues.

In some aspects, following administration antibody or antigen binding portion thereof, the subject exhibits increased expression of one or more biomarkers selected from the group consisting of Eotaxin-1 (CCL11), TARC (CCL17), VEGF-A, IL-7, IL-8, MCP-1, MCP-4, and any combination thereof; wherein the increased expression of the one or more biomarkers is relative to the expression of the one or more biomarker prior to the administration. In some aspects, following administration antibody or antigen binding portion thereof, the subject exhibits increased expression of Eotaxin-1 (CCL11), wherein the increased expression of Eotaxin-1 (CCL11) is relative to the expression of Eotaxin-1 (CCL11) prior to the administration.

In some aspects, following administration antibody or antigen binding portion thereof, the subject exhibits increased expression of TARC (CCL17), wherein the increased expression of TARC (CCL17) is relative to the expression of TARC (CCL17) prior to the administration.

In some aspects, following administration antibody or antigen binding portion thereof, the subject exhibits increased expression of VEGF-A, wherein the increased expression of VEGF-A is relative to the expression of VEGF-A prior to the administration.

In some aspects, following administration antibody or antigen binding portion thereof, the subject exhibits increased expression of IL-7, wherein the increased expression of IL-7 is relative to the expression of IL-7 prior to the administration.

In some aspects, following administration antibody or antigen binding portion thereof, the subject exhibits increased expression of IL-8, wherein the increased expression of IL-8 is relative to the expression of IL-8 prior to the administration.

In some aspects, following administration antibody or antigen binding portion thereof, the subject exhibits increased expression of MCP-1, wherein the increased expression of MCP-1 is relative to the expression of MCP-1 prior to the administration.

In some aspects, following administration antibody or antigen binding portion thereof, the subject exhibits increased expression of MCP-4, wherein the increased expression of MCP-4 is relative to the expression of MCP-4 prior to the administration.

### C. IL-27 Inhibiting Agents

Some aspects of the present disclosure are directed to methods of administering an IL-27 inhibiting agent to a subject, wherein the subject is identified as being suitable for an IL-27-inhibiting agent therapy. Any molecule that is capable of inhibiting the activity of IL-27 or its receptor (WSX-1) can be used in the methods disclosed herein. In some aspects, the IL-27 inhibiting agent reduces or blocks the interaction between IL-27 and WSX-1. In some aspects, the inhibitor reduces or blocks downstream IL-27 signaling.

In some aspects, the IL-27 inhibiting agent comprises a polypeptide. In some aspects, the IL-27 inhibiting agent comprises a small molecule. In some aspects, the IL-27 inhibiting agent comprises an antibody or an antigen-binding portion thereof that specifically binds to human IL-27 (“anti-IL-27 antibody”). In some aspects, the antibody or an antigen-binding portion thereof specifically binds to IL-27p28 and antagonize IL-27, in particular human IL-27.

In some aspects, the antibody or antigen binding portion thereof inhibits or reduces STAT1 and/or STAT3 phosphorylation in a cell in the subject. In some aspects, the antibody or antigen binding portion thereof inhibits or reduces pSTAT1 signaling (e.g., IL-27 mediated pSTAT1 signaling). In some aspects, the antibody or antigen binding portion thereof inhibits or reduces inhibition of CD161 expression in a cell in the subject. In some aspects, the antibody or antigen binding portion thereof inhibits or reduces PD-L1 expression in a cell in the subject. In some aspects, the antibody or antigen binding portion thereof induces or enhances PD-1 mediated secretion of one or more cytokines from a cell in the subject. In some aspects, the antibody or antigen binding portion thereof alters TIM-3 expression in a cell in the subject. In some aspects, the cell is a tumor cell or an immune cell.

In some aspects, the antibody or antigen binding portion thereof specifically binds to an epitope comprising one or more amino acids of (i) amino acids 37 to 56 corresponding to SEQ ID NO: 2 (IL-27p28), (ii) amino acids 142 to 164 corresponding to SEQ ID NO: 2 (IL-27p28), or (iii) both (i) and (ii). In some aspects, an isolated antibody of the disclosure that antagonizes human IL-27, or an antigen binding portion thereof, specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, or Glu164 of SEQ ID NO: 2 (IL-27p28).

In some aspects, the antibody or the antigen binding portion thereof comprises a heavy chain CDR3 comprising the sequence set forth in SEQ ID NO: 121. In some aspects, the antibody or the antigen binding portion thereof comprises a heavy chain CDR3 comprising the sequence set forth in SEQ ID NO: 124. In some aspects, the antibody or the antigen binding portion thereof comprises a heavy chain CDR2 comprising the sequence set forth in SEQ ID NO: 120 or 123. In some aspects, the antibody or the antigen binding portion thereof comprises a heavy chain CDR2 comprising the sequence set forth in SEQ ID NO: 120. In some aspects, the antibody or the antigen binding portion thereof comprises a heavy chain CDR2 comprising the sequence set forth in SEQ ID NO: 123. In some aspects, the antibody or the antigen binding portion thereof comprises a heavy chain CDR1 comprising the sequence set forth in SEQ ID NO: 119 or 122. In some aspects, the antibody or the antigen binding portion thereof comprises a heavy chain CDR1 comprising the sequence set forth in SEQ ID NO: 119. In some aspects, the antibody or the antigen binding portion thereof comprises a heavy chain CDR1 comprising the sequence set forth in SEQ ID NO: 122. In some aspects, the antibody or the antigen binding portion thereof comprises a light chain CDR3 comprising the sequence set forth in SEQ ID NO: 129 or 132. In some aspects, the antibody or the antigen binding portion thereof comprises a light chain CDR3 comprising the sequence set forth in SEQ ID NO: 129. In some aspects, the antibody or the antigen binding portion thereof comprises a light chain CDR3 comprising the sequence set forth in SEQ ID NO: 132. In some aspects, the antibody or the antigen binding portion thereof comprises a light chain CDR2 comprising the sequence set forth in SEQ ID NO: 128 or 131. In some aspects, the antibody or the antigen binding portion thereof comprises a light chain CDR2 comprising the sequence set forth in SEQ ID NO: 128. In some aspects, the antibody or the antigen binding portion thereof comprises a light chain CDR2 comprising the sequence set forth in SEQ ID NO: 131. In some aspects, the antibody or the antigen binding portion thereof comprises a light chain CDR1 comprising the sequence set forth in SEQ ID NO: 127 or 130. In some aspects, the antibody or the antigen binding portion thereof comprises a light chain CDR1 comprising the sequence set forth in SEQ ID NO: 127. In some aspects, the antibody or the antigen binding portion thereof comprises a light chain CDR1 comprising the sequence set forth in SEQ ID NO: 130.

In some aspects, the antibody or the antigen binding portion thereof comprises: a heavy chain CDR1 comprising the amino acid sequence set forth in SEQ ID NO: 119, a heavy chain CDR2 comprising the amino acid sequence set forth in SEQ ID NO: 120, and a heavy chain CDR3 comprising the amino acid sequence set forth in SEQ ID NO: 121. In some aspects, the antibody or the antigen binding portion thereof comprises: a heavy chain CDR1 comprising the amino acid sequence set forth in SEQ ID NO: 122, a heavy chain CDR2 comprising the amino acid sequence set forth in SEQ ID NO: 123, and a heavy chain CDR3 comprising the amino acid sequence set forth in SEQ ID NO: 124.

In some aspects, the antibody or the antigen binding portion thereof comprises: a light chain CDR1 comprising the amino acid sequence set forth in SEQ ID NO: 127, a light chain CDR2 comprising the amino acid sequence set forth in SEQ ID NO: 128, and a light chain CDR3 comprising the amino acid sequence set forth in SEQ ID NO: 129. In some aspects, the antibody or the antigen binding portion thereof comprises: a light chain CDR1 comprising the amino acid sequence set forth in SEQ ID NO: 130, a light chain CDR2 comprising the amino acid sequence set forth in SEQ ID NO: 131, and a light chain CDR3 comprising the amino acid sequence set forth in SEQ ID NO: 132.

In some aspects, the antibody or the antigen binding portion thereof comprises: a heavy chain CDR1 comprising the amino acid sequence set forth in SEQ ID NO: 119, a heavy chain CDR2 comprising the amino acid sequence set forth in SEQ ID NO: 120, a heavy chain CDR3 comprising the amino acid sequence set forth in SEQ ID NO: 121, a light chain CDR1 comprising the amino acid sequence set forth in SEQ ID NO: 127, a light chain CDR2 comprising the amino acid sequence set forth in SEQ ID NO: 128, and a light chain CDR3 comprising the amino acid sequence set forth in SEQ ID NO: 129.

In some aspects, the antibody or the antigen binding portion thereof comprises a heavy chain variable region comprising an amino acid sequence that has at least about 85%, at least about 90%, at least about 95%, at least about 96%, at least about 97%, at least about 98%, or at least about 99% sequence identity to the amino acid sequence set forth in SEQ ID NO: 125. In some aspects, the antibody or the antigen binding portion thereof comprises a heavy chain variable region comprising the amino acid sequence set forth in SEQ ID NO: 125.

In some aspects, the antibody or the antigen binding portion thereof comprises a light chain variable region comprising an amino acid sequence that has at least about 85%, at least about 90%, at least about 95%, at least about 96%, at least about 97%, at least about 98%, or at least about 99% sequence identity to the amino acid sequence set forth in SEQ ID NO: 133. In some aspects, the antibody or the antigen binding portion thereof comprises a light chain variable region comprising the amino acid sequence set forth in SEQ ID NO: 133.

In some aspects, the antibody or the antigen binding portion thereof comprises a heavy chain comprising an amino acid sequence that has at least about 85%, at least about 90%, at least about 95%, at least about 96%, at least about 97%, at least about 98%, or at least about 99% sequence identity to the amino acid sequence set forth in SEQ ID NO: 135. In some aspects, the antibody or the antigen binding portion thereof comprises a heavy chain comprising the amino acid sequence set forth in SEQ ID NO: 135.

In some aspects, the antibody or the antigen binding portion thereof comprises a heavy chain comprising an amino acid sequence that has at least about 85%, at least about 90%, at least about 95%, at least about 96%, at least about 97%, at least about 98%, or at least about 99% sequence identity to the amino acid sequence set forth in SEQ ID NO: 139. In some aspects, the antibody or the antigen binding portion thereof comprises a heavy chain comprising the amino acid sequence set forth in SEQ ID NO: 139.

In some aspects, the antibody or the antigen binding portion thereof comprises a light chain comprising an amino acid sequence that has at least about 85%, at least about 90%, at least about 95%, at least about 96%, at least about 97%, at least about 98%, or at least about 99% sequence identity to the amino acid sequence set forth in SEQ ID NO: 137. In some aspects, the antibody or the antigen binding portion thereof comprises a light chain comprising the amino acid sequence set forth in SEQ ID NO: 137.

In some aspects, the anti-IL-27 antibody is administered at a dose of at least about 0.003 mg/kg to at least about 20 mg/kg. In some aspects, the anti-IL-27 antibody is administered at a dose of at least about 1 mg/kg. In some aspects, the anti-IL-27 antibody is administered at a dose of at least about 2 mg/kg. In some aspects, the anti-IL-27 antibody is administered at a dose of at least about 3 mg/kg. In some aspects, the anti-IL-27 antibody is administered at a dose of at least about 4 mg/kg. In some aspects, the anti-IL-27 antibody is administered at a dose of at least about 5 mg/kg. In some aspects, the anti-IL-27 antibody is administered at a dose of at least about 6 mg/kg. In some aspects, the anti-IL-27 antibody is administered at a dose of at least about 7 mg/kg. In some aspects, the anti-IL-27 antibody is administered at a dose of at least about 8 mg/kg. In some aspects, the anti-IL-27 antibody is administered at a dose of at least about 9 mg/kg. In some aspects, the anti-IL-27 antibody is administered at a dose of at least about 10 mg/kg. In some aspects, the anti-IL-27 antibody is administered at a dose of at least about 11 mg/kg. In some aspects, the anti-IL-27 antibody is administered at a dose of at least about 12 mg/kg. In some aspects, the anti-IL-27 antibody is administered at a dose of at least about 13 mg/kg. In some aspects, the anti-IL-27 antibody is administered at a dose of at least about 14 mg/kg. In some aspects, the anti-IL-27 antibody is administered at a dose of at least about 15 mg/kg. In some aspects, the anti-IL-27 antibody is administered at a dose of at least about 16 mg/kg. In some aspects, the anti-IL-27 antibody is administered at a dose of at least about 17 mg/kg. In some aspects, the anti-IL-27 antibody is administered at a dose of at least about 18 mg/kg. In some aspects, the anti-IL-27 antibody is administered at a dose of at least about 19 mg/kg. In some aspects, the anti-IL-27 antibody is administered at a dose of at least about 20 mg/kg.

In some aspects, the antibody or antigen-binding portion thereof comprises an amino acid sequence set forth in Table 1A.

TABLE 1A
Anti-IL-27 Antibodies

| | | |
|---|---|---|
| SEQ | | |
| ID NO | Description | Sequence |

anti-IL-27 Ab2-A

| | | |
|---|---|---|
| 9 | HCDR1 | GFTFSSYS |
| | (IMGT) | |
| 10 | HCDR2 | ISSSSSYI |
| | (IMGT) | |
| 11 | HCDR3 | ARDGGRTSYTATAHNWFDP |
| | (IMGT) | |
| 12 | HCDR1 (NT) | FTFSSYSMN |
| 13 | HCDR2 (NT) | SISSSSSYIYYADSVKG |
| 14 | HCDR3 (NT) | ARDGGRTSYTATAHNWFDP |
| 15 | VH | EVQLVESGGGLVKPGGSLRLSCAASGFTFSSYSMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSS |
| 16 | DNA VH | GAGGTGCAGCTGGTGGAGTCTGGGGGAGGCCTGGTCAAGCCTGG |
| | | GGGGTCCCTGAGACTCTCCTGTGCAGCCTCTGGATTCACCTTCA |
| | | GTAGCTATAGCATGAACTGGGTCCGCCAGGCTCCAGGGAAGGGG |
| | | CTGGAGTGGGTCTCATCCATTAGTAGTAGTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCA |
| 17 | LCDR1 | QSVLFSSNNKNY |
| | (IMGT) | |
| 18 | LCDR2 | WAS |
| | (IMGT) | |
| 19 | LCDR3 | QQHASAPPT |
| | (IMGT) | |
| 20 | LCDR1 (NT) | KSSQSVLFSSNNKNYLA |
| 21 | LCDR2 (NT) | WASTRES |
| 22 | LCDR3 (NT) | QQHASAPPT |
| 23 | VL | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIK |
| 24 | DNA VL | GACATCGTGATGACCCAGTCTCCAGACTCCCTGGCTGTGTCTCT |
| | | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAA |
| 25 | Heavy Chain | EVQLVESGGGLVKPGGSLRLSCAASGFTFSSYSMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISR |
| | | TPEVTCVVVDVSHEDPEVKFNWYVDGVEVHNAKTKPREEQYNST |
| | | YRVVSVLTVLHQDWLNGKEYKCKVSNKALPAPIEKTISKAKGQP |
| | | REPQVYTLPPSRDELTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 26 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCA |
| | | GTAGCTATAGCATGAACTGGGTCCGCCAGGCTCCAGGGAAGGGG |
| | | CTGGAGTGGGTCTCATCCATTAGTAGTAGTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCGAGCACCAAAGGCCCG |
| | | AGCGTGTTTCCGCTGGCGCCGAGCAGCAAAAGCACCAGCGGCGG |
| | | CACCGCGGCGCTGGGCTGCCTGGTGAAAGATTATTTTCCGGAAC |
| | | CGGTGACCGTGAGCTGGAACAGCGGCGCGCTGACCAGCGGCGTG |
| | | CATACCTTTCCGGCGGTGCTGCAGAGCAGCGGCCTGTATAGCCT |
| | | GAGCAGCGTGGTGACCGTGCCGAGCAGCAGCCTGGGCACCCAGA |
| | | CCTATATTTGCAACGTGAACCATAAACCGAGCAACACCAAAGTG |
| | | GATAAAAAAGTGGAACCGAAAAGCTGCGATAAAACCCATACCTG |
| | | CCCGCCGTGCCCGGCGCCGGAACTGCTGGGCGGCCCGAGCGTGT |
| | | TTCTGTTTCCGCCGAAACCGAAAGATACCCTGATGATTAGCCGC |
| | | ACCCCGGAAGTGACCTGCGTGGTGGTGGATGTGAGCCATGAAGA |
| | | TCCGGAAGTGAAATTTAACTGGTATGTGGATGGCGTGGAAGTGC |
| | | ATAACGCGAAAACCAAACCGCGCGAAGAACAGTATAACAGCACC |
| | | TATCGCGTGGTGAGCGTGCTGACCGTGCTGCATCAGGATTGGCT |
| | | GAACGGCAAAGAATATAAATGCAAAGTGAGCAACAAAGCGCTGC |
| | | CGGCGCCGATTGAAAAAACCATTAGCAAAGCGAAAGGCCAGCCG |
| | | CGCGAACCGCAGGTGTATACCCTGCCGCCGAGCCGCGATGAACT |
| | | GACCAAAAACCAGGTGAGCCTGACCTGCCTGGTGAAAGGCTTTT |
| | | ATCCGAGCGATATTGCGGTGGAATGGGAAAGCAACGGCCAGCCG |
| | | GAAAACAACTATAAAACCACCCCGCCGGTGCTGGATAGCGATGG |
| | | CAGCTTTTTTCTGTATAGCAAACTGACCGTGGATAAAAGCCGCT |
| | | GGCAGCAGGGCAACGTGTTTAGCTGCAGCGTGATGCATGAAGCG |
| | | CTGCATAACCATTATACCCAGAAAAGCCTGAGCCTGAGCCCGGG |
| | | CAAA |
| 27 | Light Chain | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIKRTVAAPSVFIFPPSDEQLK |
| | | SGTASVVCLLNNFYPREAKVQWKVDNALQSGNSQESVTEQDSKD |
| | | STYSLSSTLTLSKADYEKHKVYACEVTHQGLSSPVTKSFNRGEC |
| 28 | DNA Light | GACATCGTGATGACCCAGTCTCCAGACTCCCTGGCTGTGTCTCT |
| | Chain | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAACGTACGGTGGCCG |
| | | CTCCCTCCGTGTTCATCTTCCCACCCTCCGACGAGCAGCTGAAG |
| | | TCCGGCACCGCCTCCGTCGTGTGCCTGCTGAACAACTTCTACCC |
| | | TCGCGAGGCCAAAGTGCAGTGGAAAGTGGACAACGCCCTGCAGT |
| | | CCGGCAACTCCCAGGAATCCGTCACCGAGCAGGACTCCAAGGAC |
| | | AGCACCTACTCCCTGTCCTCCACCCTGACCCTGTCCAAGGCCGA |
| | | CTACGAGAAGCACAAAGTGTACGCCTGCGAAGTGACCCACCAGG |
| | | GCCTGTCCAGCCCCGTGACCAAGTCCTTCAACCGGGGCGAGTGC |

anti-IL-27 Ab2-B

| | | |
|---|---|---|
| 29 | Heavy Chain | EVQLVESGGGLVKPGGSLRLSCAASGFTFSSYSMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSSASTKGP |
| | | SVFPLAPCSRSTSESTAALGCLVKDYFPEPVTVSWNSGALTSGV |
| | | HTFPAVLQSSGLYSLSSVVTVPSSSLGTKTYTCNVDHKPSNTKV |
| | | DKRVESKYGPPCPSCPAPEFLGGPSVFLFPPKPKDTLMISRTPE |
| | | VTCVVVDVSQEDPEVQFNWYVDGVEVHNAKTKPREEQFNSTYRV |
| | | VSVLTVLHQDWLNGKEYKCKVSNKGLPSSIEKTISKAKGQPREP |
| | | QVYTLPPSQEEMTKNQVSLTCLVKGFYPSDIAVEWESNGQPENN |
| | | YKTTPPVLDSDGSFFLYSRLTVDKSRWQEGNVFSCSVMHEALHN |
| | | HYTQKSLSLSLG |
| 30 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCA |
| | | GTAGCTATAGCATGAACTGGGTCCGCCAGGCTCCAGGGAAGGGG |
| | | CTGGAGTGGGTCTCATCCATTAGTAGTAGTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCTTCCACCAAGGGCCCC |
| | | TCCGTGTTCCCTCTGGCCCCTTGCTCCCGGTCCACCTCCGAGTC |
| | | TACCGCCGCTCTGGGCTGCCTCGTGAAGGACTACTTCCCCGAGC |
| | | CCGTGACCGTGTCCTGGAACTCTGGCGCCCTGACCTCCGGCGTG |
| | | CACACCTTCCCTGCCGTGCTGCAGTCCTCCGGCCTGTACTCCCT |
| | | GTCCAGCGTCGTGACCGTGCCCTCCTCCAGCCTGGGCACCAAGA |
| | | CCTACACCTGTAACGTGGACCACAAGCCCTCCAACACCAAAGTG |
| | | GACAAGCGGGTGGAATCTAAGTACGGCCCTCCCTGCCCTTCCTG |
| | | CCCTGCCCCTGAGTTCCTGGGCGGACCTTCCGTGTTCCTGTTCC |
| | | CTCCAAAGCCCAAGGACACCCTGATGATCTCCCGGACCCCTGAA |
| | | GTGACCTGCGTGGTGGTGGACGTGTCCCAGGAAGATCCCGAAGT |
| | | CCAGTTCAATTGGTACGTGGACGGCGTGGAAGTGCACAACGCCA |
| | | AGACCAAGCCCAGAGAGGAACAGTTCAACTCCACCTACCGGGTG |
| | | GTGTCCGTGCTGACCGTGCTGCACCAGGACTGGCTGAACGGCAA |
| | | AGAGTACAAGTGCAAAGTGTCCAACAAGGGCCTGCCCTCCAGCA |
| | | TCGAAAAGACCATCTCCAAGGCCAAGGGCCAGCCCCGCGAGCCC |
| | | CAAGTGTACACCCTGCCTCCCAGCCAGGAAGAGATGACCAAGAA |
| | | TCAAGTGTCCCTGACTTGTCTGGTCAAGGGCTTCTACCCCTCCG |
| | | ATATCGCCGTGGAGTGGGAGTCCAACGGCCAGCCCGAGAACAAC |
| | | TACAAGACCACCCCTCCCGTGCTGGACTCCGACGGCTCCTTCTT |
| | | CCTGTACTCTCGGCTGACCGTGGACAAGTCCCGGTGGCAGGAAG |
| | | GCAACGTCTTCTCCTGCTCCGTGATGCACGAGGCCCTGCACAAC |
| | | CACTACACCCAGAAGTCCCTGTCCCTGTCTCTGGGC |

anti-IL-27 Ab3-A

| | | |
|---|---|---|
| 31 | HCDR1 | GFTFRSYG |
| | (IMGT) | |
| 32 | HCDR2 | ISSSSSYI |
| | (IMGT) | |
| 33 | HCDR3 | ARDGGRTSYTATAHNWFDP |
| | (IMGT) | |
| 34 | HCDR1 (NT) | FTFRSYGMN |
| 35 | HCDR2 (NT) | SISSSSSYIYYADSVKG |
| 36 | HCDR3 (NT) | ARDGGRTSYTATAHNWFDP |
| 37 | VH | EVQLVESGGGLVKPGGSLRLSCAASGFTFRSYGMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSS |
| 38 | DNA VH | GAGGTGCAGCTGGTGGAGTCTGGGGGAGGCCTGGTCAAGCCTGG |
| | | GGGGTCCCTGAGACTCTCCTGTGCAGCCTCTGGATTCACCTTCC |
| | | GGAGCTATGGGATGAACTGGGTCCGCCAGGCTCCAGGGAAGGGG |
| | | CTGGAGTGGGTCTCATCCATTAGTAGTAGTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCA |
| 39 | LCDR1 | QSVLFSSNNKNY |
| | (IMGT) | |
| 40 | LCDR2 | WAS |
| | (IMGT) | |
| 41 | LCDR3 | QQHASAPPT |
| | (IMGT) | |
| 42 | LCDR1 (NT) | KSSQSVLFSSNNKNYLA |
| 43 | LCDR2 (NT) | WASTRES |
| 44 | LCDR3 (NT) | QQHASAPPT |
| 45 | VL | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIK |
| 46 | DNA VL | GACATCGTGATGACCCAGTCTCCAGACTCCCTGGCTGTGTCTCT |
| | | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAA |
| 47 | Heavy Chain | EVQLVESGGGLVKPGGSLRLSCAASGFTFRSYGMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISR |
| | | TPEVTCVVVDVSHEDPEVKFNWYVDGVEVHNAKTKPREEQYNST |
| | | YRVVSVLTVLHQDWLNGKEYKCKVSNKALPAPIEKTISKAKGQP |
| | | REPQVYTLPPSRDELTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 48 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCC |
| | | GGAGCTATGGGATGAACTGGGTCCGCCAGGCTCCAGGGAAGGGG |
| | | CTGGAGTGGGTCTCATCCATTAGTAGTAGTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCGAGCACCAAAGGCCCG |
| | | AGCGTGTTTCCGCTGGCGCCGAGCAGCAAAAGCACCAGCGGCGG |
| | | CACCGCGGCGCTGGGCTGCCTGGTGAAAGATTATTTTCCGGAAC |
| | | CGGTGACCGTGAGCTGGAACAGCGGCGCGCTGACCAGCGGCGTG |
| | | CATACCTTTCCGGCGGTGCTGCAGAGCAGCGGCCTGTATAGCCT |
| | | GAGCAGCGTGGTGACCGTGCCGAGCAGCAGCCTGGGCACCCAGA |
| | | CCTATATTTGCAACGTGAACCATAAACCGAGCAACACCAAAGTG |
| | | GATAAAAAAGTGGAACCGAAAAGCTGCGATAAAACCCATACCTG |
| | | CCCGCCGTGCCCGGCGCCGGAACTGCTGGGCGGCCCGAGCGTGT |
| | | TTCTGTTTCCGCCGAAACCGAAAGATACCCTGATGATTAGCCGC |
| | | ACCCCGGAAGTGACCTGCGTGGTGGTGGATGTGAGCCATGAAGA |
| | | TCCGGAAGTGAAATTTAACTGGTATGTGGATGGCGTGGAAGTGC |
| | | ATAACGCGAAAACCAAACCGCGCGAAGAACAGTATAACAGCACC |
| | | TATCGCGTGGTGAGCGTGCTGACCGTGCTGCATCAGGATTGGCT |
| | | GAACGGCAAAGAATATAAATGCAAAGTGAGCAACAAAGCGCTGC |
| | | CGGCGCCGATTGAAAAAACCATTAGCAAAGCGAAAGGCCAGCCG |
| | | CGCGAACCGCAGGTGTATACCCTGCCGCCGAGCCGCGATGAACT |
| | | GACCAAAAACCAGGTGAGCCTGACCTGCCTGGTGAAAGGCTTTT |
| | | ATCCGAGCGATATTGCGGTGGAATGGGAAAGCAACGGCCAGCCG |
| | | GAAAACAACTATAAAACCACCCCGCCGGTGCTGGATAGCGATGG |
| | | CAGCTTTTTTCTGTATAGCAAACTGACCGTGGATAAAAGCCGCT |
| | | GGCAGCAGGGCAACGTGTTTAGCTGCAGCGTGATGCATGAAGCG |
| | | CTGCATAACCATTATACCCAGAAAAGCCTGAGCCTGAGCCCGGG |
| | | CAAA |
| 49 | Light Chain | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIKRTVAAPSVFIFPPSDEQLK |
| | | SGTASVVCLLNNFYPREAKVQWKVDNALQSGNSQESVTEQDSKD |
| | | STYSLSSTLTLSKADYEKHKVYACEVTHQGLSSPVTKSFNRGEC |
| 50 | DNA Light | GACATCGTGATGACCCAGTCTCCAGACTCCCTGGCTGTGTCTCT |
| | Chain | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAACGTACGGTGGCCG |
| | | CTCCCTCCGTGTTCATCTTCCCACCCTCCGACGAGCAGCTGAAG |
| | | TCCGGCACCGCCTCCGTCGTGTGCCTGCTGAACAACTTCTACCC |
| | | TCGCGAGGCCAAAGTGCAGTGGAAAGTGGACAACGCCCTGCAGT |
| | | CCGGCAACTCCCAGGAATCCGTCACCGAGCAGGACTCCAAGGAC |
| | | AGCACCTACTCCCTGTCCTCCACCCTGACCCTGTCCAAGGCCGA |
| | | CTACGAGAAGCACAAAGTGTACGCCTGCGAAGTGACCCACCAGG |
| | | GCCTGTCCAGCCCCGTGACCAAGTCCTTCAACCGGGGCGAGTGC |

anti-IL-27 Ab3-B

| | | |
|---|---|---|
| 51 | Heavy Chain | EVQLVESGGGLVKPGGSLRLSCAASGFTFRSYGMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSSASTKGP |
| | | SVFPLAPCSRSTSESTAALGCLVKDYFPEPVTVSWNSGALTSGV |
| | | HTFPAVLQSSGLYSLSSVVTVPSSSLGTKTYTCNVDHKPSNTKV |
| | | DKRVESKYGPPCPSCPAPEFLGGPSVFLFPPKPKDTLMISRTPE |
| | | VTCVVVDVSQEDPEVQFNWYVDGVEVHNAKTKPREEQFNSTYRV |
| | | VSVLTVLHQDWLNGKEYKCKVSNKGLPSSIEKTISKAKGQPREP |
| | | QVYTLPPSQEEMTKNQVSLTCLVKGFYPSDIAVEWESNGQPENN |
| | | YKTTPPVLDSDGSFFLYSRLTVDKSRWQEGNVFSCSVMHEALHN |
| | | HYTQKSLSLSLG |
| 52 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCC |
| | | GGAGCTATGGGATGAACTGGGTCCGCCAGGCTCCAGGGAAGGGG |
| | | CTGGAGTGGGTCTCATCCATTAGTAGTAGTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCTTCCACCAAGGGCCCC |
| | | TCCGTGTTCCCTCTGGCCCCTTGCTCCCGGTCCACCTCCGAGTC |
| | | TACCGCCGCTCTGGGCTGCCTCGTGAAGGACTACTTCCCCGAGC |
| | | CCGTGACCGTGTCCTGGAACTCTGGCGCCCTGACCTCCGGCGTG |
| | | CACACCTTCCCTGCCGTGCTGCAGTCCTCCGGCCTGTACTCCCT |
| | | GTCCAGCGTCGTGACCGTGCCCTCCTCCAGCCTGGGCACCAAGA |
| | | CCTACACCTGTAACGTGGACCACAAGCCCTCCAACACCAAAGTG |
| | | GACAAGCGGGTGGAATCTAAGTACGGCCCTCCCTGCCCTTCCTG |
| | | CCCTGCCCCTGAGTTCCTGGGCGGACCTTCCGTGTTCCTGTTCC |
| | | CTCCAAAGCCCAAGGACACCCTGATGATCTCCCGGACCCCTGAA |
| | | GTGACCTGCGTGGTGGTGGACGTGTCCCAGGAAGATCCCGAAGT |
| | | CCAGTTCAATTGGTACGTGGACGGCGTGGAAGTGCACAACGCCA |
| | | AGACCAAGCCCAGAGAGGAACAGTTCAACTCCACCTACCGGGTG |
| | | GTGTCCGTGCTGACCGTGCTGCACCAGGACTGGCTGAACGGCAA |
| | | AGAGTACAAGTGCAAAGTGTCCAACAAGGGCCTGCCCTCCAGCA |
| | | TCGAAAAGACCATCTCCAAGGCCAAGGGCCAGCCCCGCGAGCCC |
| | | CAAGTGTACACCCTGCCTCCCAGCCAGGAAGAGATGACCAAGAA |
| | | TCAAGTGTCCCTGACTTGTCTGGTCAAGGGCTTCTACCCCTCCG |
| | | ATATCGCCGTGGAGTGGGAGTCCAACGGCCAGCCCGAGAACAAC |
| | | TACAAGACCACCCCTCCCGTGCTGGACTCCGACGGCTCCTTCTT |
| | | CCTGTACTCTCGGCTGACCGTGGACAAGTCCCGGTGGCAGGAAG |
| | | GCAACGTCTTCTCCTGCTCCGTGATGCACGAGGCCCTGCACAAC |
| | | CACTACACCCAGAAGTCCCTGTCCCTGTCTCTGGGC |

anti-IL-27 Ab4-A

| | | |
|---|---|---|
| 53 | HCDR1 | GFTFSRTG |
| | (IMGT) | |
| 54 | HCDR2 | ISSSSSYI |
| | (IMGT) | |
| 55 | HCDR3 | ARDGGRTSYTATAHNWFDP |
| | (IMGT) | |
| 56 | HCDR1 (NT) | FTFSRTGMN |
| 57 | HCDR2 (NT) | SISSSSSYIYYADSVKG |
| 58 | HCDR3 (NT) | ARDGGRTSYTATAHNWFDP |
| 59 | VH | EVQLVESGGGLVKPGGSLRLSCAASGFTFSRTGMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSS |
| 60 | DNA VH | GAGGTGCAGCTGGTGGAGTCTGGGGGAGGCCTGGTCAAGCCTGG |
| | | GGGGTCCCTGAGACTCTCCTGTGCAGCCTCTGGATTCACCTTCA |
| | | GTAGGACTGGGATGAACTGGGTCCGCCAGGCTCCAGGGAAGGGG |
| | | CTGGAATGGGTCTCATCCATTAGTAGTAGTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCA |
| 61 | LCDR1 | QSVLFSSNNKNY |
| | (IMGT) | |
| 62 | LCDR2 | WAS |
| | (IMGT) | |
| 63 | LCDR3 | QQHASAPPT |
| | (IMGT) | |
| 64 | LCDR1 (NT) | KSSQSVLFSSNNKNYLA |
| 65 | LCDR2 (NT) | WASTRES |
| 66 | LCDR3 (NT) | QQHASAPPT |
| 67 | VL | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIK |
| 68 | DNA VL | GACATCGTGATGACCCAGTCTCCAGACTCCCTGGCTGTGTCTCT |
| | | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAA |
| 69 | Heavy Chain | EVQLVESGGGLVKPGGSLRLSCAASGFTFSRTGMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISR |
| | | TPEVTCVVVDVSHEDPEVKFNWYVDGVEVHNAKTKPREEQYNST |
| | | YRVVSVLTVLHQDWLNGKEYKCKVSNKALPAPIEKTISKAKGQP |
| | | REPQVYTLPPSRDELTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 70 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCA |
| | | GTAGGACTGGGATGAACTGGGTCCGCCAGGCTCCAGGGAAGGGG |
| | | CTGGAATGGGTCTCATCCATTAGTAGTAGTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCGAGCACCAAAGGCCCG |
| | | AGCGTGTTTCCGCTGGCGCCGAGCAGCAAAAGCACCAGCGGCGG |
| | | CACCGCGGCGCTGGGCTGCCTGGTGAAAGATTATTTTCCGGAAC |
| | | CGGTGACCGTGAGCTGGAACAGCGGCGCGCTGACCAGCGGCGTG |
| | | CATACCTTTCCGGCGGTGCTGCAGAGCAGCGGCCTGTATAGCCT |
| | | GAGCAGCGTGGTGACCGTGCCGAGCAGCAGCCTGGGCACCCAGA |
| | | CCTATATTTGCAACGTGAACCATAAACCGAGCAACACCAAAGTG |
| | | GATAAAAAAGTGGAACCGAAAAGCTGCGATAAAACCCATACCTG |
| | | CCCGCCGTGCCCGGCGCCGGAACTGCTGGGCGGCCCGAGCGTGT |
| | | TTCTGTTTCCGCCGAAACCGAAAGATACCCTGATGATTAGCCGC |
| | | ACCCCGGAAGTGACCTGCGTGGTGGTGGATGTGAGCCATGAAGA |
| | | TCCGGAAGTGAAATTTAACTGGTATGTGGATGGCGTGGAAGTGC |
| | | ATAACGCGAAAACCAAACCGCGCGAAGAACAGTATAACAGCACC |
| | | TATCGCGTGGTGAGCGTGCTGACCGTGCTGCATCAGGATTGGCT |
| | | GAACGGCAAAGAATATAAATGCAAAGTGAGCAACAAAGCGCTGC |
| | | CGGCGCCGATTGAAAAAACCATTAGCAAAGCGAAAGGCCAGCCG |
| | | CGCGAACCGCAGGTGTATACCCTGCCGCCGAGCCGCGATGAACT |
| | | GACCAAAAACCAGGTGAGCCTGACCTGCCTGGTGAAAGGCTTTT |
| | | ATCCGAGCGATATTGCGGTGGAATGGGAAAGCAACGGCCAGCCG |
| | | GAAAACAACTATAAAACCACCCCGCCGGTGCTGGATAGCGATGG |
| | | CAGCTTTTTTCTGTATAGCAAACTGACCGTGGATAAAAGCCGCT |
| | | GGCAGCAGGGCAACGTGTTTAGCTGCAGCGTGATGCATGAAGCG |
| | | CTGCATAACCATTATACCCAGAAAAGCCTGAGCCTGAGCCCGGG |
| | | CAAA |
| 71 | Light Chain | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIKRTVAAPSVFIFPPSDEQLK |
| | | SGTASVVCLLNNFYPREAKVQWKVDNALQSGNSQESVTEQDSKD |
| | | STYSLSSTLTLSKADYEKHKVYACEVTHQGLSSPVTKSFNRGEC |
| 72 | DNA Light | GACATCGTGATGACCCAGTCTCCAGACTCCCTGGCTGTGTCTCT |
| | Chain | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAACGTACGGTGGCCG |
| | | CTCCCTCCGTGTTCATCTTCCCACCCTCCGACGAGCAGCTGAAG |
| | | TCCGGCACCGCCTCCGTCGTGTGCCTGCTGAACAACTTCTACCC |
| | | TCGCGAGGCCAAAGTGCAGTGGAAAGTGGACAACGCCCTGCAGT |
| | | CCGGCAACTCCCAGGAATCCGTCACCGAGCAGGACTCCAAGGAC |
| | | AGCACCTACTCCCTGTCCTCCACCCTGACCCTGTCCAAGGCCGA |
| | | CTACGAGAAGCACAAAGTGTACGCCTGCGAAGTGACCCACCAGG |
| | | GCCTGTCCAGCCCCGTGACCAAGTCCTTCAACCGGGGCGAGTGC |

anti-IL-27 Ab4-B

| | | |
|---|---|---|
| 73 | Heavy Chain | EVQLVESGGGLVKPGGSLRLSCAASGFTFSRTGMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSSASTKGP |
| | | SVFPLAPCSRSTSESTAALGCLVKDYFPEPVTVSWNSGALTSGV |
| | | HTFPAVLQSSGLYSLSSVVTVPSSSLGTKTYTCNVDHKPSNTKV |
| | | DKRVESKYGPPCPSCPAPEFLGGPSVFLFPPKPKDTLMISRTPE |
| | | VTCVVVDVSQEDPEVQFNWYVDGVEVHNAKTKPREEQFNSTYRV |
| | | VSVLTVLHQDWLNGKEYKCKVSNKGLPSSIEKTISKAKGQPREP |
| | | QVYTLPPSQEEMTKNQVSLTCLVKGFYPSDIAVEWESNGQPENN |
| | | YKTTPPVLDSDGSFFLYSRLTVDKSRWQEGNVFSCSVMHEALHN |
| | | HYTQKSLSLSLG |
| 74 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCA |
| | | GTAGGACTGGGATGAACTGGGTCCGCCAGGCTCCAGGGAAGGGG |
| | | CTGGAATGGGTCTCATCCATTAGTAGTAGTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCTTCCACCAAGGGCCCC |
| | | TCCGTGTTCCCTCTGGCCCCTTGCTCCCGGTCCACCTCCGAGTC |
| | | TACCGCCGCTCTGGGCTGCCTCGTGAAGGACTACTTCCCCGAGC |
| | | CCGTGACCGTGTCCTGGAACTCTGGCGCCCTGACCTCCGGCGTG |
| | | CACACCTTCCCTGCCGTGCTGCAGTCCTCCGGCCTGTACTCCCT |
| | | GTCCAGCGTCGTGACCGTGCCCTCCTCCAGCCTGGGCACCAAGA |
| | | CCTACACCTGTAACGTGGACCACAAGCCCTCCAACACCAAAGTG |
| | | GACAAGCGGGTGGAATCTAAGTACGGCCCTCCCTGCCCTTCCTG |
| | | CCCTGCCCCTGAGTTCCTGGGCGGACCTTCCGTGTTCCTGTTCC |
| | | CTCCAAAGCCCAAGGACACCCTGATGATCTCCCGGACCCCTGAA |
| | | GTGACCTGCGTGGTGGTGGACGTGTCCCAGGAAGATCCCGAAGT |
| | | CCAGTTCAATTGGTACGTGGACGGCGTGGAAGTGCACAACGCCA |
| | | AGACCAAGCCCAGAGAGGAACAGTTCAACTCCACCTACCGGGTG |
| | | GTGTCCGTGCTGACCGTGCTGCACCAGGACTGGCTGAACGGCAA |
| | | AGAGTACAAGTGCAAAGTGTCCAACAAGGGCCTGCCCTCCAGCA |
| | | TCGAAAAGACCATCTCCAAGGCCAAGGGCCAGCCCCGCGAGCCC |
| | | CAAGTGTACACCCTGCCTCCCAGCCAGGAAGAGATGACCAAGAA |
| | | TCAAGTGTCCCTGACTTGTCTGGTCAAGGGCTTCTACCCCTCCG |
| | | ATATCGCCGTGGAGTGGGAGTCCAACGGCCAGCCCGAGAACAAC |
| | | TACAAGACCACCCCTCCCGTGCTGGACTCCGACGGCTCCTTCTT |
| | | CCTGTACTCTCGGCTGACCGTGGACAAGTCCCGGTGGCAGGAAG |
| | | GCAACGTCTTCTCCTGCTCCGTGATGCACGAGGCCCTGCACAAC |
| | | CACTACACCCAGAAGTCCCTGTCCCTGTCTCTGGGC |

anti-IL-27 Ab5-A

| | | |
|---|---|---|
| 75 | HCDR1 | GFTFSRYG |
| | (IMGT) | |
| 76 | HCDR2 | ISSSSAYI |
| | (IMGT) | |
| 77 | HCDR3 | ARDGGRTSYTATAHNWFDP |
| | (IMGT) | |
| 78 | HCDR1 (NT) | FTFSRYGMN |
| 79 | HCDR2 (NT) | SISSSSAYILYADSVKG |
| 80 | HCDR3 (NT) | ARDGGRTSYTATAHNWFDP |
| 81 | VH | EVQLVESGGGLVKPGGSLRLSCAASGFTFSRYGMNWVRQAPGKG |
| | | LEWVSSISSSSAYILYADSVKGRFTISRDNAKNSLYLQMNSLRA |
| | | EDTAVYYCARDGGRTSYTATAHNWFDPWGQGTLVTVSS |
| 82 | DNA VH | GAGGTGCAGCTGGTGGAGTCTGGGGGAGGCCTGGTCAAGCCTGG |
| | | GGGGTCCCTGAGACTCTCCTGTGCAGCCTCTGGATTCACCTTCA |
| | | GTAGGTATGGGATGAACTGGGTCCGCCAGGCTCCAGGGAAGGGG |
| | | CTGGAGTGGGTCTCATCCATTAGTAGTAGTAGTGCTTACATACT |
| | | GTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCA |
| 83 | LCDR1 | QSVLFSSNNKNY |
| | (IMGT) | |
| 84 | LCDR2 | WAS |
| | (IMGT) | |
| 85 | LCDR3 | QQHASAPPT |
| | (IMGT) | |
| 86 | LCDR1 (NT) | KSSQSVLFSSNNKNYLA |
| 87 | LCDR2 (NT) | WASTRES |
| 88 | LCDR3 (NT) | QQHASAPPT |
| 89 | VL | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIK |
| 90 | DNA VL | GACATCGTGATGACCCAGTCTCCAGACTCCCTGGCTGTGTCTCT |
| | | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAA |
| 91 | Heavy Chain | EVQLVESGGGLVKPGGSLRLSCAASGFTFSRYGMNWVRQAPGKG |
| | | LEWVSSISSSSAYILYADSVKGRFTISRDNAKNSLYLQMNSLRA |
| | | EDTAVYYCARDGGRTSYTATAHNWFDPWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISR |
| | | TPEVTCVVVDVSHEDPEVKFNWYVDGVEVHNAKTKPREEQYNST |
| | | YRVVSVLTVLHQDWLNGKEYKCKVSNKALPAPIEKTISKAKGQP |
| | | REPQVYTLPPSRDELTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 92 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCA |
| | | GTAGGTATGGGATGAACTGGGTCCGCCAGGCTCCAGGGAAGGGG |
| | | CTGGAGTGGGTCTCATCCATTAGTAGTAGTAGTGCTTACATACT |
| | | GTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCGAGCACCAAAGGCCCG |
| | | AGCGTGTTTCCGCTGGCGCCGAGCAGCAAAAGCACCAGCGGCGG |
| | | CACCGCGGCGCTGGGCTGCCTGGTGAAAGATTATTTTCCGGAAC |
| | | CGGTGACCGTGAGCTGGAACAGCGGCGCGCTGACCAGCGGCGTG |
| | | CATACCTTTCCGGCGGTGCTGCAGAGCAGCGGCCTGTATAGCCT |
| | | GAGCAGCGTGGTGACCGTGCCGAGCAGCAGCCTGGGCACCCAGA |
| | | CCTATATTTGCAACGTGAACCATAAACCGAGCAACACCAAAGTG |
| | | GATAAAAAAGTGGAACCGAAAAGCTGCGATAAAACCCATACCTG |
| | | CCCGCCGTGCCCGGCGCCGGAACTGCTGGGCGGCCCGAGCGTGT |
| | | TTCTGTTTCCGCCGAAACCGAAAGATACCCTGATGATTAGCCGC |
| | | ACCCCGGAAGTGACCTGCGTGGTGGTGGATGTGAGCCATGAAGA |
| | | TCCGGAAGTGAAATTTAACTGGTATGTGGATGGCGTGGAAGTGC |
| | | ATAACGCGAAAACCAAACCGCGCGAAGAACAGTATAACAGCACC |
| | | TATCGCGTGGTGAGCGTGCTGACCGTGCTGCATCAGGATTGGCT |
| | | GAACGGCAAAGAATATAAATGCAAAGTGAGCAACAAAGCGCTGC |
| | | CGGCGCCGATTGAAAAAACCATTAGCAAAGCGAAAGGCCAGCCG |
| | | CGCGAACCGCAGGTGTATACCCTGCCGCCGAGCCGCGATGAACT |
| | | GACCAAAAACCAGGTGAGCCTGACCTGCCTGGTGAAAGGCTTTT |
| | | ATCCGAGCGATATTGCGGTGGAATGGGAAAGCAACGGCCAGCCG |
| | | GAAAACAACTATAAAACCACCCCGCCGGTGCTGGATAGCGATGG |
| | | CAGCTTTTTTCTGTATAGCAAACTGACCGTGGATAAAAGCCGCT |
| | | GGCAGCAGGGCAACGTGTTTAGCTGCAGCGTGATGCATGAAGCG |
| | | CTGCATAACCATTATACCCAGAAAAGCCTGAGCCTGAGCCCGGG |
| | | CAAA |
| 93 | Light Chain | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIKRTVAAPSVFIFPPSDEQLK |
| | | SGTASVVCLLNNFYPREAKVQWKVDNALQSGNSQESVTEQDSKD |
| | | STYSLSSTLTLSKADYEKHKVYACEVTHQGLSSPVTKSFNRGEC |
| 94 | DNA Light | GACATCGTGATGACCCAGTCTCCAGACTCCCTGGCTGTGTCTCT |
| | Chain | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAACGTACGGTGGCCG |
| | | CTCCCTCCGTGTTCATCTTCCCACCCTCCGACGAGCAGCTGAAG |
| | | TCCGGCACCGCCTCCGTCGTGTGCCTGCTGAACAACTTCTACCC |
| | | TCGCGAGGCCAAAGTGCAGTGGAAAGTGGACAACGCCCTGCAGT |
| | | CCGGCAACTCCCAGGAATCCGTCACCGAGCAGGACTCCAAGGAC |
| | | AGCACCTACTCCCTGTCCTCCACCCTGACCCTGTCCAAGGCCGA |
| | | CTACGAGAAGCACAAAGTGTACGCCTGCGAAGTGACCCACCAGG |
| | | GCCTGTCCAGCCCCGTGACCAAGTCCTTCAACCGGGGCGAGTGC |

anti-IL-27 Ab5-B

| | | |
|---|---|---|
| 95 | Heavy Chain | EVQLVESGGGLVKPGGSLRLSCAASGFTFSRYGMNWVRQAPGKG |
| | | LEWVSSISSSSAYILYADSVKGRFTISRDNAKNSLYLQMNSLRA |
| | | EDTAVYYCARDGGRTSYTATAHNWFDPWGQGTLVTVSSASTKGP |
| | | SVFPLAPCSRSTSESTAALGCLVKDYFPEPVTVSWNSGALTSGV |
| | | HTFPAVLQSSGLYSLSSVVTVPSSSLGTKTYTCNVDHKPSNTKV |
| | | DKRVESKYGPPCPSCPAPEFLGGPSVFLFPPKPKDTLMISRTPE |
| | | VTCVVVDVSQEDPEVQFNWYVDGVEVHNAKTKPREEQFNSTYRV |
| | | VSVLTVLHQDWLNGKEYKCKVSNKGLPSSIEKTISKAKGQPREP |
| | | QVYTLPPSQEEMTKNQVSLTCLVKGFYPSDIAVEWESNGQPENN |
| | | YKTTPPVLDSDGSFFLYSRLTVDKSRWQEGNVFSCSVMHEALHN |
| | | HYTQKSLSLSLG |
| 96 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCA |
| | | GTAGGTATGGGATGAACTGGGTCCGCCAGGCTCCAGGGAAGGGG |
| | | CTGGAGTGGGTCTCATCCATTAGTAGTAGTAGTGCTTACATACT |
| | | GTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCTTCCACCAAGGGCCCC |
| | | TCCGTGTTCCCTCTGGCCCCTTGCTCCCGGTCCACCTCCGAGTC |
| | | TACCGCCGCTCTGGGCTGCCTCGTGAAGGACTACTTCCCCGAGC |
| | | CCGTGACCGTGTCCTGGAACTCTGGCGCCCTGACCTCCGGCGTG |
| | | CACACCTTCCCTGCCGTGCTGCAGTCCTCCGGCCTGTACTCCCT |
| | | GTCCAGCGTCGTGACCGTGCCCTCCTCCAGCCTGGGCACCAAGA |
| | | CCTACACCTGTAACGTGGACCACAAGCCCTCCAACACCAAAGTG |
| | | GACAAGCGGGTGGAATCTAAGTACGGCCCTCCCTGCCCTTCCTG |
| | | CCCTGCCCCTGAGTTCCTGGGCGGACCTTCCGTGTTCCTGTTCC |
| | | CTCCAAAGCCCAAGGACACCCTGATGATCTCCCGGACCCCTGAA |
| | | GTGACCTGCGTGGTGGTGGACGTGTCCCAGGAAGATCCCGAAGT |
| | | CCAGTTCAATTGGTACGTGGACGGCGTGGAAGTGCACAACGCCA |
| | | AGACCAAGCCCAGAGAGGAACAGTTCAACTCCACCTACCGGGTG |
| | | GTGTCCGTGCTGACCGTGCTGCACCAGGACTGGCTGAACGGCAA |
| | | AGAGTACAAGTGCAAAGTGTCCAACAAGGGCCTGCCCTCCAGCA |
| | | TCGAAAAGACCATCTCCAAGGCCAAGGGCCAGCCCCGCGAGCCC |
| | | CAAGTGTACACCCTGCCTCCCAGCCAGGAAGAGATGACCAAGAA |
| | | TCAAGTGTCCCTGACTTGTCTGGTCAAGGGCTTCTACCCCTCCG |
| | | ATATCGCCGTGGAGTGGGAGTCCAACGGCCAGCCCGAGAACAAC |
| | | TACAAGACCACCCCTCCCGTGCTGGACTCCGACGGCTCCTTCTT |
| | | CCTGTACTCTCGGCTGACCGTGGACAAGTCCCGGTGGCAGGAAG |
| | | GCAACGTCTTCTCCTGCTCCGTGATGCACGAGGCCCTGCACAAC |
| | | CACTACACCCAGAAGTCCCTGTCCCTGTCTCTGGGC |

anti-IL-27 Ab6-A

| | | |
|---|---|---|
| 97 | HCDR1 | GFTFASYG |
| | (IMGT) | |
| 98 | HCDR2 | ISSSSSYI |
| | (IMGT) | |
| 99 | HCDR3 | ARDGGRTSYTATAHNWFDP |
| | (IMGT) | |
| 100 | HCDR1 (NT) | FTFASYGMN |
| 101 | HCDR2 (NT) | SISSSSSYIYYADSVKG |
| 102 | HCDR3 (NT) | ARDGGRTSYTATAHNWFDP |
| 103 | VH | EVQLVESGGGLVKPGGSLRLSCAASGFTFASYGMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSS |
| 104 | DNA VH | GAGGTGCAGCTGGTGGAGTCTGGGGGAGGCCTGGTCAAGCCTGG |
| | | GGGGTCCCTGAGACTCTCCTGTGCAGCCTCTGGATTCACCTTCG |
| | | CTAGCTATGGGATGAACTGGGTCCGCCAGGCTCCAGGGAAGGGG |
| | | CTGGAGTGGGTCTCATCCATTAGTAGTTCTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCA |
| 105 | LCDR1 | QSVLFSSNNKNY |
| | (IMGT) | |
| 106 | LCDR2 | WAS |
| | (IMGT) | |
| 107 | LCDR3 | QQHASAPPT |
| | (IMGT) | |
| 108 | LCDR1 (NT) | KSSQSVLFSSNNKNYLA |
| 109 | LCDR2 (NT) | WASTRES |
| 110 | LCDR3 (NT) | QQHASAPPT |
| 111 | VL | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIK |
| 112 | DNA VL | GACATCGTGATGACCCAGTCTCCAGACTCCCTGGCTGTGTCTCT |
| | | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAA |
| 113 | Heavy Chain | EVQLVESGGGLVKPGGSLRLSCAASGFTFASYGMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISR |
| | | TPEVTCVVVDVSHEDPEVKFNWYVDGVEVHNAKTKPREEQYNST |
| | | YRVVSVLTVLHQDWLNGKEYKCKVSNKALPAPIEKTISKAKGQP |
| | | REPQVYTLPPSRDELTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 114 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCG |
| | | CTAGCTATGGGATGAACTGGGTCCGCCAGGCTCCAGGGAAGGGG |
| | | CTGGAGTGGGTCTCATCCATTAGTAGTTCTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCGAGCACCAAAGGCCCG |
| | | AGCGTGTTTCCGCTGGCGCCGAGCAGCAAAAGCACCAGCGGCGG |
| | | CACCGCGGCGCTGGGCTGCCTGGTGAAAGATTATTTTCCGGAAC |
| | | CGGTGACCGTGAGCTGGAACAGCGGCGCGCTGACCAGCGGCGTG |
| | | CATACCTTTCCGGCGGTGCTGCAGAGCAGCGGCCTGTATAGCCT |
| | | GAGCAGCGTGGTGACCGTGCCGAGCAGCAGCCTGGGCACCCAGA |
| | | CCTATATTTGCAACGTGAACCATAAACCGAGCAACACCAAAGTG |
| | | GATAAAAAAGTGGAACCGAAAAGCTGCGATAAAACCCATACCTG |
| | | CCCGCCGTGCCCGGCGCCGGAACTGCTGGGCGGCCCGAGCGTGT |
| | | TTCTGTTTCCGCCGAAACCGAAAGATACCCTGATGATTAGCCGC |
| | | ACCCCGGAAGTGACCTGCGTGGTGGTGGATGTGAGCCATGAAGA |
| | | TCCGGAAGTGAAATTTAACTGGTATGTGGATGGCGTGGAAGTGC |
| | | ATAACGCGAAAACCAAACCGCGCGAAGAACAGTATAACAGCACC |
| | | TATCGCGTGGTGAGCGTGCTGACCGTGCTGCATCAGGATTGGCT |
| | | GAACGGCAAAGAATATAAATGCAAAGTGAGCAACAAAGCGCTGC |
| | | CGGCGCCGATTGAAAAAACCATTAGCAAAGCGAAAGGCCAGCCG |
| | | CGCGAACCGCAGGTGTATACCCTGCCGCCGAGCCGCGATGAACT |
| | | GACCAAAAACCAGGTGAGCCTGACCTGCCTGGTGAAAGGCTTTT |
| | | ATCCGAGCGATATTGCGGTGGAATGGGAAAGCAACGGCCAGCCG |
| | | GAAAACAACTATAAAACCACCCCGCCGGTGCTGGATAGCGATGG |
| | | CAGCTTTTTTCTGTATAGCAAACTGACCGTGGATAAAAGCCGCT |
| | | GGCAGCAGGGCAACGTGTTTAGCTGCAGCGTGATGCATGAAGCG |
| | | CTGCATAACCATTATACCCAGAAAAGCCTGAGCCTGAGCCCGGG |
| | | CAAA |
| 115 | Light Chain | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIKRTVAAPSVFIFPPSDEQLK |
| | | SGTASVVCLLNNFYPREAKVQWKVDNALQSGNSQESVTEQDSKD |
| | | STYSLSSTLTLSKADYEKHKVYACEVTHQGLSSPVTKSFNRGEC |
| 116 | DNA Light | GACATCGTGATGACCCAGTCTCCAGACTCCCTGGCTGTGTCTCT |
| | Chain | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAACGTACGGTGGCCG |
| | | CTCCCTCCGTGTTCATCTTCCCACCCTCCGACGAGCAGCTGAAG |
| | | TCCGGCACCGCCTCCGTCGTGTGCCTGCTGAACAACTTCTACCC |
| | | TCGCGAGGCCAAAGTGCAGTGGAAAGTGGACAACGCCCTGCAGT |
| | | CCGGCAACTCCCAGGAATCCGTCACCGAGCAGGACTCCAAGGAC |
| | | AGCACCTACTCCCTGTCCTCCACCCTGACCCTGTCCAAGGCCGA |
| | | CTACGAGAAGCACAAAGTGTACGCCTGCGAAGTGACCCACCAGG |
| | | GCCTGTCCAGCCCCGTGACCAAGTCCTTCAACCGGGGCGAGTGC |

anti-IL-27 Ab6-B

| | | |
|---|---|---|
| 117 | Heavy Chain | EVQLVESGGGLVKPGGSLRLSCAASGFTFASYGMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSSASTKGP |
| | | SVFPLAPCSRSTSESTAALGCLVKDYFPEPVTVSWNSGALTSGV |
| | | HTFPAVLQSSGLYSLSSVVTVPSSSLGTKTYTCNVDHKPSNTKV |
| | | DKRVESKYGPPCPSCPAPEFLGGPSVFLFPPKPKDTLMISRTPE |
| | | VTCVVVDVSQEDPEVQFNWYVDGVEVHNAKTKPREEQFNSTYRV |
| | | VSVLTVLHQDWLNGKEYKCKVSNKGLPSSIEKTISKAKGQPREP |
| | | QVYTLPPSQEEMTKNQVSLTCLVKGFYPSDIAVEWESNGQPENN |
| | | YKTTPPVLDSDGSFFLYSRLTVDKSRWQEGNVFSCSVMHEALHN |
| | | HYTQKSLSLSLG |
| 118 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCG |
| | | CTAGCTATGGGATGAACTGGGTCCGCCAGGCTCCAGGGAAGGGG |
| | | CTGGAGTGGGTCTCATCCATTAGTAGTTCTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCTTCCACCAAGGGCCCC |
| | | TCCGTGTTCCCTCTGGCCCCTTGCTCCCGGTCCACCTCCGAGTC |
| | | TACCGCCGCTCTGGGCTGCCTCGTGAAGGACTACTTCCCCGAGC |
| | | CCGTGACCGTGTCCTGGAACTCTGGCGCCCTGACCTCCGGCGTG |
| | | CACACCTTCCCTGCCGTGCTGCAGTCCTCCGGCCTGTACTCCCT |
| | | GTCCAGCGTCGTGACCGTGCCCTCCTCCAGCCTGGGCACCAAGA |
| | | CCTACACCTGTAACGTGGACCACAAGCCCTCCAACACCAAAGTG |
| | | GACAAGCGGGTGGAATCTAAGTACGGCCCTCCCTGCCCTTCCTG |
| | | CCCTGCCCCTGAGTTCCTGGGCGGACCTTCCGTGTTCCTGTTCC |
| | | CTCCAAAGCCCAAGGACACCCTGATGATCTCCCGGACCCCTGAA |
| | | GTGACCTGCGTGGTGGTGGACGTGTCCCAGGAAGATCCCGAAGT |
| | | CCAGTTCAATTGGTACGTGGACGGCGTGGAAGTGCACAACGCCA |
| | | AGACCAAGCCCAGAGAGGAACAGTTCAACTCCACCTACCGGGTG |
| | | GTGTCCGTGCTGACCGTGCTGCACCAGGACTGGCTGAACGGCAA |
| | | AGAGTACAAGTGCAAAGTGTCCAACAAGGGCCTGCCCTCCAGCA |
| | | TCGAAAAGACCATCTCCAAGGCCAAGGGCCAGCCCCGCGAGCCC |
| | | CAAGTGTACACCCTGCCTCCCAGCCAGGAAGAGATGACCAAGAA |
| | | TCAAGTGTCCCTGACTTGTCTGGTCAAGGGCTTCTACCCCTCCG |
| | | ATATCGCCGTGGAGTGGGAGTCCAACGGCCAGCCCGAGAACAAC |
| | | TACAAGACCACCCCTCCCGTGCTGGACTCCGACGGCTCCTTCTT |
| | | CCTGTACTCTCGGCTGACCGTGGACAAGTCCCGGTGGCAGGAAG |
| | | GCAACGTCTTCTCCTGCTCCGTGATGCACGAGGCCCTGCACAAC |
| | | CACTACACCCAGAAGTCCCTGTCCCTGTCTCTGGGC |

anti-IL-27 Ab1-A

| | | |
|---|---|---|
| 119 | HCDR1 | GFTFRSYG |
| | (IMGT) | |
| 120 | HCDR2 | ISSSGSYI |
| | (IMGT) | |
| 121 | HCDR3 | ARDGGRTSYTATAHNWFDP |
| | (IMGT) | |
| 122 | HCDR1 (NT) | FTFRSYGMN |
| 123 | HCDR2 (NT) | GISSSGSYIYYADSVKG |
| 124 | HCDR3 (NT) | ARDGGRTSYTATAHNWFDP |
| 125 | VH | EVQLVESGGGLVKPGGSLRLSCAASGFTFRSYGMNWVRQAPGKG |
| | | LEWVSGISSSGSYIYYADSVKGRFTISRDNAKNSLYLQMNSLRA |
| | | EDTAVYYCARDGGRTSYTATAHNWFDPWGQGTLVTVSS |
| 126 | DNA VH | GAGGTGCAGCTGGTGGAGTCTGGGGGAGGCCTGGTCAAGCCTGG |
| | | GGGGTCCCTGAGACTCTCCTGTGCAGCCTCTGGATTCACCTTCC |
| | | GTAGCTATGGGATGAACTGGGTCCGCCAGGCTCCAGGGAAGGGG |
| | | CTGGAGTGGGTCTCAGGTATTAGTAGTAGTGGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCA |
| 127 | LCDR1 | QSVLFSSNNKNY |
| | (IMGT) | |
| 128 | LCDR2 | WAS |
| | (IMGT) | |
| 129 | LCDR3 | QQHASAPPT |
| | (IMGT) | |
| 130 | LCDR1 (NT) | KSSQSVLFSSNNKNYLA |
| 131 | LCDR2 (NT) | WASTRES |
| 132 | LCDR3 (NT) | QQHASAPPT |
| 133 | VL | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIK |
| 134 | DNA VL | GACATCGTGATGACCCAGTCTCCAGACTCCCTGGCTGTGTCTCT |
| | | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAA |
| 135 | Heavy Chain | EVQLVESGGGLVKPGGSLRLSCAASGFTFRSYGMNWVRQAPGKG |
| | | LEWVSGISSSGSYIYYADSVKGRFTISRDNAKNSLYLQMNSLRA |
| | | EDTAVYYCARDGGRTSYTATAHNWFDPWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISR |
| | | TPEVTCVVVDVSHEDPEVKFNWYVDGVEVHNAKTKPREEQYNST |
| | | YRVVSVLTVLHQDWLNGKEYKCKVSNKALPAPIEKTISKAKGQP |
| | | REPQVYTLPPSRDELTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 136 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCC |
| | | GTAGCTATGGGATGAACTGGGTCCGCCAGGCTCCAGGGAAGGGG |
| | | CTGGAGTGGGTCTCAGGTATTAGTAGTAGTGGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCGAGCACCAAAGGCCCG |
| | | AGCGTGTTTCCGCTGGCGCCGAGCAGCAAAAGCACCAGCGGCGG |
| | | CACCGCGGCGCTGGGCTGCCTGGTGAAAGATTATTTTCCGGAAC |
| | | CGGTGACCGTGAGCTGGAACAGCGGCGCGCTGACCAGCGGCGTG |
| | | CATACCTTTCCGGCGGTGCTGCAGAGCAGCGGCCTGTATAGCCT |
| | | GAGCAGCGTGGTGACCGTGCCGAGCAGCAGCCTGGGCACCCAGA |
| | | CCTATATTTGCAACGTGAACCATAAACCGAGCAACACCAAAGTG |
| | | GATAAAAAAGTGGAACCGAAAAGCTGCGATAAAACCCATACCTG |
| | | CCCGCCGTGCCCGGCGCCGGAACTGCTGGGCGGCCCGAGCGTGT |
| | | TTCTGTTTCCGCCGAAACCGAAAGATACCCTGATGATTAGCCGC |
| | | ACCCCGGAAGTGACCTGCGTGGTGGTGGATGTGAGCCATGAAGA |
| | | TCCGGAAGTGAAATTTAACTGGTATGTGGATGGCGTGGAAGTGC |
| | | ATAACGCGAAAACCAAACCGCGCGAAGAACAGTATAACAGCACC |
| | | TATCGCGTGGTGAGCGTGCTGACCGTGCTGCATCAGGATTGGCT |
| | | GAACGGCAAAGAATATAAATGCAAAGTGAGCAACAAAGCGCTGC |
| | | CGGCGCCGATTGAAAAAACCATTAGCAAAGCGAAAGGCCAGCCG |
| | | CGCGAACCGCAGGTGTATACCCTGCCGCCGAGCCGCGATGAACT |
| | | GACCAAAAACCAGGTGAGCCTGACCTGCCTGGTGAAAGGCTTTT |
| | | ATCCGAGCGATATTGCGGTGGAATGGGAAAGCAACGGCCAGCCG |
| | | GAAAACAACTATAAAACCACCCCGCCGGTGCTGGATAGCGATGG |
| | | CAGCTTTTTTCTGTATAGCAAACTGACCGTGGATAAAAGCCGCT |
| | | GGCAGCAGGGCAACGTGTTTAGCTGCAGCGTGATGCATGAAGCG |
| | | CTGCATAACCATTATACCCAGAAAAGCCTGAGCCTGAGCCCGGG |
| | | CAAA |
| 137 | Light Chain | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIKRTVAAPSVFIFPPSDEQLK |
| | | SGTASVVCLLNNFYPREAKVQWKVDNALQSGNSQESVTEQDSKD |
| | | STYSLSSTLTLSKADYEKHKVYACEVTHQGLSSPVTKSFNRGEC |
| 138 | DNA Light | GACATCGTGATGACCCAGTCTCCAGACTCCCTGGCTGTGTCTCT |
| | Chain | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAACGTACGGTGGCCG |
| | | CTCCCTCCGTGTTCATCTTCCCACCCTCCGACGAGCAGCTGAAG |
| | | TCCGGCACCGCCTCCGTCGTGTGCCTGCTGAACAACTTCTACCC |
| | | TCGCGAGGCCAAAGTGCAGTGGAAAGTGGACAACGCCCTGCAGT |
| | | CCGGCAACTCCCAGGAATCCGTCACCGAGCAGGACTCCAAGGAC |
| | | AGCACCTACTCCCTGTCCTCCACCCTGACCCTGTCCAAGGCCGA |
| | | CTACGAGAAGCACAAAGTGTACGCCTGCGAAGTGACCCACCAGG |
| | | GCCTGTCCAGCCCCGTGACCAAGTCCTTCAACCGGGGCGAGTGC |

anti-IL-27 Ab1-B

| | | |
|---|---|---|
| 139 | Heavy Chain | EVQLVESGGGLVKPGGSLRLSCAASGFTFRSYGMNWVRQAPGKG |
| 140 | DNA Heavy | LEWVSGISSSGSYIYYADSVKGRFTISRDNAKNSLYLQMNSLRA |
| | Chain | EDTAVYYCARDGGRTSYTATAHNWFDPWGQGTLVTVSSASTKGP |
| | | SVFPLAPCSRSTSESTAALGCLVKDYFPEPVTVSWNSGALTSGV |
| | | HTFPAVLQSSGLYSLSSVVTVPSSSLGTKTYTCNVDHKPSNTKV |
| | | DKRVESKYGPPCPSCPAPEFLGGPSVFLFPPKPKDTLMISRTPE |
| | | VTCVVVDVSQEDPEVQFNWYVDGVEVHNAKTKPREEQFNSTYRV |
| | | VSVLTVLHQDWLNGKEYKCKVSNKGLPSSIEKTISKAKGQPREP |
| | | QVYTLPPSQEEMTKNQVSLTCLVKGFYPSDIAVEWESNGQPENN |
| | | YKTTPPVLDSDGSFFLYSRLTVDKSRWQEGNVFSCSVMHEALHN |
| | | HYTQKSLSLSLG |
| | | GAGGTGCAGCTGGTGGAGTCTGGGGGAGGCCTGGTCAAGCCTGG |
| | | GGGGTCCCTGAGACTCTCCTGTGCAGCCTCTGGATTCACCTTCC |
| | | GTAGCTATGGGATGAACTGGGTCCGCCAGGCTCCAGGGAAGGGG |
| | | CTGGAGTGGGTCTCAGGTATTAGTAGTAGTGGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCTTCCACCAAGGGCCCC |
| | | TCCGTGTTCCCTCTGGCCCCTTGCTCCCGGTCCACCTCCGAGTC |
| | | TACCGCCGCTCTGGGCTGCCTCGTGAAGGACTACTTCCCCGAGC |
| | | CCGTGACCGTGTCCTGGAACTCTGGCGCCCTGACCTCCGGCGTG |
| | | CACACCTTCCCTGCCGTGCTGCAGTCCTCCGGCCTGTACTCCCT |
| | | GTCCAGCGTCGTGACCGTGCCCTCCTCCAGCCTGGGCACCAAGA |
| | | CCTACACCTGTAACGTGGACCACAAGCCCTCCAACACCAAAGTG |
| | | GACAAGCGGGTGGAATCTAAGTACGGCCCTCCCTGCCCTTCCTG |
| | | CCCTGCCCCTGAGTTCCTGGGCGGACCTTCCGTGTTCCTGTTCC |
| | | CTCCAAAGCCCAAGGACACCCTGATGATCTCCCGGACCCCTGAA |
| | | GTGACCTGCGTGGTGGTGGACGTGTCCCAGGAAGATCCCGAAGT |
| | | CCAGTTCAATTGGTACGTGGACGGCGTGGAAGTGCACAACGCCA |
| | | AGACCAAGCCCAGAGAGGAACAGTTCAACTCCACCTACCGGGTG |
| | | GTGTCCGTGCTGACCGTGCTGCACCAGGACTGGCTGAACGGCAA |
| | | AGAGTACAAGTGCAAAGTGTCCAACAAGGGCCTGCCCTCCAGCA |
| | | TCGAAAAGACCATCTCCAAGGCCAAGGGCCAGCCCCGCGAGCCC |
| | | CAAGTGTACACCCTGCCTCCCAGCCAGGAAGAGATGACCAAGAA |
| | | TCAAGTGTCCCTGACTTGTCTGGTCAAGGGCTTCTACCCCTCCG |
| | | ATATCGCCGTGGAGTGGGAGTCCAACGGCCAGCCCGAGAACAAC |
| | | TACAAGACCACCCCTCCCGTGCTGGACTCCGACGGCTCCTTCTT |
| | | CCTGTACTCTCGGCTGACCGTGGACAAGTCCCGGTGGCAGGAAG |
| | | GCAACGTCTTCTCCTGCTCCGTGATGCACGAGGCCCTGCACAAC |
| | | CACTACACCCAGAAGTCCCTGTCCCTGTCTCTGGGC |
| 141 | FLAG | DYKDDDDK |
| 142 | 6-HIS | HHHHHH |
| 143 | HA | YPYDVPDYA |

In some aspects, the antibody, or antigen binding portion thereof, comprises an Fc sequence set forth in Table 1B. In some aspects, the antibody, or antigen binding portion thereof, comprises a heavy chain, wherein the heavy chain comprises an Fc region having an amino acid at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 5, 6, 7, or 8. In some aspects, the antibody, or antigen binding portion thereof, comprises a heavy chain, wherein the heavy chain comprises an Fc region comprising the amino acid sequence set forth in SEQ ID NO: 5. In some aspects, the antibody, or antigen binding portion thereof, comprises a heavy chain, wherein the heavy chain comprises an Fc region comprising the amino acid sequence set forth in SEQ ID NO: 6. In some aspects, the antibody, or antigen binding portion thereof, comprises a heavy chain, wherein the heavy chain comprises an Fc region comprising the amino acid sequence set forth in SEQ ID NO: 7. In some aspects, the antibody, or antigen binding portion thereof, comprises a heavy chain, wherein the heavy chain comprises an Fc region comprising the amino acid sequence set forth in SEQ ID NO: 8.

TABLE 1B
Fc Sequences (═CH2+CH3)

| Name | Alias | Amino Acid Sequence |
|---|---|---|
| Human | 1.0 | EPKSCDKTHTCPPCPAPELLGGPSVFLFPPKPKDTLMISRTPEVTC |
| IgG1 | | VVVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPP |
| | | SRDELTKNQVSLTCLVKGFYPSDIAVEWESNGQPENNYKTTPPVLD |
| | | SDGSFFLYSKLTVDKSRWQQGNVFSCSVMHEALHNHYTQKSLSLSP |
| | | GK (SEQ ID NO: 5) |
| Human | 4.0 | ESKYGPPCPSCPAPEFLGGPSVFLFPPKPKDTLMISRTPEVTCVVV |
| IgG4 | | DVSQEDPEVQFNWYVDGVEVHNAKTKPREEQFNSTYRVVSVLTVLH |
| | | QDWLNGKEYKCKVSNKGLPSSIEKTISKAKGQPREPQVYTLPPSQE |
| | | EMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK |
| | | (SEQ ID NO: 6) |
| Human | 4.1 | ESKYGPPCPPCPAPEFLGGPSVFLFPPKPKDTLMISRTPEVTCVVV |
| IgG4 | | DVSQEDPEVQFNWYVDGVEVHNAKTKPREEQFNSTYRVVSVLTVLH |
| (S228P) | | QDWLNGKEYKCKVSNKGLPSSIEKTISKAKGQPREPQVYTLPPSQE |
| | | EMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK |
| | | (SEQ ID NO: 7) |
| Human | 4.2 | ESKYGPPCPPCPAPEFEGGPSVFLFPPKPKDTLMISRTPEVTCVVV |
| IgG4 | | DVSQEDPEVQFNWYVDGVEVHNAKTKPREEQFNSTYRVVSVLTVLH |
| (S228P / | | QDWLNGKEYKCKVSNKGLPSSIEKTISKAKGQPREPQVYTLPPSQE |
| L235E) | | EMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK |
| | | (SEQ ID NO: 8) |

In some aspects, an antibody, or antigen binding portion thereof, of the present disclosure specifically binds to an epitope comprising Asp146 and Arg149 of SEQ ID NO: 2 (IL-27p28). In some aspects, an antibody, or antigen binding portion thereof, of the present disclosure specifically binds to an epitope comprising Asp146 and Phe153 of SEQ ID NO: 2 (IL-27p28). In some aspects, an antibody, or antigen binding portion thereof, of the present disclosure specifically binds to an epitope comprising Arg149 and Phe153 of SEQ ID NO: 2 (IL-27p28). In some aspects, an antibody, or antigen binding portion thereof, of the present disclosure specifically binds to an epitope comprising Asp146, Arg149, and/or Phe153 of SEQ ID NO: 2 (IL-27p28). In some aspects, an antibody, or antigen binding portion thereof, of the present disclosure specifically binds to an epitope comprising Asp146, Arg149, and Phe153 of SEQ ID NO: 2 (IL-27p28). In some aspects, the epitope comprises Asp146, Arg149, His150, and Phe153 of SEQ ID NO: 2 (IL-27p28). In some aspects, the epitope comprises Asp146, Arg149, Phe153, and Leu156 of SEQ ID NO: 2 (IL-27p28). In some aspects, the epitope comprises Asp146, Arg149, His150, Phe153, and Leu156 of SEQ ID NO: 2 (IL-27p28).

In some aspects, an antibody, or antigen binding portion thereof, of the present disclosure specifically binds to an epitope comprising at least one, at least two, at least three, at least four, at least five, or at least six amino acids of IL-27p28 selected from Leu142, Asp146, Arg149, His 150, Phe153, Leu156, and Glu164 of SEQ ID NO: 2 (IL-27p28). In some aspects, an antibody, or antigen binding portion thereof, of the present disclosure specifically binds to an epitope comprising Leu142, Asp146, Arg149, His150, Phe153, Leu156, and Glu164 of SEQ ID NO: 2 (IL-27p28). In some aspects, the epitope comprises Gln37, Leu38, Glu42, Asp146, Arg149, His150, Phe153, and Leu156 of SEQ ID NO: 2 (IL-27p28). In some aspects, an antibody, or antigen binding portion thereof, of the present disclosure specifically binds to an epitope comprising Gln37, Leu38, Glu42, Leu142, Asp146, Arg149, His150, Phe153, Leu156, and Glu164 of SEQ ID NO: 2 (IL-27p28).

In some aspects, an antibody, or antigen binding portion thereof, of the present disclosure specifically binds to an epitope comprising Leu142, Asp146, Arg149, His150, Phe153, Leu156, Leu162, and Glu164 of SEQ ID NO: 2 (IL-27p28). In some aspects, an antibody, or antigen binding portion thereof, of the present disclosure specifically binds to an epitope comprising at least one, at least two, at least three, at least four, at least five, or at least six, at least seven, at least eight, or at least nine amino acids of IL-27p28 selected from Glu46, Val49, Ser50, Leu142, Asp146, Arg149, His150, Phe153, Leu156, and Glu164 of SEQ ID NO: 2 (IL-27p28). In some aspects, an antibody, or antigen binding portion thereof, of the present disclosure specifically binds to an epitope comprising Glu46, Val49, Ser50, Leu142, Asp146, Arg149, His150, Phe153, Leu156, and Glu164 of SEQ ID NO: 2 (IL-27p28). In some aspects, an antibody, or antigen binding portion thereof, of the present disclosure specifically binds to an epitope comprising at least one, at least two, at least three, at least four, at least five, or at least six, at least seven, at least eight, or at least nine amino acids of IL-27p28 selected from Gln37, Leu38, Glu42, Glu46, Val49, Ser50, Leu142, Asp146, Arg149, His150, Phe153, Leu156, Leu162, and Glu164 of SEQ ID NO: 2 (IL-27p28). In some aspects, an antibody, or antigen binding portion thereof, of the present disclosure specifically binds to an epitope comprising Gln37, Leu38, Glu42, Glu46, Val49, Ser50, Leu142, Asp146, Arg149, His150, Phe153, Leu156, Leu162, and Glu164 of SEQ ID NO: 2 (IL-27p28).

In some aspects, an antibody, or antigen binding portion thereof, of the present disclosure specifically binds to an epitope consisting of or consisting essentially of Gln37, Leu38, Glu42, Glu46, Val49, Ser50, Leu142, Asp146, Arg149, His150, Phe153, Leu156, Leu162, and Glu164 of SEQ ID NO: 2 (IL-27p28).

In some aspects, an antibody, or antigen binding portion thereof, of the present disclosure specifically binds to an epitope comprising Gln37, Leu38, Glu42, Glu46, Val49, Ser50, Leu142, Asp146, Arg149, His150, Phe153, Leu156, Leu162, and Glu164 of SEQ ID NO: 2 (IL-27p28) and at least one residues selected from the group consisting of: Leu53, Lys56, Asp143, Leu147, Arg152, Ala157, Gly159, Phe160, or Asn161 of SEQ ID NO: 2 (IL-27p28).

In some aspects, an antibody, or antigen binding portion thereof, of the present disclosure specifically binds to an epitope comprising Gln37, Leu38, Glu42, Glu46, Val49, Ser50, Leu142, Asp146, Arg149, His150, Phe153, Leu156, Leu162, and Glu164 of SEQ ID NO: 2 (IL-27p28) and at least one residues selected from the group consisting of: Leu53, Lys56, Asp143, Arg145, Leu147, Arg152, Ala157, Gly159, Phe160, Asn161, or Pro163 of SEQ ID NO: 2 (IL-27p28).

In some aspects, an antibody, or antigen binding portion thereof, of the present disclosure specifically binds to an epitope consisting or consisting essentially of Gln37, Leu38, Glu42, Glu46, Val49, Ser50, Leu53, Lys56, Leu142, Asp143, Asp146, Leu147, Arg149, His150, Arg152, Phe153, Leu156, Ala157, Gly159, Phe160, Asn161, Leu162, and Glu164 of SEQ ID NO: 2 (IL-27p28).

In some aspects, an antibody, or antigen binding portion thereof, of the present disclosure specifically binds to an epitope consisting or consisting essentially of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28).

In some aspects, the disclosure provides an isolated antibody that specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28) and antagonizes human IL-27, or an antigen binding portion thereof, wherein the antibody or antigen binding portion thereof exhibits at least one or more of the following properties: (i) binds to human IL-27 with an equilibrium dissociation constant (K_{D}) of 15 nM or less; (ii) blocks binding of IL-27 to IL-27 receptor; (iii) inhibits or reduces STAT1 and/or STAT3 phosphorylation in a cell; (iv) inhibits or reduces inhibition of CD161 expression in a cell; (v) inhibits or reduces PD-L1 expression in a cell; (vi) induces or enhances PD-1 mediated secretion of one or more cytokines from a cell; (vii) alters TIM-3 expression in a cell; and (viii) a combination of (i)-(vii).

In some aspects, the isolated antibody, or antigen binding portion thereof, binds to an epitope of one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (human IL-27p28) with an equilibrium dissociation constant (K_{D}) of 15 nM or less.

In some aspects, the isolated antibody, or antigen binding portion thereof, binds to recombinant human IL-27p28. In some aspects, the isolated antibody, or antigen binding portion thereof, binds to murine IL-27p28.

In some aspects, the isolated antibody, or antigen binding portion thereof, inhibits or reduces STAT1 phosphorylation in a cell. In some aspects, the isolated antibody, or antigen binding portion thereof, inhibits or reduces STAT3 phosphorylation in a cell. In some aspects, the isolated antibody, or antigen binding portion thereof, inhibits or reduces STAT1 and STAT3 phosphorylation in a cell. In some aspects, the isolated antibody, or antigen binding portion thereof, inhibits or reduces STAT1 phosphorylation in a cell by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%, relative to the STAT1 phosphorylation in the cell prior to contacting the cell with the antibody, or antigen binding portion thereof. In some aspects, the isolated antibody, or antigen binding portion thereof, inhibits or reduces STAT1 phosphorylation in a cell by at least about 50%, relative to the STAT1 phosphorylation in the cell prior to contacting the cell with the antibody, or antigen binding portion thereof. In some aspects, the isolated antibody, or antigen binding portion thereof, inhibits or reduces STAT1 phosphorylation in a cell by at least about 60%, relative to the STAT1 phosphorylation in the cell prior to contacting the cell with the antibody, or antigen binding portion thereof. In some aspects, the isolated antibody, or antigen binding portion thereof, inhibits or reduces STAT1 phosphorylation in a cell by at least about 70%, relative to the STAT1 phosphorylation in the cell prior to contacting the cell with the antibody, or antigen binding portion thereof. In some aspects, the isolated antibody, or antigen binding portion thereof, inhibits or reduces STAT1 phosphorylation in a cell by at least about 75%, relative to the STAT1 phosphorylation in the cell prior to contacting the cell with the antibody, or antigen binding portion thereof. In some aspects, the isolated antibody, or antigen binding portion thereof, inhibits or reduces STAT1 phosphorylation in a cell by at least about 80%, relative to the STAT1 phosphorylation in the cell prior to contacting the cell with the antibody, or antigen binding portion thereof. In some aspects, the isolated antibody, or antigen binding portion thereof, inhibits or reduces STAT1 phosphorylation in a cell by at least about 85%, relative to the STAT1 phosphorylation in the cell prior to contacting the cell with the antibody, or antigen binding portion thereof. In some aspects, the isolated antibody, or antigen binding portion thereof, inhibits or reduces STAT1 phosphorylation in a cell by at least about 90%, relative to the STAT1 phosphorylation in the cell prior to contacting the cell with the antibody, or antigen binding portion thereof. In some aspects, the isolated antibody, or antigen binding portion thereof, inhibits or reduces STAT1 phosphorylation in a cell by at least about 95%, relative to the STAT1 phosphorylation in the cell prior to contacting the cell with the antibody, or antigen binding portion thereof. In some aspects, the isolated antibody, or antigen binding portion thereof, eliminates STAT1 phosphorylation in the cell.

In some aspects, the isolated antibody, or antigen binding portion thereof, inhibits or reduces STAT3 phosphorylation in a cell by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%, relative to the STAT3 phosphorylation in the cell prior to contacting the cell with the antibody, or antigen binding portion thereof. In some aspects, the isolated antibody, or antigen binding portion thereof, inhibits or reduces STAT3 phosphorylation in a cell by at least about 50%, relative to the STAT3 phosphorylation in the cell prior to contacting the cell with the antibody, or antigen binding portion thereof. In some aspects, the isolated antibody, or antigen binding portion thereof, inhibits or reduces STAT3 phosphorylation in a cell by at least about 60%, relative to the STAT3 phosphorylation in the cell prior to contacting the cell with the antibody, or antigen binding portion thereof. In some aspects, the isolated antibody, or antigen binding portion thereof, inhibits or reduces STAT3 phosphorylation in a cell by at least about 70%, relative to the STAT3 phosphorylation in the cell prior to contacting the cell with the antibody, or antigen binding portion thereof. In some aspects, the isolated antibody, or antigen binding portion thereof, inhibits or reduces STAT3 phosphorylation in a cell by at least about 75%, relative to the STAT3 phosphorylation in the cell prior to contacting the cell with the antibody, or antigen binding portion thereof. In some aspects, the isolated antibody, or antigen binding portion thereof, inhibits or reduces STAT3 phosphorylation in a cell by at least about 80%, relative to the STAT3 phosphorylation in the cell prior to contacting the cell with the antibody, or antigen binding portion thereof. In some aspects, the isolated antibody, or antigen binding portion thereof, inhibits or reduces STAT3 phosphorylation in a cell by at least about 85%, relative to the STAT3 phosphorylation in the cell prior to contacting the cell with the antibody, or antigen binding portion thereof. In some aspects, the isolated antibody, or antigen binding portion thereof, inhibits or reduces STAT3 phosphorylation in a cell by at least about 90%, relative to the STAT3 phosphorylation in the cell prior to contacting the cell with the antibody, or antigen binding portion thereof. In some aspects, the isolated antibody, or antigen binding portion thereof, inhibits or reduces STAT3 phosphorylation in a cell by at least about 95%, relative to the STAT3 phosphorylation in the cell prior to contacting the cell with the antibody, or antigen binding portion thereof. In some aspects, the isolated antibody, or antigen binding portion thereof, eliminates STAT3 phosphorylation in the cell.

In some aspects, the cell is an immune cell. In some aspects, the cell is a cancer cell.

In some aspects, the isolated antibody, or antigen binding portion thereof, inhibits or reduces inhibition of CD161 expression in a cell (e.g. ameliorates or relieves the inhibition of CD161 expression in a cell). In some aspects, the cell is an immune cell.

In some aspects, the isolated antibody, or antigen binding portion thereof, inhibits or reduces PD-L1 expression in a cell. In some aspects, PD-L1 expression is inhibited or reduced. In some aspects, TIM-3 expression is altered. In some aspects, both PD-L1 expression and TIM-3 expression is altered. In some aspects, the cell is an immune cell. In some aspects, the antibodies are monoclonal antibodies.

In some aspects, the isolated antibody, or antigen binding portion thereof, induces or enhances the PD-1-mediated secretion of one or more cytokines from a cell. In some aspects, the one or more cytokines is TNFα. In some aspects, the one or more cytokine is IL-6. In some aspects, the one or more cytokine is TNFα and IL-6. In some aspects, the cell is an immune cell.

In some aspects, the isolated antibody, or antigen binding portion thereof, is selected from the group consisting of an IgG1, an IgG2, an IgG3, an IgG4, an IgM, an IgA1 an IgA2, an IgD, and an IgE antibody. In some aspects, the antibody is an IgG1 antibody or an IgG4 antibody. In some aspects, the antibody comprises a wild type IgG1 heavy chain constant region. In some aspects, the antibody comprises a wild type IgG4 heavy chain constant region. In some aspects, the antibody comprises an Fc domain comprising at least one mutation. In some aspects, the antibody comprises a mutant IgG1 heavy chain constant region. In some aspects, the antibody comprises a mutant IgG4 heavy chain constant region. In some aspects, the mutant IgG4 heavy chain constant region comprises any one of the substitutions S228P, L235E, L235A, or a combination thereof, according to EU numbering.

In some aspects, the disclosure provides an isolated antibody, or antigen binding portion thereof, that binds to substantially the same epitope on IL-27 as the antibody, or antigen binding portion thereof, according to any one of the aforementioned aspects.

In some aspects, the disclosure provides an isolated antibody, or antigen binding portion thereof, that binds to at least one of the amino acid residues selected from the group consisting of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28) bound by the antibody, or antigen binding portion thereof, according to any one of the aforementioned aspects.

In some aspects, the disclosure provides an isolated antibody, or antigen binding portion thereof, wherein a mutation of the epitope (Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28)) bound by the antibody or antigen binding portion thereof inhibits, reduces, or blocks binding to both the antibody or antigen binding portion thereof and to the antibody or antigen binding portion thereof according to any one of the aforementioned aspects.

In some aspects, the antibody, or antigen binding portion thereof, comprises heavy chain CDR1, heavy chain CDR2, heavy chain CDR3, light chain CDR1, light chain CDR2, and light chain CDR3, wherein light chain CDR1 consists of N-XXXXXXLFSSNXKXYXX-C. In some aspects, the antibody, or antigen binding portion thereof, comprises heavy chain CDR1, heavy chain CDR2, heavy chain CDR3, light chain CDR1, light chain CDR2, and light chain CDR3, wherein light chain CDR3 consists of N-XXXASAXXX-C. In some aspects, the antibody, or antigen binding portion thereof, comprises heavy chain CDR1, heavy chain CDR2, heavy chain CDR3, light chain CDR1, light chain CDR2, and light chain CDR3, wherein heavy chain CDR2 consists of N-XXSSSXSYXYXXXXXXX-C. In some aspects, the antibody, or antigen binding portion thereof, comprises heavy chain CDR1, heavy chain CDR2, heavy chain CDR3, light chain CDR1, light chain CDR2, and light chain CDR3, wherein heavy chain CDR3 consists of N-XXXXGRTSYTATXHNXXXX-C, wherein X is any amino acids.

In some aspects, the antibody, or antigen binding portion thereof, comprises heavy chain CDR1, heavy chain CDR2, heavy chain CDR3, light chain CDR1, light chain CDR2, and light chain CDR3, wherein light chain CDR1 consists of N-XXXXXXLFSSNXKXYXX-C and light chain CDR3 consists of N-XXXASAXXX-C. In some aspects, the antibody, or antigen binding portion thereof, comprises heavy chain CDR1, heavy chain CDR2, heavy chain CDR3, light chain CDR1, light chain CDR2, and light chain CDR3, wherein heavy chain CDR2 consists of N-XXSSSXSYXYXXXXXXX-C and heavy chain CDR3 consists of N-XXXXGRTSYTATXHNXXXX-C, wherein X is any amino acids.

In some aspects, the antibody, or antigen binding portion thereof, comprises heavy chain CDR1, heavy chain CDR2, heavy chain CDR3, light chain CDR1, light chain CDR2, and light chain CDR3, wherein light chain CDR1 consists of N-XXXXXXLFSSNXKXYXX-C, light chain CDR3 consists of N-XXXASAXXX-C, heavy chain CDR2 consists of N-XXSSSXSYXYXXXXXXX-C, and heavy chain CDR3 consists of N-XXXXGRTSYTATXHNXXXX-C, wherein X is any amino acids.

In some aspects, the present disclosure provides an isolated antibody or antigen binding portion thereof that specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof does not comprise heavy and light chain CDRs selected from the group consisting of:
(i) heavy chain CDR1, CDR2 and CDR3 sequences set forth in SEQ ID NOs: 9, 10 and 11, respectively, and light chain CDR1, CDR2 and CDR3 sequences set forth in SEQ ID NOs: 17, 18 and 19, respectively;
(ii) heavy chain CDR1, CDR2 and CDR3 sequences set forth in SEQ ID NOs: 31, 32 and 33, respectively, and light chain CDR1, CDR2 and CDR3 sequences set forth in SEQ ID NOs: 39, 40 and 41, respectively;
(iii) heavy chain CDR1, CDR2 and CDR3 sequences set forth in SEQ ID NOs: 53, 54 and 55, respectively, and light chain CDR1, CDR2 and CDR3 sequences set forth in SEQ ID NOs: 61, 62 and 63, respectively;
(iv) heavy chain CDR1, CDR2 and CDR3 sequences set forth in SEQ ID NOs: 75, 76 and 77, respectively, and light chain CDR1, CDR2 and CDR3 sequences set forth in SEQ ID NOs: 83, 84 and 85, respectively;
(v) heavy chain CDR1, CDR2 and CDR3 sequences set forth in SEQ ID NOs: 97, 98 and 99, respectively, and light chain CDR1, CDR2 and CDR3 sequences set forth in SEQ ID NOs: 105, 106 and 107, respectively; or
(vi) heavy chain CDR1, CDR2 and CDR3 sequences set forth in SEQ ID NOs: 119, 120 and 121, respectively, and light chain CDR1, CDR2 and CDR3 sequences set forth in SEQ ID NOs: 127, 128 and 129, respectively.

In some aspects, the present disclosure provides an isolated antibody or antigen binding portion thereof that specifically binds to an epitope comprising or consisting of Gln37, Leu38, Glu42, Glu46, Val49, Ser50, Leu142, Asp146, Arg149, His150, Phe153, Leu156, Leu162, and Glu164 of SEQ ID NO: 2 (IL-27p28), wherein the antibody or antigen binding portion thereof does not comprise heavy and light chain CDRs selected from the group consisting of:
(i) heavy chain CDR1, CDR2 and CDR3 sequences set forth in SEQ ID NOs: 9, 10 and 11, respectively, and light chain CDR1, CDR2 and CDR3 sequences set forth in SEQ ID NOs: 17, 18 and 19, respectively;
(ii) heavy chain CDR1, CDR2 and CDR3 sequences set forth in SEQ ID NOs: 31, 32 and 33, respectively, and light chain CDR1, CDR2 and CDR3 sequences set forth in SEQ ID NOs: 39, 40 and 41, respectively;
(iii) heavy chain CDR1, CDR2 and CDR3 sequences set forth in SEQ ID NOs: 53, 54 and 55, respectively, and light chain CDR1, CDR2 and CDR3 sequences set forth in SEQ ID NOs: 61, 62 and 63, respectively;
(iv) heavy chain CDR1, CDR2 and CDR3 sequences set forth in SEQ ID NOs: 75, 76 and 77, respectively, and light chain CDR1, CDR2 and CDR3 sequences set forth in SEQ ID NOs: 83, 84 and 85, respectively;
(v) heavy chain CDR1, CDR2 and CDR3 sequences set forth in SEQ ID NOs: 97, 98 and 99, respectively, and light chain CDR1, CDR2 and CDR3 sequences set forth in SEQ ID NOs: 105, 106 and 107, respectively; or
(vi) heavy chain CDR1, CDR2 and CDR3 sequences set forth in SEQ ID NOs: 119, 120 and 121, respectively, and light chain CDR1, CDR2 and CDR3 sequences set forth in SEQ ID NOs: 127, 128 and 129, respectively.

In some aspects, the present disclosure provides an isolated antibody or antigen binding portion thereof that specifically binds to an epitope comprising or consisting of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof does not comprise heavy and light chain CDRs selected from the group consisting of:
(i) heavy chain CDR1, CDR2 and CDR3 sequences set forth in SEQ ID NOs: 9, 10 and 11, respectively, and light chain CDR1, CDR2 and CDR3 sequences set forth in SEQ ID NOs: 17, 18 and 19, respectively;
(ii) heavy chain CDR1, CDR2 and CDR3 sequences set forth in SEQ ID NOs: 31, 32 and 33, respectively, and light chain CDR1, CDR2 and CDR3 sequences set forth in SEQ ID NOs: 39, 40 and 41, respectively;
(iii) heavy chain CDR1, CDR2 and CDR3 sequences set forth in SEQ ID NOs: 53, 54 and 55, respectively, and light chain CDR1, CDR2 and CDR3 sequences set forth in SEQ ID NOs: 61, 62 and 63, respectively;
(iv) heavy chain CDR1, CDR2 and CDR3 sequences set forth in SEQ ID NOs: 75, 76 and 77, respectively, and light chain CDR1, CDR2 and CDR3 sequences set forth in SEQ ID NOs: 83, 84 and 85, respectively;
(v) heavy chain CDR1, CDR2 and CDR3 sequences set forth in SEQ ID NOs: 97, 98 and 99, respectively, and light chain CDR1, CDR2 and CDR3 sequences set forth in SEQ ID NOs: 105, 106 and 107, respectively; or
(vi) heavy chain CDR1, CDR2 and CDR3 sequences set forth in SEQ ID NOs: 119, 120 and 121, respectively, and light chain CDR1, CDR2 and CDR3 sequences set forth in SEQ ID NOs: 127, 128 and 129, respectively.

In some aspects, the present disclosure provides an isolated antibody or antigen binding portion thereof that specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof does not comprise heavy and light chain CDRs selected from the group consisting of:
(i) heavy chain CDR1, CDR2 and CDR3 sequences set forth in SEQ ID NOs: 12, 13 and 14, respectively, and light chain CDR1, CDR2 and CDR3 sequences set forth in SEQ ID NOs: 20, 21 and 22, respectively;
(ii) heavy chain CDR1, CDR2 and CDR3 sequences set forth in SEQ ID NOs: 34, 35 and 36, respectively, and light chain CDR1, CDR2 and CDR3 sequences set forth in SEQ ID NOs: 42, 43 and 44, respectively;
(iii) heavy chain CDR1, CDR2 and CDR3 sequences set forth in SEQ ID NOs: 56, 57 and 58, respectively, and light chain CDR1, CDR2 and CDR3 sequences set forth in SEQ ID NOs: 64, 65 and 66, respectively;
(iv) heavy chain CDR1, CDR2 and CDR3 sequences set forth in SEQ ID NOs: 78, 79 and 80, respectively, and light chain CDR1, CDR2 and CDR3 sequences set forth in SEQ ID NOs: 86, 88 and 89, respectively;
(v) heavy chain CDR1, CDR2 and CDR3 sequences set forth in SEQ ID NOs: 100, 101 and 102, respectively, and light chain CDR1, CDR2 and CDR3 sequences set forth in SEQ ID NOs: 108, 109 and 110, respectively; or
(vi) heavy chain CDR1, CDR2 and CDR3 sequences set forth in SEQ ID NOs: 122, 123 and 124, respectively, and light chain CDR1, CDR2 and CDR3 sequences set forth in SEQ ID NOs: 130, 131 and 132, respectively.

In some aspects, the present disclosure provides an isolated antibody or antigen binding portion thereof that specifically binds to an epitope comprising or consisting of Gln37, Leu38, Glu42, Glu46, Val49, Ser50, Leu142, Asp146, Arg149, His 150, Phe153, Leu156, Leu162, and Glu164 of SEQ ID NO: 2 (IL-27p28), wherein the antibody or antigen binding portion thereof does not comprise heavy and light chain CDRs selected from the group consisting of:
(i) heavy chain CDR1, CDR2 and CDR3 sequences set forth in SEQ ID NOs: 12, 13 and 14, respectively, and light chain CDR1, CDR2 and CDR3 sequences set forth in SEQ ID NOs: 20, 21 and 22, respectively;
(ii) heavy chain CDR1, CDR2 and CDR3 sequences set forth in SEQ ID NOs: 34, 35 and 36, respectively, and light chain CDR1, CDR2 and CDR3 sequences set forth in SEQ ID NOs: 42, 43 and 44, respectively;
(iii) heavy chain CDR1, CDR2 and CDR3 sequences set forth in SEQ ID NOs: 56, 57 and 58, respectively, and light chain CDR1, CDR2 and CDR3 sequences set forth in SEQ ID NOs: 64, 65 and 66, respectively;
(iv) heavy chain CDR1, CDR2 and CDR3 sequences set forth in SEQ ID NOs: 78, 79 and 80, respectively, and light chain CDR1, CDR2 and CDR3 sequences set forth in SEQ ID NOs: 86, 88 and 89, respectively;
(v) heavy chain CDR1, CDR2 and CDR3 sequences set forth in SEQ ID NOs: 100, 101 and 102, respectively, and light chain CDR1, CDR2 and CDR3 sequences set forth in SEQ ID NOs: 108, 109 and 110, respectively; or
(vi) heavy chain CDR1, CDR2 and CDR3 sequences set forth in SEQ ID NOs: 122, 123 and 124, respectively, and light chain CDR1, CDR2 and CDR3 sequences set forth in SEQ ID NOs: 130, 131 and 132, respectively.

In some aspects, the present disclosure provides an isolated antibody or antigen binding portion thereof that specifically binds to an epitope comprising or consisting of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof does not comprise heavy and light chain CDRs selected from the group consisting of:
(i) heavy chain CDR1, CDR2 and CDR3 sequences set forth in SEQ ID NOs: 12, 13 and 14, respectively, and light chain CDR1, CDR2 and CDR3 sequences set forth in SEQ ID NOS: 20, 21 and 22, respectively;
(ii) heavy chain CDR1, CDR2 and CDR3 sequences set forth in SEQ ID NOs: 34, 35 and 36, respectively, and light chain CDR1, CDR2 and CDR3 sequences set forth in SEQ ID NOs: 42, 43 and 44, respectively;
(iii) heavy chain CDR1, CDR2 and CDR3 sequences set forth in SEQ ID NOs: 56, 57 and 58, respectively, and light chain CDR1, CDR2 and CDR3 sequences set forth in SEQ ID NOs: 64, 65 and 66, respectively;
(iv) heavy chain CDR1, CDR2 and CDR3 sequences set forth in SEQ ID NOs: 78, 79 and 80, respectively, and light chain CDR1, CDR2 and CDR3 sequences set forth in SEQ ID NOS: 86, 88 and 89, respectively;
(v) heavy chain CDR1, CDR2 and CDR3 sequences set forth in SEQ ID NOs: 100, 101 and 102, respectively, and light chain CDR1, CDR2 and CDR3 sequences set forth in SEQ ID NOs: 108, 109 and 110, respectively; or
(vi) heavy chain CDR1, CDR2 and CDR3 sequences set forth in SEQ ID NOs: 122, 123 and 124, respectively, and light chain CDR1, CDR2 and CDR3 sequences set forth in SEQ ID NOs: 130, 131 and 132, respectively.

In some aspects, the present disclosure provides an isolated antibody or antigen binding portion thereof that specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy chain CDR1, a heavy chain CDR2, a heavy chain CDR3, a light chain CDR1, a light chain CDR2, and a light chain CDR3 and wherein the heavy chain CDR1 does not consist of N-GFTF[S/A/R][S/R][T/Y][G/S]-C (SEQ ID NO: 144) and/or the heavy chain CDR2 does not consist of N-ISSS[S/G][S/A]YI-C (SEQ ID NO: 146).

In some aspects, the present disclosure provides an isolated antibody or antigen binding portion thereof that specifically binds to an epitope comprising or consisting of Gln37, Leu38, Glu42, Glu46, Val49, Ser50, Leu142, Asp146, Arg149, His150, Phe153, Leu156, Leu162, and Glu164 of SEQ ID NO: 2 (IL-27p28), wherein the antibody or antigen binding portion thereof comprises a heavy chain CDR1, a heavy chain CDR2, a heavy chain CDR3, a light chain CDR1, a light chain CDR2, and a light chain CDR3 and wherein the heavy chain CDR1 does not consist of N-GFTF[S/A/R][S/R][T/Y][G/S]-C (SEQ ID NO: 144) and/or the heavy chain CDR2 does not consist of N-ISSS[S/G][S/A]YI-C (SEQ ID NO: 146).

In some aspects, the present disclosure provides an isolated antibody or antigen binding portion thereof that specifically binds to an epitope comprising or consisting of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy chain CDR1, a heavy chain CDR2, a heavy chain CDR3, a light chain CDR1, a light chain CDR2, and a light chain CDR3 and wherein the heavy chain CDR1 does not consist of N-GFTF[S/A/R][S/R][T/Y][G/S]-C (SEQ ID NO: 144) and/or the heavy chain CDR2 does not consist of N-ISSS[S/G][S/A]YI-C (SEQ ID NO: 146).

In some aspects, the present disclosure provides an isolated antibody or antigen binding portion thereof that specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy chain CDR1, a heavy chain CDR2, a heavy chain CDR3, a light chain CDR1, a light chain CDR2, and a light chain CDR3 and wherein the heavy chain CDR1 does not comprise N-FTF[S/A/R][S/R][T/Y][G/S]MN-C (SEQ ID NO: 148) and/or the heavy chain CDR2 does not comprise N-[G/S]ISSS[S/G][S/A]YI[L/Y]YADSVKG-C (SEQ ID NO: 149).

In some aspects, the present disclosure provides an isolated antibody or antigen binding portion thereof that specifically binds to an epitope comprising or consisting of Gln37, Leu38, Glu42, Glu46, Val49, Ser50, Leu142, Asp146, Arg149, His 150, Phe153, Leu156, Leu162, and Glu164 of SEQ ID NO: 2 (IL-27p28), wherein the antibody or antigen binding portion thereof comprises a heavy chain CDR1, a heavy chain CDR2, a heavy chain CDR3, a light chain CDR1, a light chain CDR2, and a light chain CDR3 and wherein the heavy chain CDR1 does not comprise N-FTF[S/A/R][S/R][T/Y][G/S]MN-C (SEQ ID NO: 148) and/or the heavy chain CDR2 does not comprise N-[G/S]ISSS[S/G][S/A]YI[L/Y]YADSVKG-C (SEQ ID NO: 149).

In some aspects, the present disclosure provides an isolated antibody or antigen binding portion thereof that specifically binds to an epitope comprising or consisting of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy chain CDR1, a heavy chain CDR2, a heavy chain CDR3, a light chain CDR1, a light chain CDR2, and a light chain CDR3 and wherein the heavy chain CDR1 does not comprise N-FTF[S/A/R][S/R][T/Y][G/S]MN-C (SEQ ID NO: 148) and/or the heavy chain CDR2 does not comprise N-[G/S]ISSS[S/G][S/A]YI[L/Y]YADSVKG-C (SEQ ID NO: 149).

In some aspects, the present disclosure provides an isolated antibody or antigen binding portion thereof that specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof does not comprise:
(i) heavy chain CDR1 consisting of N-GFTFXXXX-C (SEQ ID NO: 145), heavy chain CDR2 consisting of N-ISSSXXYI-C (SEQ ID NO: 147), and heavy chain CDR3 sequence set forth in SEQ ID NO: 121; and light chain CDR1, CDR2 and CDR3 sequences set forth in SEQ ID NOs: 127, 128 and 129, respectively; or
(ii) heavy chain CDR1 consisting of N-FTFXXXXMN-C (SEQ ID NO: 150), heavy chain CDR2 consisting of N-XISSSXXYIXYADSVKG-C (SEQ ID NO: 151), and heavy chain CDR3 sequence set forth in SEQ ID NO: 124; and light chain CDR1, CDR2 and CDR3 sequences set forth in SEQ ID NOs: 130, 131 and 132, respectively.

In some aspects, the present disclosure provides an isolated antibody or antigen binding portion thereof that specifically binds to an epitope comprising or consisting of Gln37, Leu38, Glu42, Glu46, Val49, Ser50, Leu142, Asp146, Arg149, His 150, Phe153, Leu156, Leu162, and Glu164 of SEQ ID NO: 2 (IL-27p28), wherein the antibody or antigen binding portion thereof does not comprise:
(i) heavy chain CDR1 consisting of N-GFTFXXXX-C (SEQ ID NO: 145), heavy chain CDR2 consisting of N-ISSSXXYI-C (SEQ ID NO: 147), and heavy chain CDR3 sequence set forth in SEQ ID NO: 121; and light chain CDR1, CDR2 and CDR3 sequences set forth in SEQ ID NOs: 127, 128 and 129, respectively; or
(ii) heavy chain CDR1 consisting of N-FTFXXXXMN-C (SEQ ID NO: 150), heavy chain CDR2 consisting of N-XISSSXXYIXYADSVKG-C (SEQ ID NO: 151), and heavy chain CDR3 sequence set forth in SEQ ID NO: 124; and light chain CDR1, CDR2 and CDR3 sequences set forth in SEQ ID NOs: 130, 131 and 132, respectively.

In some aspects, the present disclosure provides an isolated antibody or antigen binding portion thereof that specifically binds to an epitope comprising or consisting of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof does not comprise:
(i) heavy chain CDR1 consisting of N-GFTFXXXX-C (SEQ ID NO: 145), heavy chain CDR2 consisting of N-ISSSXXYI-C (SEQ ID NO: 147), and heavy chain CDR3 sequence set forth in SEQ ID NO: 121; and light chain CDR1, CDR2 and CDR3 sequences set forth in SEQ ID NOs: 127, 128 and 129, respectively; or
(ii) heavy chain CDR1 consisting of N-FTFXXXXMN-C (SEQ ID NO: 150), heavy chain CDR2 consisting of N-XISSSXXYIXYADSVKG-C (SEQ ID NO: 151), and heavy chain CDR3 sequence set forth in SEQ ID NO: 124; and light chain CDR1, CDR2 and CDR3 sequences set forth in SEQ ID NOs: 130, 131 and 132, respectively.

In some aspects, the present disclosure provides an isolated antibody or antigen binding portion thereof that specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof does not comprise: heavy chain CDR1 consisting of N-GFTFXXXX-C (SEQ ID NO: 145), heavy chain CDR2 consisting of N-IXXXXXXX-C (SEQ ID NO: 152), and heavy chain CDR3 sequence consisting of N-AR[X]ₙ₌₆₋₁₅DX-C (SEQ ID NO: 153); and light chain CDR1 consisting of N-QS[X]ₙ₌₁₋₃SS[X]ₙ₌₀₋₄Y-C (SEQ ID NO: 154), light chain CDR2 consisting of N-XXS-C(SEQ ID NO: 155), and light chain CDR3 sequence consisting of N-QQXXXXP [X]ₙ₌₀₋₁T-C (SEQ ID NO: 156), respectively.

In some aspects, the present disclosure provides an isolated antibody or antigen binding portion thereof that specifically binds to an epitope comprising or consisting of Gln37, Leu38, Glu42, Glu46, Val49, Ser50, Leu142, Asp146, Arg149, His150, Phe153, Leu156, Leu162, and Glu164 of SEQ ID NO: 2 (IL-27p28), wherein the antibody or antigen binding portion thereof does not comprise: heavy chain CDR1 consisting of N-GFTFXXXX-C (SEQ ID NO: 145), heavy chain CDR2 consisting of N-IXXXXXXX-C (SEQ ID NO: 152), and heavy chain CDR3 sequence consisting of N-AR[X]ₙ₌₆₋₁₅DX-C (SEQ ID NO: 153); and light chain CDR1 consisting of N-QS[X]ₙ₌₁₋₃SS[X]ₙ₌₀₋₄Y-C (SEQ ID NO: 154), light chain CDR2 consisting of N-XXS-C (SEQ ID NO: 155), and light chain CDR3 sequence consisting of N-QQXXXXP [X]ₙ₌₀₋₁T-C (SEQ ID NO: 156), respectively.

In some aspects, the present disclosure provides an isolated antibody or antigen binding portion thereof that specifically binds to an epitope comprising or consisting of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof does not comprise: heavy chain CDR1 consisting of N-GFTFXXXX-C (SEQ ID NO: 145), heavy chain CDR2 consisting of N-IXXXXXXX-C (SEQ ID NO: 152), and heavy chain CDR3 sequence consisting of N-AR[X]ₙ₌₆₋₁₅DX-C (SEQ ID NO: 153); and light chain CDR1 consisting of N-QS[X]ₙ₌₁₋₃SS[X]ₙ₌₀₋₄Y-C (SEQ ID NO: 154), light chain CDR2 consisting of N-XXS-C (SEQ ID NO: 155), and light chain CDR3 sequence consisting of N-QQXXXXP[X]ₙ₌₀₋₁T-C (SEQ ID NO: 156), respectively.

In some aspects, the present disclosure provides an isolated antibody or antigen binding portion thereof that antagonizes IL-27 and specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises heavy and light chain variable regions, wherein the heavy chain variable region does not comprise an amino acid sequence selected from the group consisting of SEQ ID NOs: 15, 37, 59, 81, 103, and 125; and wherein the light chain variable region does not comprise an amino acid sequence selected from the group consisting of SEQ ID NOs: 23, 45, 67, 89, 111, and 133.

In some aspects, the present disclosure provides an isolated antibody or antigen binding portion thereof that antagonizes IL-27 and specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises heavy and light chain variable regions, wherein the heavy chain variable region and the light chain variable region are not amino acid sequences selected from the group consisting of:
(i) SEQ ID NO: 15 and 65, respectively;
(ii) SEQ ID NO: 37 and 45, respectively;
(iii) SEQ ID NO: 59 and 67, respectively;
(iv) SEQ ID NO: 81 and 89, respectively;
(v) SEQ ID NO: 103 and 111, respectively; and
(vi) SEQ ID NO: 125 and 133, respectively.

In some aspects, the present disclosure provides an isolated antibody or antigen binding portion thereof that antagonizes IL-27 and specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises heavy and light chain variable regions, wherein the heavy chain variable region does not comprise an amino acid sequence which is at least 90% identical to the amino acid sequence selected from the group consisting of SEQ ID NOs: 15, 37, 59, 81, 103, and 125; and wherein the light chain variable region does not comprise an amino acid sequence which is at least 90% identical to the amino acid sequence selected from the group consisting of SEQ ID NOs: 23, 45, 67, 89, 111, and 133.

In some aspects, the present disclosure provides an isolated antibody or antigen binding portion thereof that antagonizes IL-27 and specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises heavy and light chain variable regions, wherein the heavy chain variable region and the light chain variable region do not comprise amino acid sequences at least 90% identical to the amino acid sequences selected from the group consisting of:
(i) SEQ ID NO: 15 and 65, respectively;
(ii) SEQ ID NO: 37 and 45, respectively;
(iii) SEQ ID NO: 59 and 67, respectively;
(iv) SEQ ID NO: 81 and 89, respectively;
(v) SEQ ID NO: 103 and 111, respectively; and
(vi) SEQ ID NO: 125 and 133, respectively.

In some aspects, the present disclosure provides an isolated antibody or antigen binding portion thereof that antagonizes IL-27 and specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy chain and a light chain, wherein the heavy chain does not comprise an amino acid sequence selected from the group consisting of SEQ ID NOs: 25,47, 69, 91, 113, and 135; and wherein the light chain does not comprise an amino acid sequence selected from the group consisting of SEQ ID NOs: 20, 42, 71, 93, and 1115.

In some aspects, the present disclosure provides an isolated antibody or antigen binding portion thereof that antagonizes IL-27 and specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy and a light chain, wherein the heavy chain does not comprise an amino acid sequence which is at least 90% identical to the amino acid sequence selected from the group consisting of SEQ ID NOs: 25,47, 69, 91, 113, and 135; and wherein the light chain does not comprise an amino acid sequence which is at least 90% identical to the amino acid sequence selected from the group consisting of SEQ ID NOs: 20, 42, 71, 93, and 115.

In some aspects, the present disclosure provides an isolated antibody or antigen binding portion thereof that antagonizes IL-27 and specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy chain and a light chain, wherein the heavy chain does not comprise an amino acid sequence selected from the group consisting of SEQ ID NOs: 29, 51, 73, 95, 117, and 139; and wherein the light chain does not comprise an amino acid sequence selected from the group consisting of SEQ ID NOs: 71, 49, 71, 93, 115, and 137.

In some aspects, the present disclosure provides an isolated antibody or antigen binding portion thereof that antagonizes IL-27 and specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy and a light chain, wherein the heavy chain does not comprise an amino acid sequence which is at least 90% identical to the amino acid sequence selected from the group consisting of SEQ ID NOs: 29, 51, 73, 95, 117, and 139; and wherein the light chain does not comprise an amino acid sequence which is at least 90% identical to the amino acid sequence selected from the group consisting of SEQ ID NOs: 71, 49, 71, 93, 115, and 137.

In some aspects, the present disclosure provides an isolated antibody or antigen binding portion thereof that antagonizes IL-27 and specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy chain and a light chain, and wherein the heavy chain and the light chain do not comprise amino acid sequences selected from the group consisting of:
(i) SEQ ID NO: 25 and 27, respectively;
(ii) SEQ ID NO: 47 and 49, respectively;
(iii) SEQ ID NO: 69 and 71, respectively;
(iv) SEQ ID NO: 91 and 93, respectively;
(v) SEQ ID NO: 113 and 115, respectively; and
(vi) SEQ ID NO: 135 and 137, respectively.

In some aspects, the present disclosure provides an isolated antibody or antigen binding portion thereof that antagonizes IL-27 and specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy chain and a light chain and wherein the heavy chain and the light chain do not comprise amino acid sequences at least 90% identical to the amino acid sequences selected from the group consisting of:
(i) SEQ ID NO: 25 and 27, respectively;
(ii) SEQ ID NO: 47 and 49, respectively;
(iii) SEQ ID NO: 69 and 71, respectively;
(iv) SEQ ID NO: 91 and 93, respectively;
(v) SEQ ID NO: 113 and 115, respectively; and
(vi) SEQ ID NO: 135 and 137, respectively.

In some aspects, the present disclosure provides an isolated antibody or antigen binding portion thereof that antagonizes IL-27 and specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy and a light chain and wherein the heavy chain and the light chain do not comprise amino acid sequences selected from the group consisting of:
(i) SEQ ID NO: 29 and 27, respectively;
(ii) SEQ ID NO: 51 and 49, respectively;
(iii) SEQ ID NO: 73 and 72, respectively;
(iv) SEQ ID NO: 95 and 93, respectively;
(v) SEQ ID NO: 117 and 115, respectively; and
(vi) SEQ ID NO: 139 and 137, respectively.

In some aspects, the present disclosure provides an isolated antibody or antigen binding portion thereof that antagonizes IL-27 and specifically binds to an epitope comprising one or more amino acids of Gln37, Leu38, Glu42, Glu46, Val49, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL27-p28), wherein the antibody or antigen binding portion thereof comprises a heavy and a light chain and wherein the heavy chain and the light chain do not comprise amino acid sequences at least 90% identical to the amino acid sequences selected from the group consisting of: (i) SEQ ID NO: 29 and 27, respectively; (ii) SEQ ID NO: 51 and 49, respectively; (iii) SEQ ID NO: 73 and 72, respectively; (iv) SEQ ID NO: 95 and 93, respectively; (v) SEQ ID NO: 117 and 115, respectively; and (vi) SEQ ID NO: 139 and 137, respectively.

### D. Combination Therapy

In some aspects, an IL-27-inhibiting agent, e.g., an anti-IL-27 antibody, or antigen binding portion thereof, provided by the disclosure, can be combined with one or more additional therapeutics or treatments, e.g., another therapeutic or treatment for a cancer. For example, the IL-27-inhibiting agent, e.g., anti-IL-27 antibody, or antigen binding portion thereof, can be administered to a subject (e.g., a human patient) in combination with one or more additional therapeutics, wherein the combination provides a therapeutic benefit to a subject who has, or is at risk of developing, cancer.

In some aspects, an IL-27-inhibiting agent, e.g., anti-IL-27 antibody, or antigen binding portion thereof, and the one or more additional therapeutics are administered at the same time (e.g., simultaneously). In other aspects, the IL-27-inhibiting agent, e.g., anti-IL-27 antibody, or antigen binding portion thereof, is administered first in time and the one or more additional therapeutics are administered second in time (e.g., sequentially). In some aspects, the one or more additional therapeutics are administered first in time and the IL-27-inhibiting agent, e.g., anti-IL-27 antibody, is administered second in time.

An IL-27-inhibiting agent, e.g., anti-IL-27 antibody or an antigen-binding fragment thereof described herein can replace or augment a previously or currently administered therapy. For example, upon treating with an anti-IL-27 antibody or antigen-binding fragment thereof, administration of the one or more additional therapeutics can cease or diminish, e.g., be administered at lower levels. In some aspects, administration of the previous therapy can be maintained. In some aspects, a previous therapy will be maintained until the level of the anti-IL-27 antibody reaches a level sufficient to provide a therapeutic effect.

In some aspects, the disclosure provides a method of treating cancer in a subject, the method comprising administering to the subject an effective amount of an isolated antibody, or antigen binding portion thereof, that specifically binds to and antagonizes IL-27, provided by the disclosure, in combination with one or more additional therapeutic agents or procedure, wherein the second therapeutic agent or procedure is selected from the group consisting of: a chemotherapy, a targeted anti-cancer therapy, an oncolytic drug, a cytotoxic agent, an immune-based therapy, a cytokine, surgical procedure, a radiation procedure, an activator of a costimulatory molecule, an inhibitor of an inhibitory molecule, a vaccine, or a cellular immunotherapy, a biologic agent, or a combination thereof.

In some aspects, the one or more additional therapeutic agents is a PD-1 antagonist, a TIM-3 inhibitor, a LAG-3 inhibitor, a TIGIT inhibitor, a CD112R inhibitor, a TAM inhibitor, a STING agonist, a 4-1BB agonist, or a combination thereof. In some aspects, the one or more additional therapeutic agents is a CD39 antagonist, a CD73 antagonist, a CCR8 antagonist, or a combination thereof. In some aspects, the anti-CD73 is any anti-CD73 antibody disclosed in, e.g., U.S. Publication No. 2019/0031766 A1, which is incorporated by reference herein in its entirety. In some aspects, the anti-CD39 is any anti-CD39 antibody disclosed in, e.g., Int'l Publication No. WO 2019/178269 A2, which is incorporated by reference herein in its entirety.

In some aspects, the one or more additional therapeutic agents is a PD-1 antagonist. In some aspects, the PD-1 antagonist is selected from the group consisting of: PDR001, nivolumab, pembrolizumab, pidilizumab, tislelizumab, zimberelimuab, MEDI0680, REGN2810, TSR-042, PF-06801591, and AMP-224. In certain aspects, the one or more additional therapeutic agents is a PD-L1 inhibitor. In some aspects, the PD-L1 inhibitor is selected from the group consisting of: FAZ053, Atezolizumab, Avelumab, Durvalumab, and BMS-936559. In some aspects, the disclosure provides a method of enhancing one or more activities of an anti-PD-1 antibody (e.g., enhances PD-1-mediated cytokine secretion; enhances anti-PD-1 mediated TNFα secretion; enhances anti-PD-1 mediated IL-6 secretion from a cell exposed to anti-PD-1 antibodies), the method comprising exposing a cell to an antibody, or antigen binding portion thereof, provided by the disclosure, concurrently with or sequentially to an anti-PD-1 antibody, thereby to enhance one or more activities of the anti-PD1 antibody.

In some aspects, the one or more additional therapeutic agents is Sunitinib (Sutent®), Cabozantinib (CABOMETYX®), Axitinib (INLYTA®), Lenvatinib (LENVIMA®), Everolimus (AFINITOR®), Bevacizumab (AVASTIN®), epacadostat, NKTR-214 (CD-122-biased agonist), tivozanib (FOTIVDA®), abexinostat, Ipilimumab (YERVOY®), tremelimumab, Pazopanib (VOTRIENT®), Sorafenib (NEXAVAR®), Temsirolimus (TORISEL®), Ramucirumab (CYRAMZA®), niraparib, savolitinib, vorolanib (X-82), Regorafenib (STIVARGO®), Donafenib (multikinase inhibitor), Camrelizumab (SHR-1210), pexastimogene devacirepvec (JX-594), Ramucirumab (CYRAMZA®), (YN968D1), apatinib encapsulated doxorubicin (THERMODOX®), Tivantinib (ARQ197), ADI-PEG 20, binimetinib, apatinib mesylate, nintedanib, lirilumab, Nivolumab (OPDIVO®), Pembrolizumab (KEYTRUDA®), Atezolizumab (TECENTRIQ®), Avelumab (BAVENCIO®), Durvalumab (IMFIMZI®), Cemiplimab-rwlc (LIBTAYO®), tislelizumab, and/or spartalizumab.

In some aspects, the one or more additional therapeutic agents is a TIM-3 inhibitor, optionally wherein the TIM-3 inhibitor is MGB453 or TSR-022.

In some aspects, the one or more additional therapeutic agents is a LAG-3 inhibitor, optionally wherein the LAG-3 inhibitor is selected from the group consisting of LAG525, BMS-986016, and TSR-033.

In some aspects, the one or more additional therapeutic agents is a TIGIT inhibitor. In some aspects, the one or more additional therapeutic agents is a CD112R inhibitor. In some aspects, the one or more additional therapeutic agents is a TAM (Axl, Mer, Tyro) inhibitor. In some aspects, the one or more additional therapeutic agents is a STING agonist. In some aspects, the one or more additional therapeutic agents is a 4-1BB agonist.

In some aspects, the one or more additional therapeutic agents is a tyrosine kinase inhibitor, an agent targeting the adenosine axis (for example a CD39 antagonist, a CD73 antagonist or a A2AR, A2BR or dual A2AR/A2BR antagonist), a CCR8 antagonist, a CTLA4 antagonist, a VEG-F inhibitor or a combination thereof.

1. Combination with Chemotherapeutic Agents

In some aspects, the methods disclosed herein comprise administering an IL-27-inhibiting agent, e.g., an antibody or an antigen-binding portion thereof that specifically binds to to IL-27, and a chemotherapeutic agent. Chemotherapeutic agents suitable for combination and/or co-administration with compositions of the present disclosure include, for example: taxol, cytochalasin B, gramicidin D, ethidium bromide, emetine, mitomycin, etoposide, tenoposide, vincristine, vinblastine, colchicin, doxorubicin, daunorubicin, dihydroxyanthrancindione, mitoxantrone, mithramycin, actinomycin D, 1-dehydrotestosterone, glucocorticoids, procaine, tetracaine, lidocaine, propranolol, and puromycin and analogs or homologs thereof. Further agents include, for example, antimetabolites (e.g., methotrexate, 6-mercaptopurine, 6-thioguanine, cytarabine, 5-fluorouracil decarbazine), alkylating agents (e.g., mechlorethamine, thioTEPA, chlorambucil, melphalan, carmustine (BSNU), lomustine (CCNU), cyclophosphamide, busulfan, dibromomannitol, streptozotocin, mitomycin C, cis-dichlordiamine platinum (II) (DDP), procarbazine, altretamine, cisplatin, carboplatin, oxaliplatin, nedaplatin, satraplatin, or triplatin tetranitrate), anthracycline (e.g., daunorubicin (formerly daunomycin) and doxorubicin), antibiotics (e.g., dactinomcin (formerly actinomycin), bleomycin, mithramycin, and anthramycin (AMC)), and anti-mitotic agents (e.g., vincristine and vinblastine) and temozolomide.

2. Combination with PD-1/PD-L1 Antagonists

In some aspects, the methods disclosed herein comprise administering an IL-27-inhibiting agent, e.g., an antibody or an antigen-binding portion thereof that specifically binds to to IL-27, and one or more PD-1 antagonist. In some aspects, the one or more PD-1 antagonist specifically binds to human PD-1 or PD-L1 and inhibits PD-1/PD-L1 biological activity and/or downstream pathway(s) and/or cellular processed mediated by human PD-1/PD-L1 signaling or other human PD-1/PD-L1-mediated functions.

Accordingly, provided herein are PD-1 antagonists that directly or allosterically block, antagonize, suppress, inhibit or reduce PD-1/PD-L1 biological activity, including downstream pathways and/or cellular processes mediated by PD-1/PD-L1 signaling, such as receptor binding and/or elicitation of a cellular response to PD-1/PD-L1. Also provided herein are PD-1 antagonists that reduce the quantity or amount of human PD-1 or PD-L1 produced by a cell or subject.

In some aspects, the disclosure provides a PD-1 antagonist that binds human PD-1 and prevents, inhibits or reduces PD-L1 binding to PD-1. In some aspects, the PD-1 antagonist binds to the mRNA encoding PD-1 or PD-L1 and prevents translation. In some aspects, the PD-1 antagonist binds to the mRNA encoding PD-1 or PD-L1 and causes degradation and/or turnover.

In some aspects, the PD-1 antagonist inhibits PD-1 signaling or function. In some aspects, the PD-1 antagonist blocks binding of PD-1 to PD-L1, PD-L2, or to both PD-L1 and PD-L2. In some aspects, the PD-1 antagonist blocks binding of PD-1 to PD-L1. In some aspects, the PD-1 antagonist blocks binding of PD-1 to PD-L2. In some aspects, the PD-1 antagonist blocks the binding of PD-1 to PD-L1 and PD-L2. In some aspects, the PD-1 antagonist specifically binds PD-1. In some aspects, the PD-1 antagonist specifically binds PD-L1. In some aspects, the PD-1 antagonist specifically binds PD-L2.

In some aspects, the PD-1 antagonist inhibits the binding of PD-1 to its cognate ligand. In some aspects, the PD-1 antagonist inhibits the binding of PD-1 to PD-L1, PD-1 to PD-L2, or PD-1 to both PD-L1 and PD-L2. In some aspects, the PD-1 antagonist does not inhibit the binding of PD-1 to its cognate ligand.

In some aspects, the PD-1 antagonist is an isolated antibody (mAb), or antigen binding fragment thereof, which specifically binds to PD-1 or PD-L1. In some aspects, the PD-1 antagonist is an antibody or antigen binding fragment thereof that specifically binds to human PD-1. In some aspects, the PD-1 antagonist is an antibody or antigen binding fragment thereof that specifically binds to human PD-L1. In some aspects, the PD-1 antagonist is an antibody or antigen binding fragment that binds to human PD-L1 and inhibits the binding of PD-L1 to PD-1. In some aspects, the PD-1 antagonist is an antibody or antigen binding fragment that binds to human PD-1 and inhibits the binding of PD-L1 to PD-1.

Several immune checkpoint antagonists that inhibit or disrupt the interaction between PD-1 and either one or both of its ligands PD-L1 and PD-L2 are in clinical development or are currently available to clinicians for treating cancer.

Examples of anti-human PD-1 antibodies, or antigen binding fragments thereof, that may comprise the PD-1 antagonist in any of the compositions, methods, and uses provided by the disclosure include, but are not limited to: KEYTRUDA® (pembrolizumab, MK-3475, h409A11; see U.S. Pat. Nos. 8,952,136, 8,354,509, 8,900,587, and EP2170959, all of which are included herein by reference in their entirety; Merck), OPDIVO® (nivolumab, BMS-936558, MDX-1106, ONO-4538; see U.S. Pat. Nos. 7,595,048, 8,728,474, 9,073,994, 9,067,999, EP1537878, U.S. Pat. Nos. 8,008,449, 8,779,105, and EP2161336, all of which are included herein by reference in their entirety; Bristol Myers Squibb), MEDI0680 (AMP-514), BGB-A317 and BGB-108 (BeiGene), 244C8 and 388D4 (see WO2016106159, which is incorporated herein by reference in its entirety; Enumeral Biomedical), PDR001 (Novartis), and REGN2810 (Regeneron). Accordingly, in some aspects the PD-1 antagonist is pembrolizumab. In some aspects, the PD-1 antagonist is nivolumab. In some aspects, the methods disclosed herein comprise administering an antibody or an antigen-binding portion thereof that specifically binds to to IL-27 and pembrolizumab. In some aspects, the methods disclosed herein comprise administering an antibody or an antigen-binding portion thereof that specifically binds to to IL-27 and nivolumab.

Examples of anti-human PD-L1 antibodies, or antigen binding fragments thereof, that may comprise the PD-1 antagonist in any of the compositions, methods, and uses provided by the disclosure include, but are not limited to: BAVENCIO® (avelumab, MSB0010718C, see WO2013/79174, which is incorporated herein by reference in its entirety; Merck/Pfizer), IMFINZI® (durvalumab, MEDI4736), TECENTRIQ® (atezolizumab, MPDL3280A, RG7446; see WO2010/077634, which is incorporated herein by reference in its entirety; Roche), MDX-1105 (BMS-936559, 12A4; see U.S. Pat. No. 7,943,743 and WO2013/173223, both of which are incorporated herein by reference in their entirety; Medarex/BMS), and FAZ053 (Novartis). Accordingly, in some aspects the PD-1 antagonist is avelumab. In some aspects, the PD-1 antagonist is durvalumab. In some aspects, the PD-1 antagonist is atezolizumab.

In some aspects, the PD-1 antagonist is an immunoadhesin that specifically bind to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342, both of which are incorporated herein by reference in their entirety. In some aspects, the PD-1 antagonist is AMP-224 (also known as B7-DCIg), which is a PD-L2-FC fusion protein that specifically binds to human PD-1.

It will be understood by one of ordinary skill that any PD-1 antagonist which binds to PD-1 or PD-L1 and disrupts the PD-1/PD-L1 signaling pathway, is suitable for compositions, methods, and uses disclosed herein.

In some aspects, the PD-1/PD-L1 antagonist is a small molecule, a nucleic acid, a peptide, a peptide mimetic, a protein, a carbohydrate, a carbohydrate derivative, or a glycopolymer. Exemplary small molecule PD-1 inhibitors are described in Zhan et al., (2016) Drug Discov Today 21 (6): 1027-1036.

3. Combinations with TIM-3 Inhibitors

In some aspects, the methods disclosed herein comprise administering an IL-27-inhibiting agent, e.g., an antibody or an antigen-binding portion thereof that specifically binds to to IL-27 and a TIM-3 inhibitor. The TIM-3 inhibitor may be an antibody, an antigen binding fragment thereof, an immunoadhesin, a fusion protein, or an oligopeptide. In some aspects, the TIM-3 inhibitor is chosen from MGB453 (Novartis), TSR-022 (Tesaro), or LY3321367 (Eli Lilly). In some aspects, the anti-IL-27 antibody, or antigen binding portion thereof, is administered in combination with MGB453. In some aspects, the anti-IL-27 antibody, or antigen binding portion thereof, is administered in combination with TSR-022.

4. Combinations with LAG-3 Inhibitors

In some aspects, the methods disclosed herein comprise administering an IL-27-inhibiting agent, e.g., an antibody or an antigen-binding portion thereof that specifically binds to to IL-27, and a LAG-3 inhibitor. In some aspects, the LAG-3 inhibitor is an antibody, an antigen binding fragment thereof, an immunoadhesin, a fusion protein, an oligopeptide, or any combination thereof. In some aspects, the LAG-3 inhibitor is chosen from LAG525 (Novartis), BMS-986016 (Bristol-Myers Squibb), TSR-033 (Tesaro), MK-4280 (Merck & Co), or REGN3767 (Regeneron).

### 5. Other Combinations

In some aspects, the methods disclosed herein comprise administering an IL-27-inhibiting agent, e.g., an antibody or an antigen-binding portion thereof that specifically binds to to IL-27, and a TIGIT inhibitor. In some aspects, the methods disclosed herein comprise administering an IL-27-inhibiting agent, e.g., an antibody or an antigen-binding portion thereof that specifically binds to to IL-27, and a kinase inhibitor (e.g., a tyrosine kinase inhibitor (TKI)). In some aspects, the methods disclosed herein comprise administering an IL-27-inhibiting agent, e.g., an antibody or an antigen-binding portion thereof that specifically binds to to IL-27, and a CD112R inhibitor. In some aspects, the methods disclosed herein comprise administering an IL-27-inhibiting agent, e.g., an antibody or an antigen-binding portion thereof that specifically binds to to IL-27, and a TAM receptor inhibitor. In some aspects, the methods disclosed herein comprise administering an IL-27-inhibiting agent, e.g., an antibody or an antigen-binding portion thereof that specifically binds to to IL-27, and a STING agonist and/or a 4-1BB agonist. In some aspects, an anti-IL-27 antibody, or antigen binding portion thereof, provided by the disclosure is combined (e.g., administered in combination) with a tyrosine kinase inhibitor, an agent targeting the adenosine axis (for example a CD39 antagonist, a CD73 antagonist or a A2AR, A2BR or dual A2AR/A2BR antagonist), a CCR8 antagonist, a CTLA4 antagonist, a VEG-F inhibitor or a combination thereof.

In some aspects, the methods disclosed herein comprise administering an IL-27-inhibiting agent, e.g., an antibody or an antigen-binding portion thereof that specifically binds to to IL-27, and a cell therapy. In some aspects, the cell therapy comprises a modified immune cell therapy. In some aspects, the cell therapy comprises a chimeric antigen receptor (CAR) modified immune cell therapy, e.g., CAR T therapy. In some aspects, the cell therapy comprises an engineered T cell receptor (TCR) immune cell therapy. In some aspects, the cell therapy comprises an allogeneic tumor infiltrating lymphocyte (TIL) therapy.

### EXAMPLES

While the present disclosure has been described with reference to the specific aspects thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

### Example 1: Detection of WSX-1 in Tumor Samples

Unstained formalin-fixed paraffin-embedded (FFPE) slides were dewaxed and pretreated in Bond Epitope Retrieval 2 solution (Leica Biosystems, Buffalo Grove, IL) for 20 minutes on a Leica Bond RX auto-stainer (Leica Biosystems) following manufacturer's recommended protocol. DAKO protein block (Agilent Technologies, Santa Clara, CA) was applied on slides and incubated for 15 minutes. Monoclonal rabbit anti-human WSX-1 primary antibody (cat #ab281998, Abcam, Cambridge, MA) was diluted at 1/50 in Bond antibody diluent (cat #AR9352, Leica Biosystems), dispensed on slides, and incubated for 30 minutes at room temperature, followed by incubation with a ready for use secondary antibody (HRP conjugated goat anti-rabbit IgG polymer, Bond Refine Detection kit, cat #DS9800, Leica Biosystems) for 8 minutes at room temperature, then by peroxidase block for 5 minutes (Bond Polymer Refine Detection kit, cat #DS9800, Leica Biosystems). Signal was detected by DAB incubation for 10 minutes and DAB enhancer incubation for 10 minutes (Bond Polymer Refine Detection kit, cat #DS9800, Leica Biosystems). Slides were counter stained with hematoxylin for 10 minutes. Slides were manually dehydrated, and cover slipped with poly-Mount Xylene mounting medium (cat #24176, Polysciences, Warrington, PA).

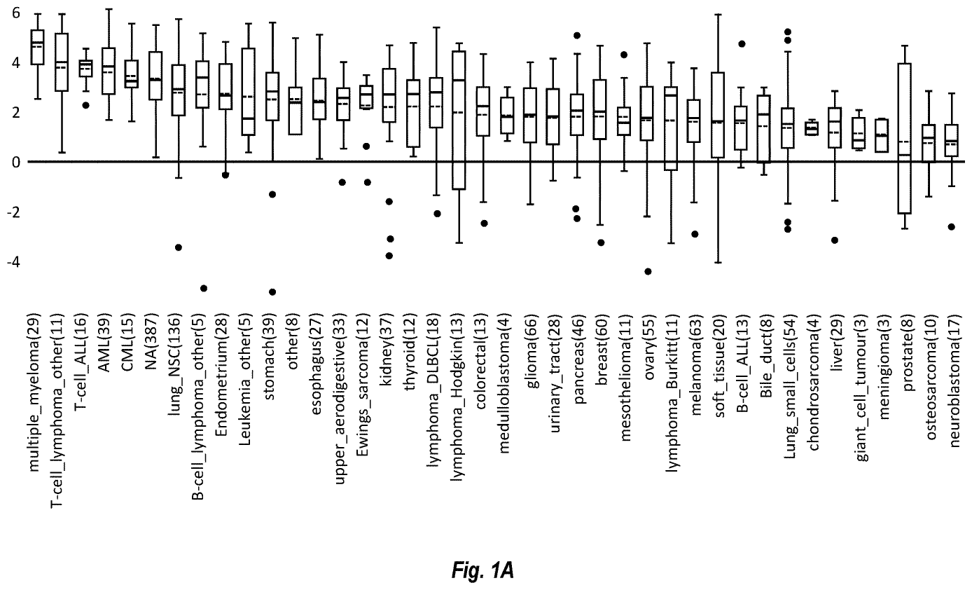

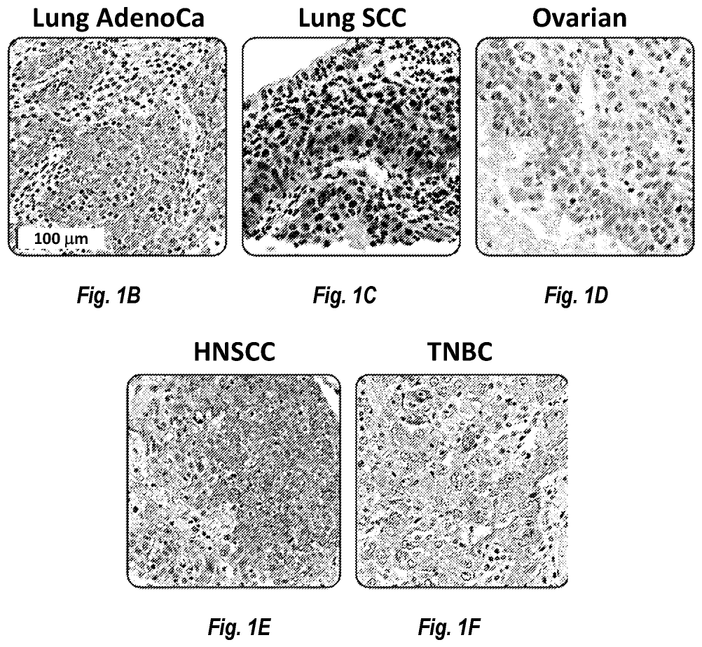

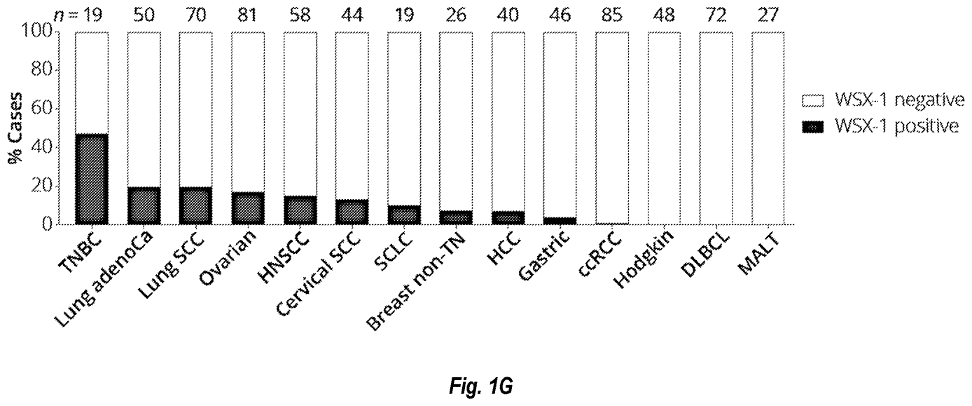

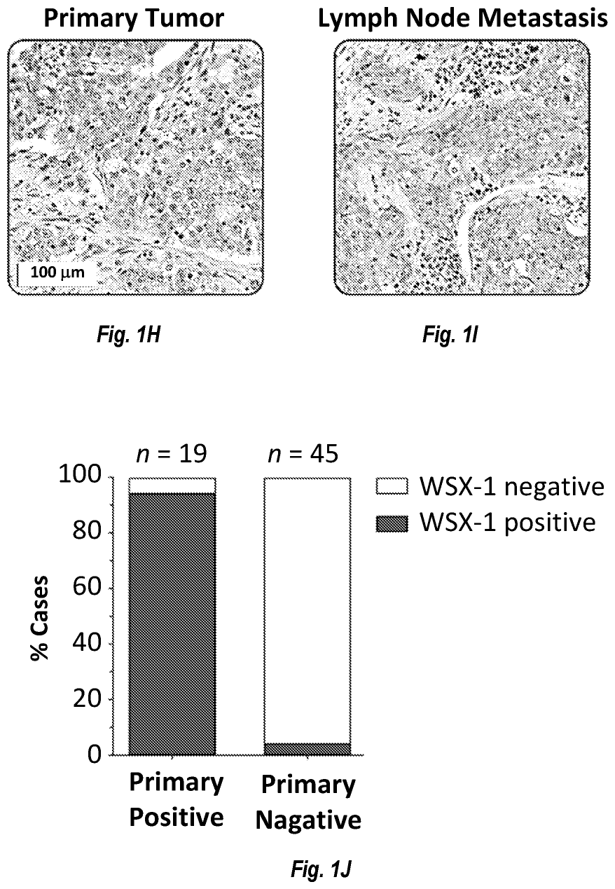

WSX-1 mRNA expression was detected in various cancer cell lines (FIG. 1A). IHC analysis of lung adenocarcinoma, lung SCC, ovarian cancer, HNSCC, and TNBC revealed positive WSX-1 expression in tumor cells in multiple indications, which is maintained at the sites of lymph node metastasis (FIGS. 1B-1J).

### Example 2: Detection of IL-27 in Tumor Samples

Unstained FFPE slides were dewaxed and pretreated in Bond Epitope Retrieval 2 solution (Leica Biosystems, Buffalo Grove, IL) for 10 minutes on a Leica Bond RX auto-stainer (Leica Biosystems) following manufacturer's recommended protocol. DAKO protein block (Agilent Technologies, Santa Clara, CA) was applied on slides and incubated for 15 minutes. Polyclonal goat anti-human IL-27 primary antibody (cat #AF2526, R&D Systems, Minneapolis, MN) was diluted to 3.33 μg/ml (1:60 dilution from a 200 μg/ml stock solution) in Bond antibody diluent (cat #AR9352, Leica Biosystems), dispensed on slides, and incubated for 60 minutes at room temperature, followed by incubation with a ready for use secondary antibody (ImmPRESS-HRP Horse anti-goat IgG polymer, cat #MP-7405, Vector Laboratories, Inc., Burlington, CA) for 8 minutes at room temperature, followed by peroxidase block for 5 minutes (Bond Polymer Refine Detection kit, cat #DS9800, Leica Biosystems). Signal was detected by DAB incubation for 10 minutes and DAB enhancer incubation for 10 minutes (Bond Polymer Refine Detection kit, cat #DS9800, Leica Biosystems). Slides were counter stained with hematoxylin for 5 minutes. The detailed Bond RX staining protocol is attached (protocol name Goat60_AZ2). Slides were manually dehydrated and cover slipped with poly-Mount Xylene mounting medium (cat #24176, Polysciences, Warrington, PA).

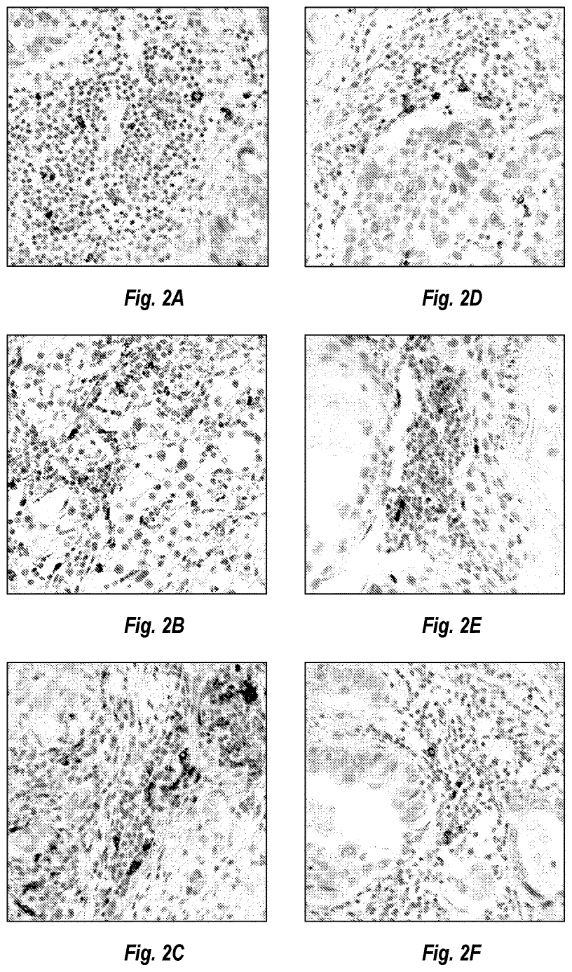

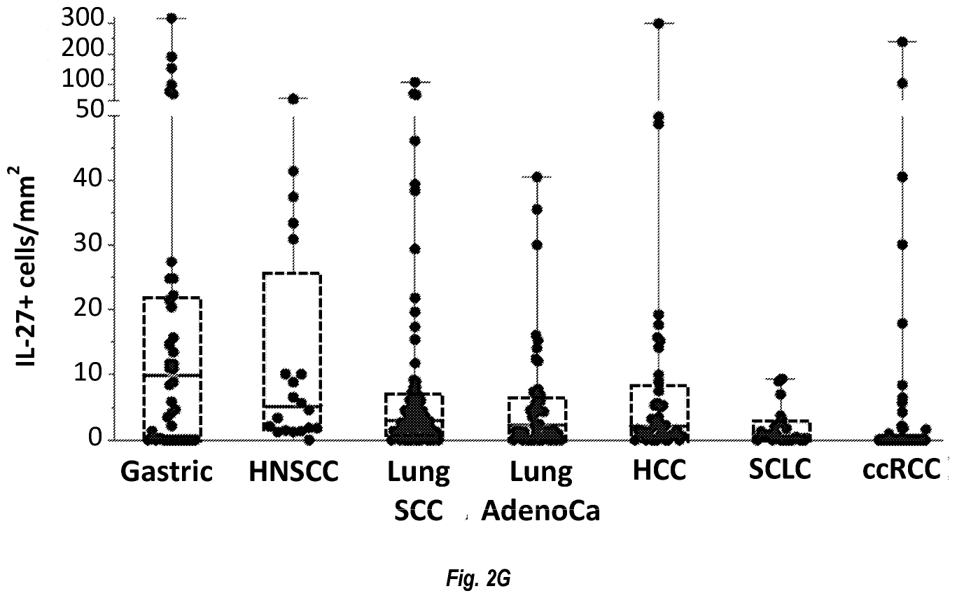

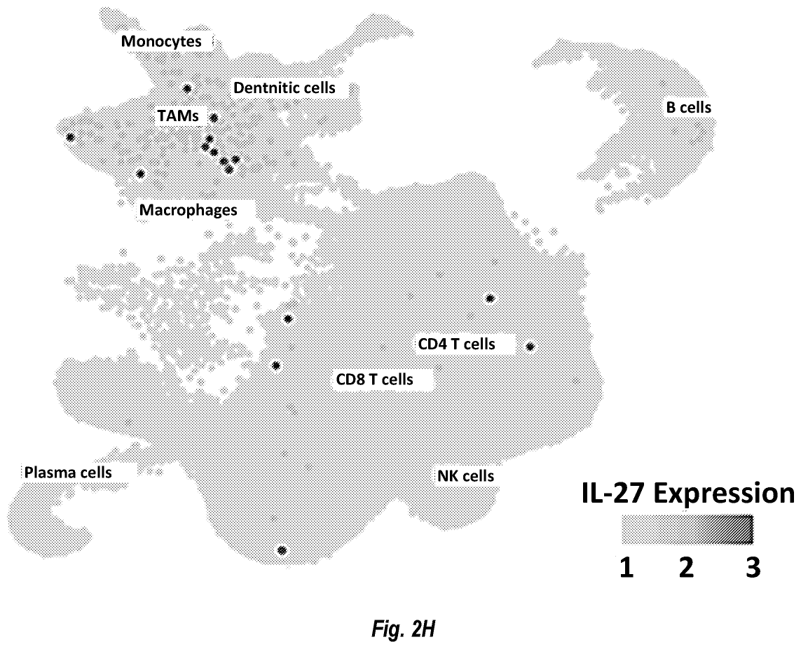

Immunohistochemistry (IHC) for IL-27 was performed on formalin-fixed, paraffin-embedded (FFPE) tumor samples in a tissue microarray (TMA) format. Staining shows positive cells in the tumor microenvironment (TME) that are morphologically consistent with tumor-associated macrophages (TAMs) across various cancer types (FIGS. 2A-2F). Quantitative image analysis on digitally scanned slides was used to quantify the density of IL-27+ cells across multiple solid tumor types (FIG. 2G). sc-RNA seq analysis of immune atlas for different cancer tumor cells for the overall IL-27 expression in all cancer types of the study is shown in FIG. 2H.

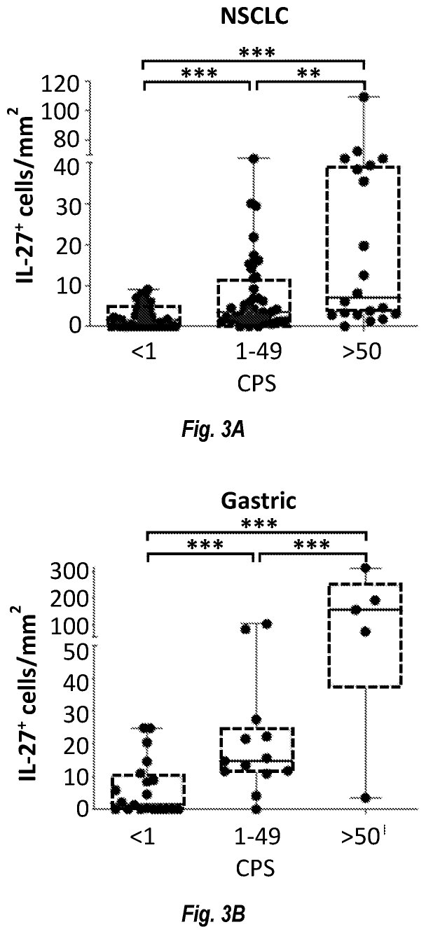

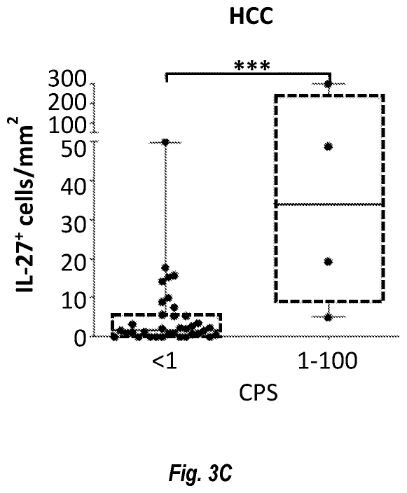

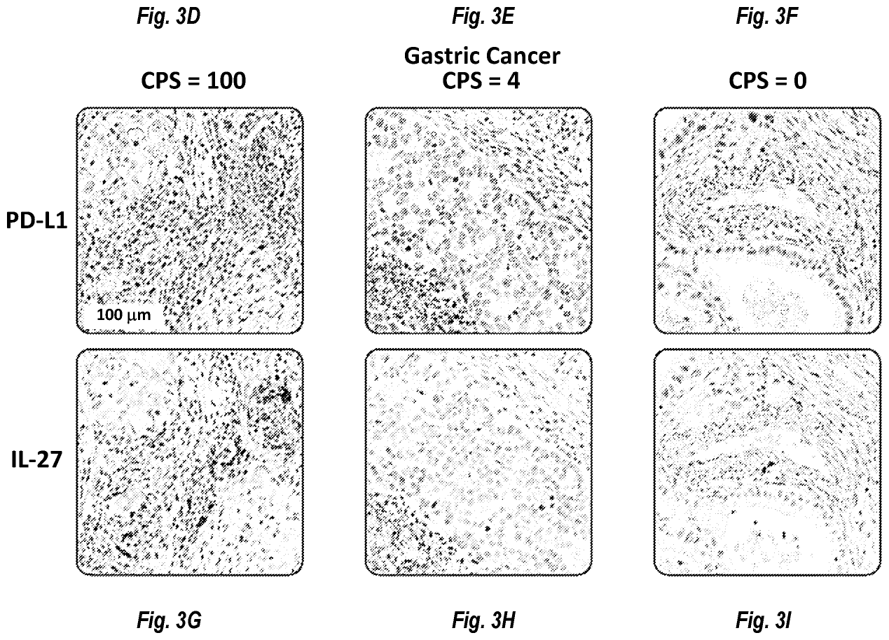

IHC for PD-L1 and IL-27 was performed on serial sections of FFPE tumor samples in TMA format. PD-L1 staining was scored semi-quantitatively using the Combined Proportional Score (CPS) system, and density of IL-27+ cells was quantified using image analysis on digitally scanned slides. In NSCLC (FIG. 3A), gastric cancer (FIG. 3B), and HCC (FIG. 3C), the density of IL-27+ cells is positively correlated with PD-L1 expression. Examples of IHC for PD-L1 and IL-27 in gastric cancer samples with a range of CPS scores are shown in FIGS. 3D-3I. CPS score correlates with the density of IL-27+ cells (FIGS. 3D, 3E, 3G, and 3H), although some tumor samples with CPS <1 (negative for PD-L1) contain IL-27+ cells (FIGS. 3F and 3I).

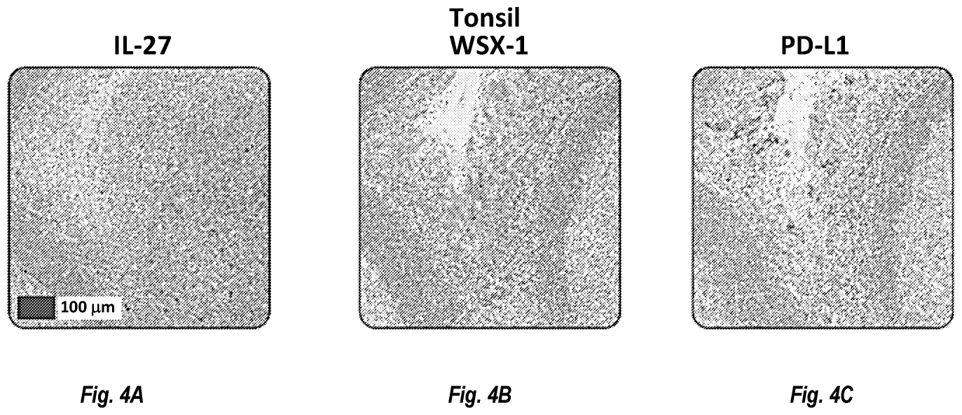

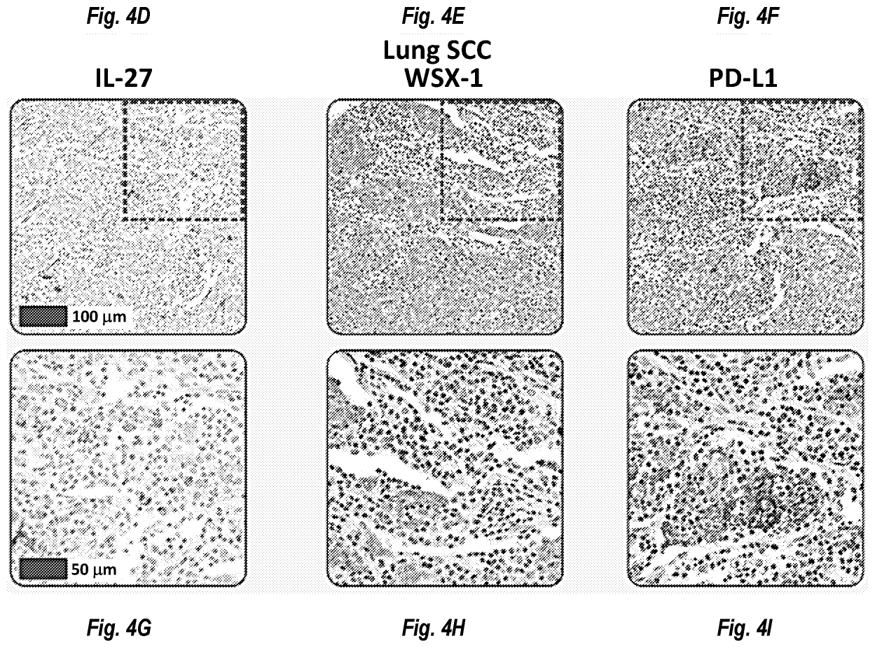

IHC for IL-27, WSX-1, and PD-L1 was performed on serial sections of a normal human tonsil and lung SCC, and positive staining for all stains was observed (FIGS. 4A-4I).

### Example 3: In Vivo Analysis of Patient Responsiveness to Anti-IL-27 Antibody Monotherapy

To identify a correlation between IL-27 expression and/or WSX-1 expression, tumor samples will be obtained from patients prior to administering an anti-IL-27 antibody therapy. Tumor samples will be assayed for expression of IL-27 and/or WSX-1 according to the methods disclosed herein, e.g., in Examples 1 and 2. Patients will be administered an anti-IL-27 antibody according to the methods disclosed herein. A retrospective analysis will be completed to identify any correlation between IL-27 expression and/or WSX-1 expression and patient overall survival, objective response rate, progression-free survival, and tumor size.

### Example 4: In Vivo Analysis of Patient Responsiveness to Anti-IL-27 Antibody Combination Therapy

To identify a correlation between IL-27 expression and/or WSX-1 expression, tumor samples will be obtained from patients prior to administering an anti-IL-27 antibody therapy. Tumor samples will be assayed for expression of IL-27 and/or WSX-1 according to the methods disclosed herein, e.g., in Examples 1 and 2. Patients will be administered (i) an anti-IL-27 antibody and (ii) an additional anticancer agent disclosed herein (e.g., an immune checkpoint inhibitor, e.g., an anti-PD-1 antibody, a chemotherapy, a radiotherapy, a cell-based immunotherapy, or any combination thereof) according to the methods disclosed herein. A retrospective analysis will be completed to identify any correlation between IL-27 expression and/or WSX-1 expression and patient overall survival, objective response rate, progression-free survival, and tumor size.

### Example 5: Detection of IL-27 and WSX-1 in NSCLC and HCC Samples

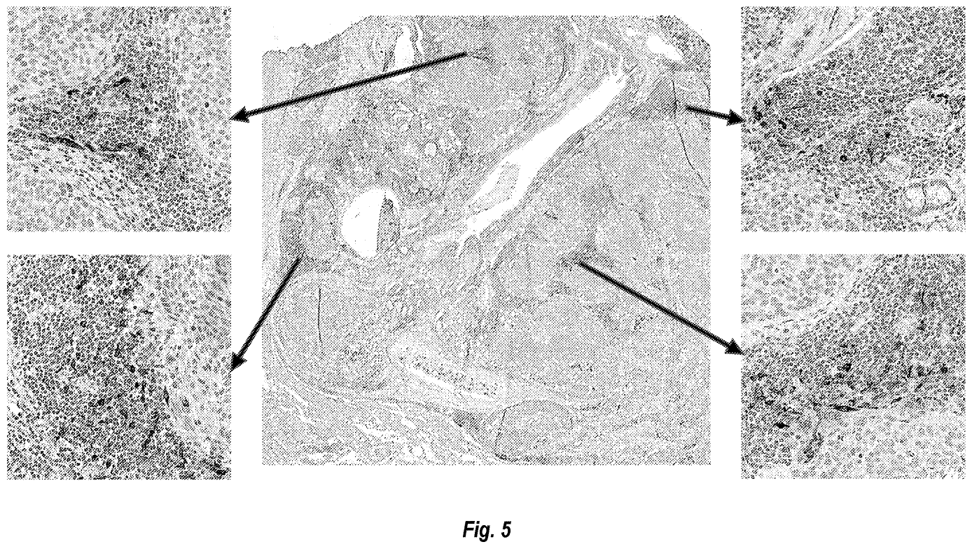

IHC for IL-27 was performed on FFPE NSCLC tumor samples obtained as whole sections from individual lobectomy specimens. IHC for IL-27 shows aggregates of IL-27+ macrophages that are unevenly distributed throughout the tumor mass (FIG. 5). Main image: 0.9× magnification; insets: 12× magnification.

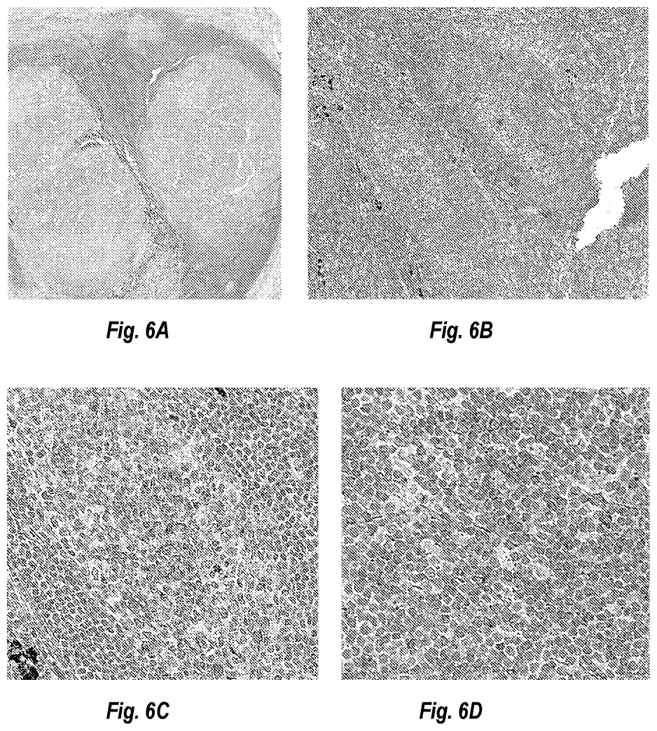

IHC for WSX-1 was performed on FFPE NSCLC tumor samples obtained as whole sections from individual lobectomy specimens. IHC for WSX-1 shows immune cell staining for WSX-1 in the peritumoral immune cell infiltrate (FIG. 6A, 0.55× magnification). WSX-1 immune cell staining is prominent in Tertiary Lymphoid Structures (TLS) (FIG. 6B, 3.5× magnification). Follicular Dendritic Cells (FDCs) within the germinal centers of TLS are positive for WSX-1 (FIG. 6C, 12× magnification). Immune cell staining for WSX-1 is also seen outside TLS (FIG. 6D, 20× magnification).

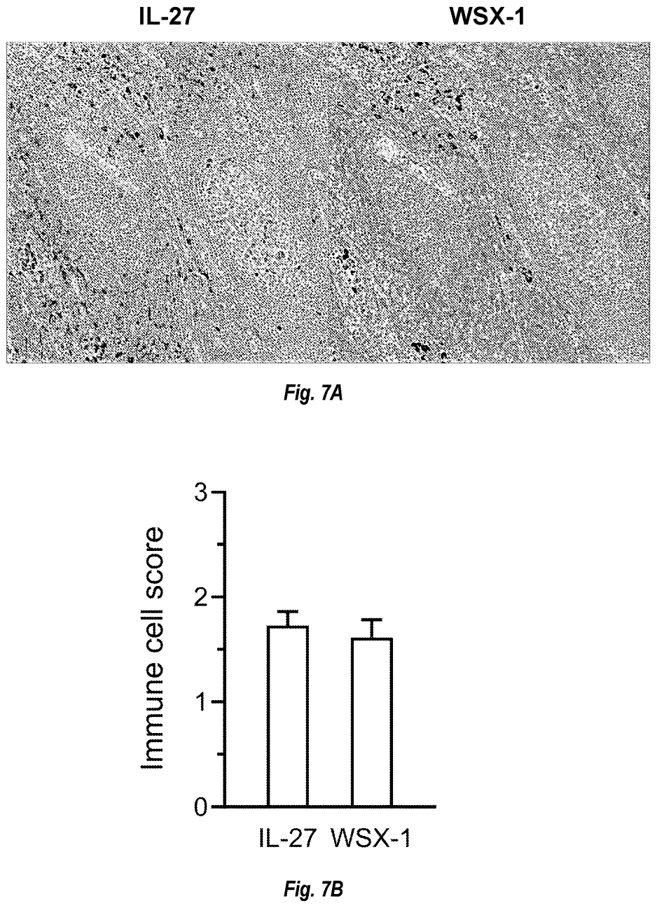

IHC for IL-27 and WSX-1 was performed on FFPE NSCLC tumor samples obtained as whole sections from individual lobectomy specimens. IHC shows immune cell staining for IL-27 and WSX-1 within the same areas in the tumor microenvironment (TME), most noticeably in the TLS (FIG. 7A, matching fields from same specimen, 7× magnification). Immune cell staining for IL-27 and WSX-1 was semi-quantitatively scored using the following pathology scoring system: 0, no staining or rare positive immune cells; 1+, positive immune cells seen in less than 33% of tumor tissue area; 2+, positive immune cells seen in 33-66% of tumor tissue area; 3+, positive immune cells seen in >66% of tumor tissue area (FIG. 7B).

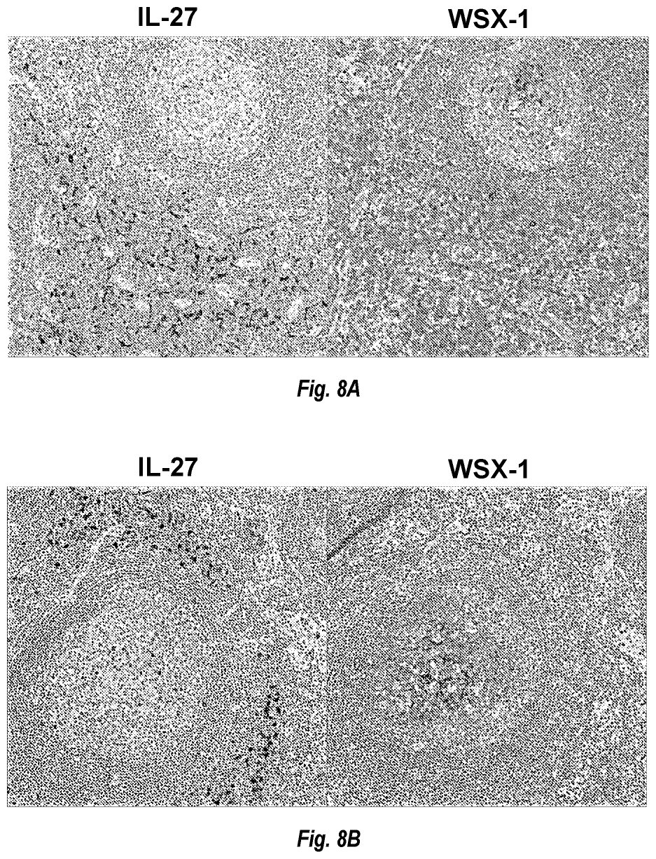

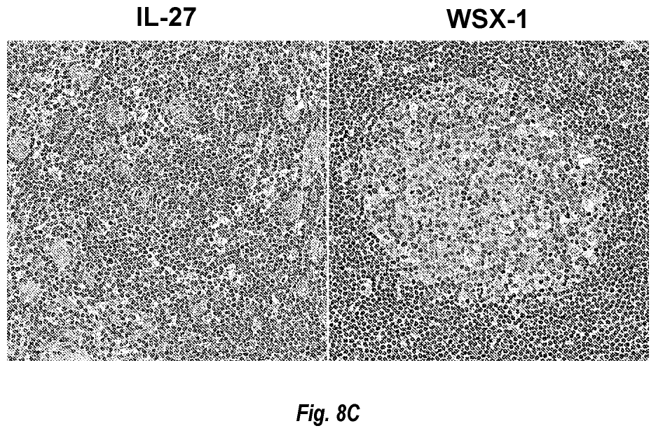

IHC for IL-27 and WSX-1 was performed on FFPE samples of NSCLC draining lymph nodes obtained from individual lobectomy specimens and showed abundant immune cell staining for both markers (FIG. 8A, 8B). Specifically, there are abundant IL-27+ macrophages within interfollicular areas, abundant WSX-1+ immune cells within interfollicular areas, and prominent WSX-1+ FDCs within the germinal centers. This is observed in draining lymph nodes that contain tumor (lymph node metastases; FIG. 8A, matching fields from same specimen, 10× magnification) as well as draining lymph nodes that do not contain tumor (FIG. 8B, matching fields from same specimen, 7× magnification). In contrast, IHC for IL-27 and WSX-1 on control lymph nodes show minimal immune cell staining for IL-27 and WSX-1 (FIG. 8C, 15× magnification). n=2-5 specimens per group (5 NSCLC draining lymph nodes with tumor, 2 NSCLC draining lymph nodes without tumor, and 5 control lymph nodes).

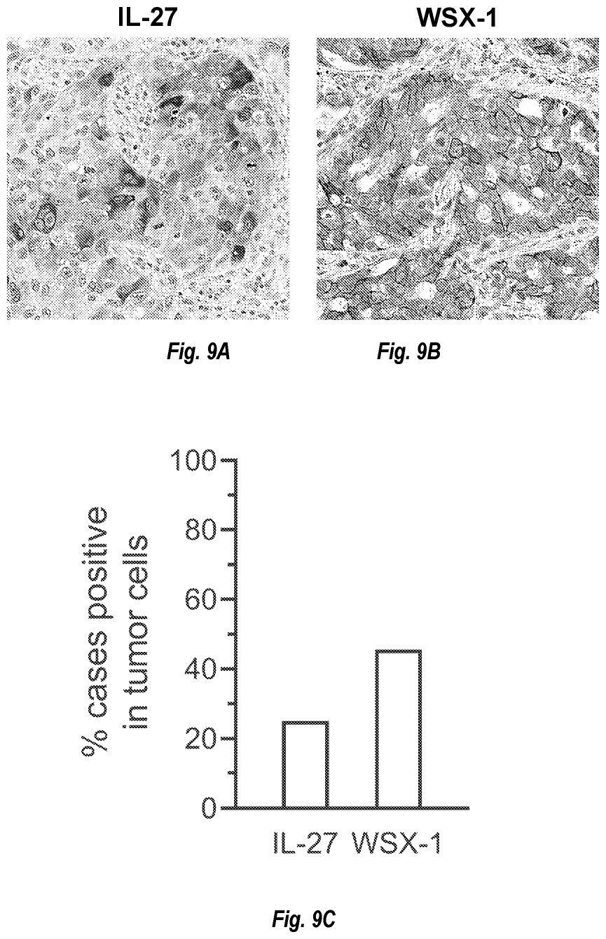

A subset of NSCLC samples shows tumor cell staining for IL-27 or WSX-1 (FIG. 9A, FIG. 9B, and FIG. 9C). Cytomembranous (cytoplasmic and/or membranous) tumor cell staining for IL-27 in at least 1% of tumor cells is seen in 25.0% of NSCLC cases (FIG. 9A, 20× magnification; FIG. 9C). Membranous tumor cell staining for WSX-1 in at least 1% of tumor cells is seen in 45.5% of NSCLC cases (FIG. 9B, 20× magnification; FIG. 9C). Bars show mean for n=44 NSCLC lobectomy specimens (FIG. 9C).

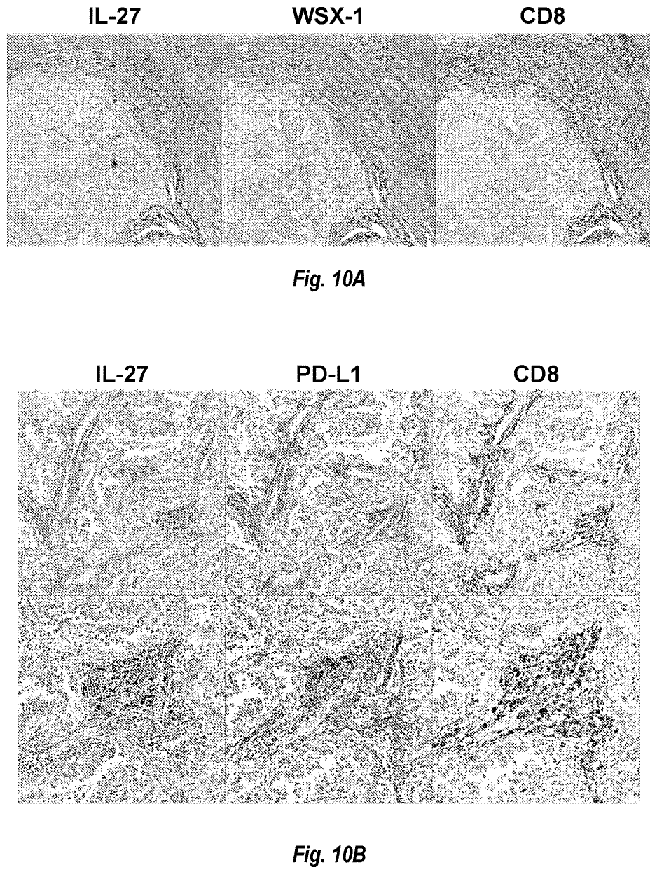

IHC for CD8 shows that, in NSCLC cases with immune-excluded pattern (where CD8+ T-cells are largely confined to the peritumoral immune cell infiltrate, with minimal penetration into the tumor mass), there is prominent immune cell staining for IL-27 and WSX-1 in the peritumoral immune cell infiltrate where CD8+ T-cells accumulate (FIG. 10A, 2.2× magnification). Similarly, in NSCLC cases with a more subtle immune-excluded pattern (where CD8+ T-cells enter the main tumor mass but are essentially confined to the stroma, without significant infiltration of tumor nests), aggregates of IL-27+ macrophages are seen in the stromal areas where CD+ T-cells accumulate (FIG. 10B, 4× magnification [top] and 10× magnification [bottom]). Immune cell staining for PD-L1 is also seen in the same stromal areas (FIG. 10B).

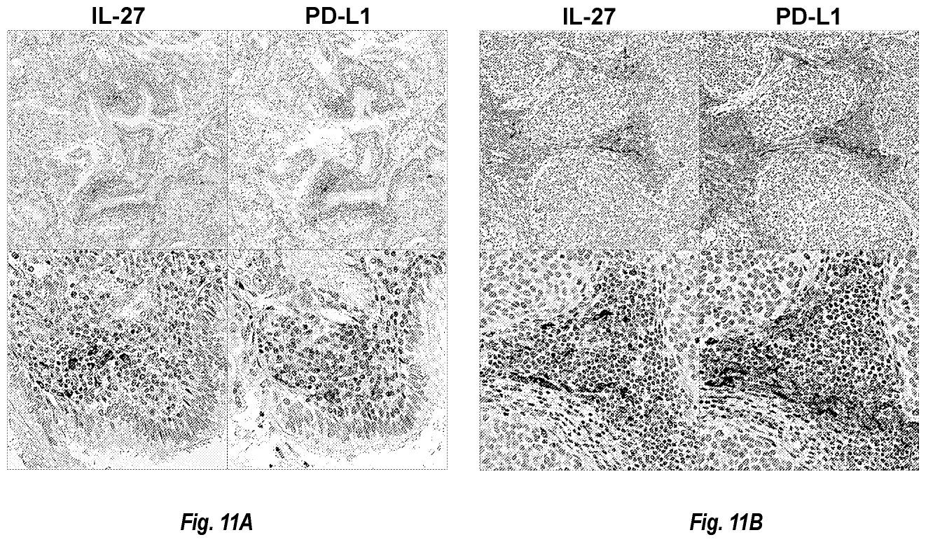

In NSCLC, immune cell staining for PD-L1 is seen within the same stromal areas that contain aggregates of IL-27+ macrophages (FIG. 11A, lung adenocarcinoma, 4× magnification [top] and 20× magnification [bottom], and FIG. 11B, lung squamous cell carcinoma, 6× magnification [top] and 18× magnification [bottom]). Within these areas, the morphology of the PD-L1+ immune cells is very similar to that of the IL-27+ macrophages, which suggests that there may be a population of macrophages in the NSCLC TME that is positive for both markers.

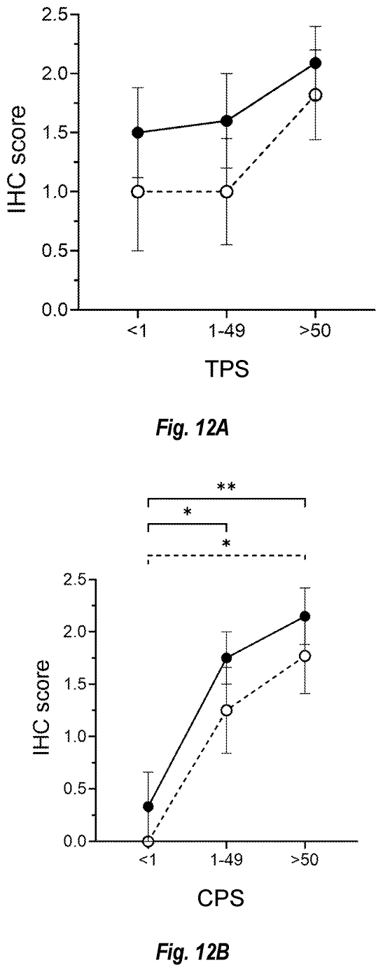

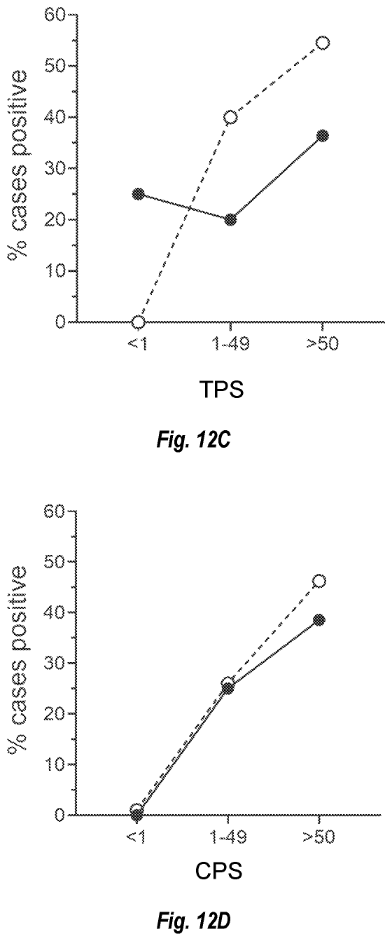

In NSCLC, expression of IL-27 and WSX-1 generally correlates with expression of PD-L1 (FIG. 12A, 12B, 12C, 12D). When PD-L1 status is classified using the Tumor Proportional Score (TPS) system, which takes into account PD-L1 expression on tumor cells only, there is a trend towards higher immune cell expression of IL-27 and WSX-1 in cases with higher PD-L1 expression (FIG. 12A). When PD-L1 status is classified using the Combined Proportional Score (CPS) system, which takes into account PD-L1 expression on tumor cells and immune cells, there is statistically significantly higher immune cell expression of IL-27 and WSX-1 in cases with higher PD-L1 expression (FIG. 12B). Data points are mean±SEM for n=24 NSCLC lobectomy specimens. Immune cell staining for IL-27 and WSX-1 (IHC score) was semi-quantitatively scored using the following pathology scoring system (as previously described in FIG. 7): 0, no staining or rare positive immune cells; 1+, positive immune cells seen in less than 33% of tumor tissue area; 2+, positive immune cells seen in 33-66% of tumor tissue area; 3+, positive immune cells seen in >66% of tumor tissue area. *: p<0.01, **: p<0.01 by Student's T-test. All other comparisons were not statistically significant. Similarly, tumor cell staining for IL-27 and WSX-1 is more frequent in cases with higher PD-L1 expression, especially when PD-L1 expression is classified using the CPS system (FIG. 12C and FIG. 12D). Data points are percentage of positive cases for n=24 NSCLC lobectomy specimens. Positivity is defined as showing cytomembranous (IL-27) or membranous (WSX-1) tumor cell staining in at least 1% of tumor cells.

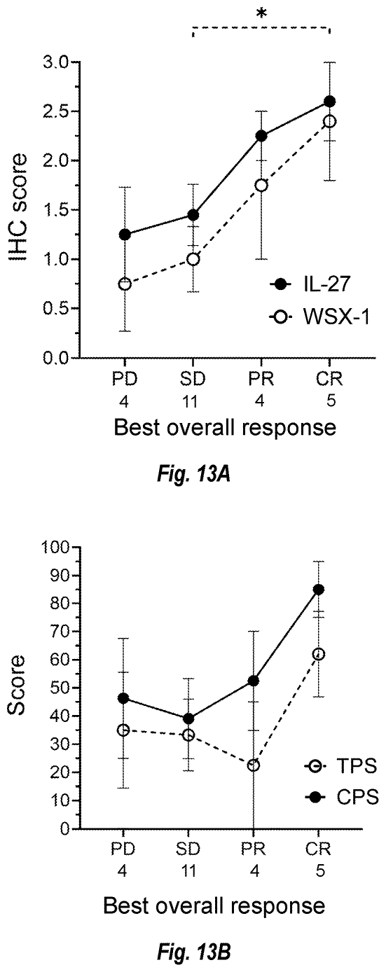

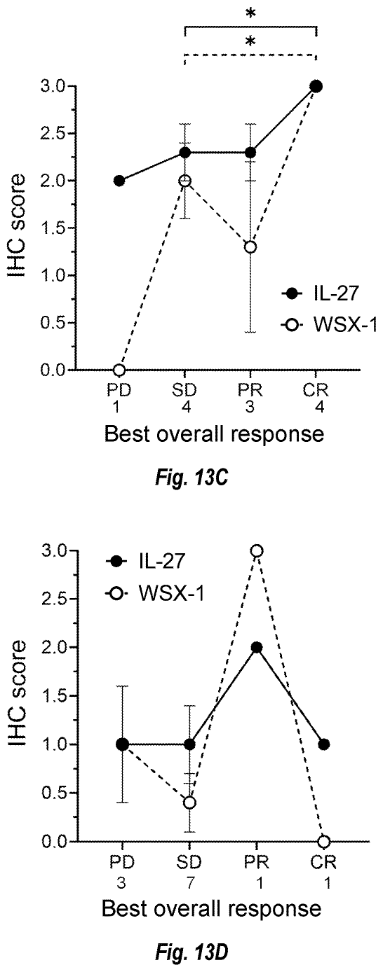

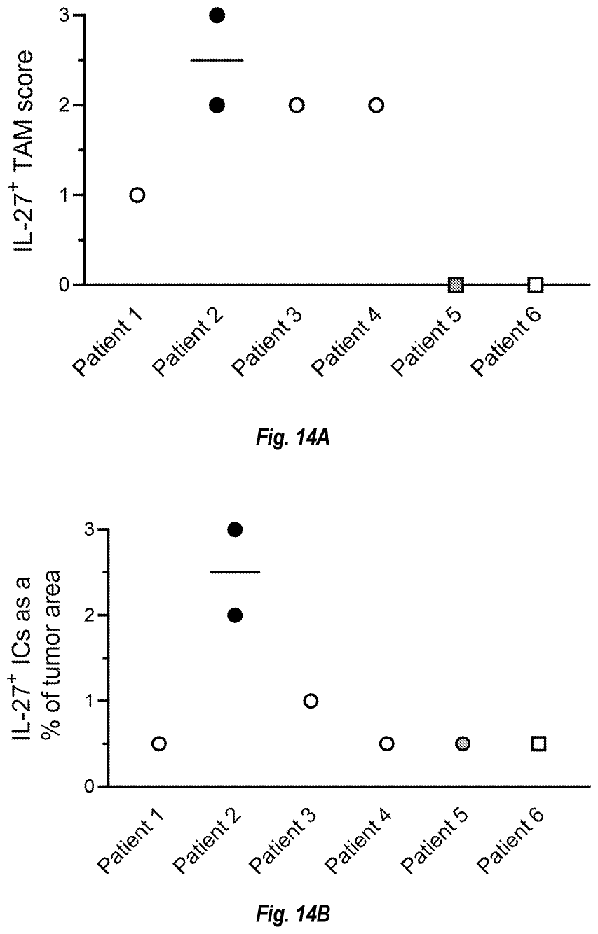

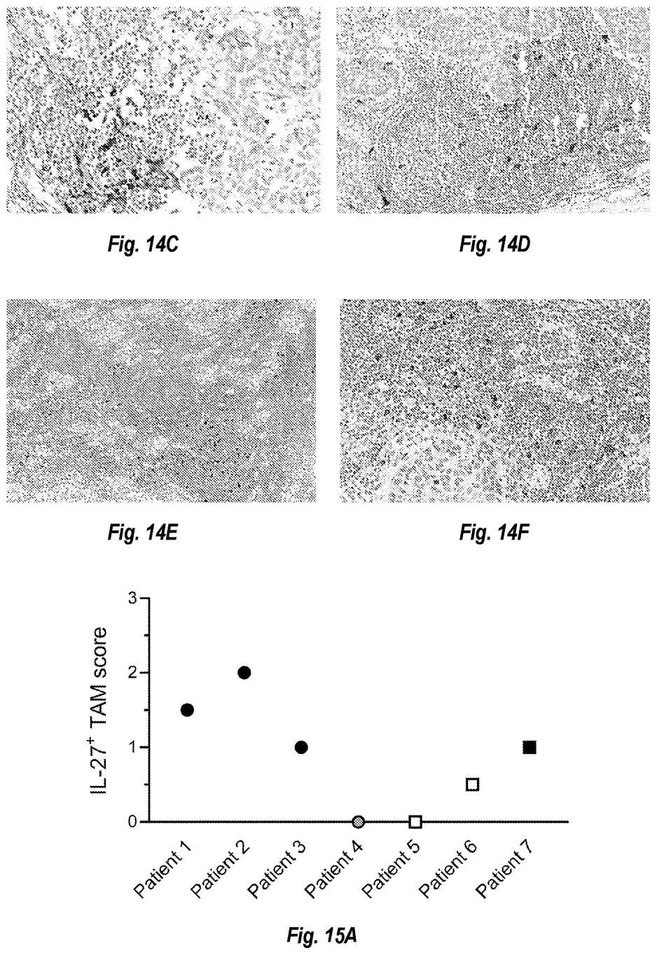

In NSCLC, there is a general trend towards higher immune cell expression of IL-27 and WSX-1 in patients who subsequently respond to immune checkpoint blockade (ICP) (FIG. 13A). There is also a general trend between response to ICP and PD-L1 status (FIG. 13B), although this trend is statistically weaker than the trend between response to ICP and immune cell expression of IL-27 and WSX-1. Data points are mean±SEM for n=24 NSCLC lobectomy specimens. IHC was performed on archival resection specimens from patients who were subsequently treated with ICP, and the best overall response was recorded. Immune cell staining for IL-27 and WSX-1 (IHC score) was semi-quantitatively scored using the following pathology scoring system (as previously described in FIG. 7 and FIG. 11): 0, no staining or rare positive immune cells; 1+, positive immune cells seen in less than 33% of tumor tissue area; 2+, positive immune cells seen in 33-66% of tumor tissue area; 3+, positive immune cells seen in >66% of tumor tissue area. TPS: Tumor Proportional Score, CPS: Combined Proportional Score. *: p<0.01 by Student's T-test. All other comparisons not statistically significant. PD: Progressive Disease, SD: Stable Disease, PR: Partial Response, CR: Complete Response. Number of patients in each response category shown below response label. All scores (TPS, CPS, IL-27 score, and WSX-1 score) were generated by a pathologist blinded to the response data. When patients are divided between those who received ICP as first-line therapy (FIG. 13C) and those who received ICP as second-line or higher therapy (FIG. 13D), the trend between response to ICP and immune cell expression of IL-27 and WSX-1 is observed only in the first-line patients, although as a caveat the number of patients in each cohort is low. Data points are mean±SEM for n=12 NSCLC lobectomy specimens in each cohort. All other details are as for FIG. 13A and FIG. 13B. IHC for IL-27 on archival specimens from NSCLC patients subsequently treated with SRF388 shows that the greatest abundance of IL-27+ macrophages is seen in specimens from a patient who exhibited a partial response to SRF388 monotherapy (FIG. 14A, FIG. 14B). This patient is the only NSCLC responder to SRF388 monotherapy for whom archival tumor samples where available for IL-27 IHC. This observation is seen when specimens are scored using the IL-27+ macrophage score by a pathologist (previously described in FIG. 7, FIG. 11, and FIG. 12) (FIG. 14A) and also when specimens are scored by a Contract Research Organization (CRO) using a more precise scoring system (positive IL-27+ immune cells as a percentage of tumor area) (FIG. 14B). The latter scoring system shows less abundant IL-27+ macrophages in patients who did not respond to SRF388 monotherapy, and thus shows more of a difference in IL-27+ macrophage abundance between archival specimens from non-responders vs. from the responder to SRF388 monotherapy. All scores were generated blinded to response data. Squares indicate archival biopsy specimens; circles indicate archival resection specimens. Black circles/squares indicate partial response to SRF388 monotherapy; gray circles/squares indicate stable disease; white circles/squares indicate progressive disease. Note that two archival specimens were available for Patient 2 (a lung resection specimen and a lymph node metastasis resection specimen); the line for Patient 2 indicates the mean of the two specimens. For Patient 2, IHC for IL-27 on the archival lung resection specimen showed numerous aggregates of IL-27+ macrophages in the TME (FIG. 14C, 20× magnification), including adjacent to Tertiary Lymphoid Structures (TLS) (FIG. 14D, 15× magnification, asterisk indicates TLS). IHC for IL-27 on the archival lymph node metastasis specimen of Patient 2 showed abundant IL-27+ macrophages in the residual lymph node tissue (FIG. 14E, 8× magnification, and FIG. 14F, 20× magnification).

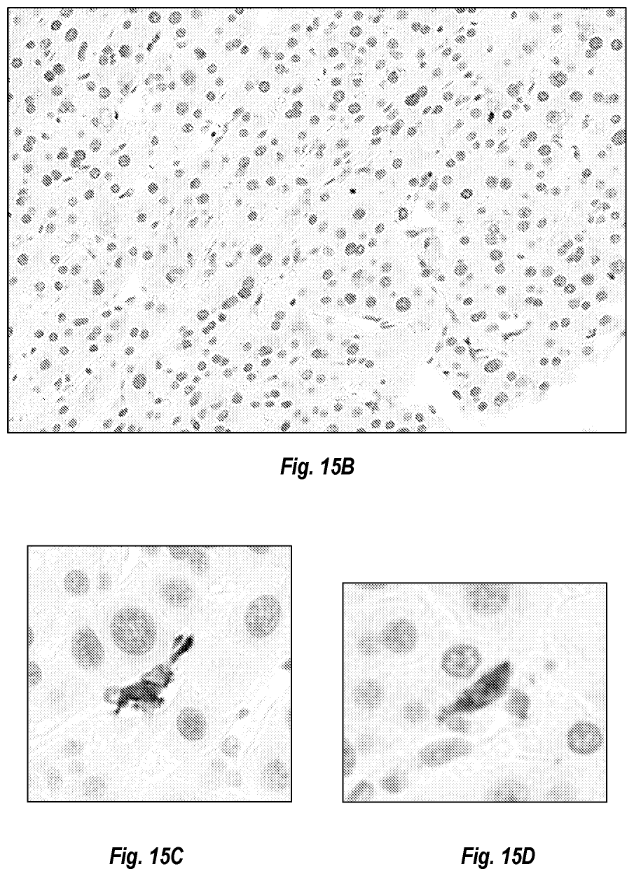

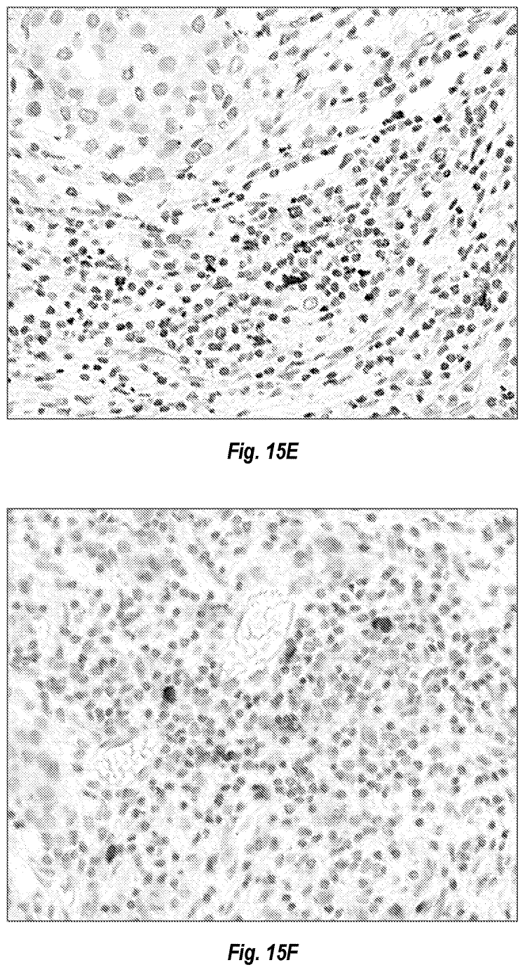

IHC for IL-27 on archival specimens from hepatocellular carcinoma (HCC) patients subsequently treated with SRF388 in combination with atezolizumab and bevacizumab shows that IL-27+ macrophages are present in the TME of all patients who responded to the triple combination therapy (among patients for whom archival specimens were available for IL-27 IHC) (FIG. 15A). The IL-27+ macrophage score was generated by a pathologist using the same scoring system as previously described in FIG. 7, FIG. 11, FIG. 12, and FIG. 13, blinded to response data. Scores using the more precise scoring system generated by the CRO are pending. Squares indicate archival biopsy specimens; circles indicate archival resection specimens. Black circles/squares indicate partial or complete response to SRF388 therapy in combination with atezolizumab and bevacizumab; gray circles/squares indicate stable disease; white circles/squares indicate progressive disease. IHC for IL-27 on patients who responded to SRF388 combination therapy typically showed IL-27+ macrophages infiltrating the tumor mass in a single-cell fashion, and located within sinusoidal-like spaces in close apposition to tumor cells; this pattern is reminiscent of the sinusoidal location of Kupffer cells (tissue-resident macrophages of the liver) in benign liver tissue (FIG. 15B, 20× magnification, and FIG. 15C and FIG. 15D, insets focusing on IL-27+ macrophages). However, IL-27 IHC in one case (Patient 2) showed distinct aggregates of IL-27+ macrophages within the peritumoral or stromal immune cell infiltrate, very similar to the pattern of IL-27 IHC seen in NSCLC specimens and described above (FIG. 15E and FIG. 15F, 20× magnification).

### Example 6: Identifying IL-27 Dependent Biomarkers in Lymphocytes, NK Cells and Myeloid cells in Peripheral Blood and the Tumor Microenvironment

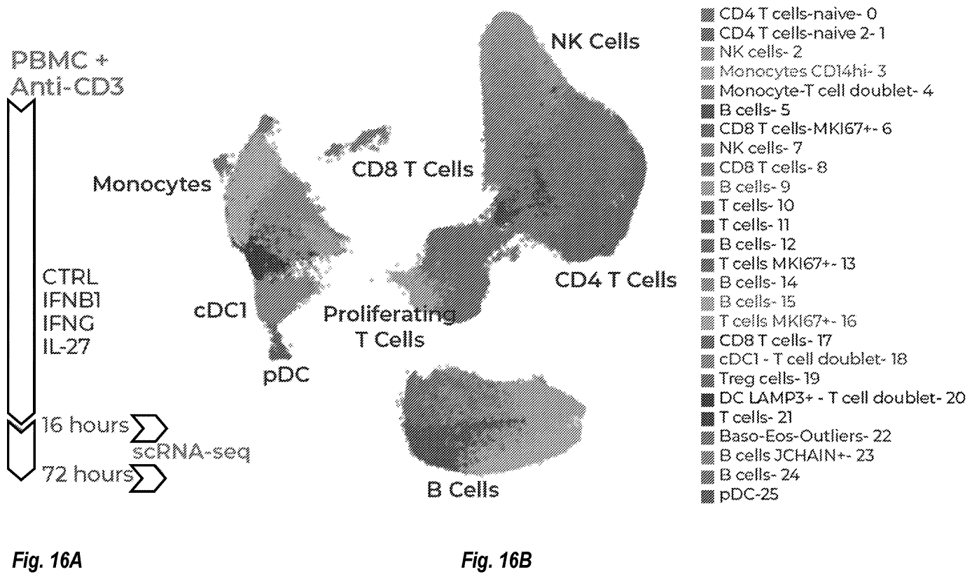

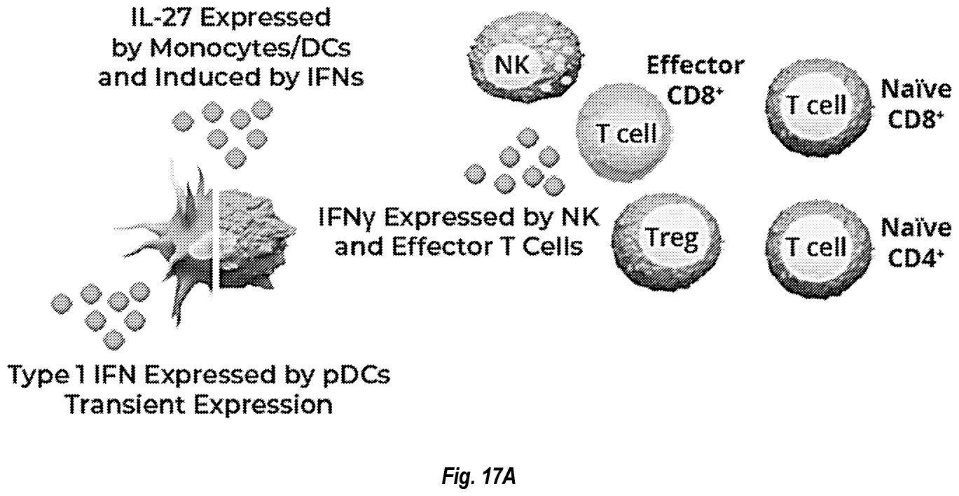

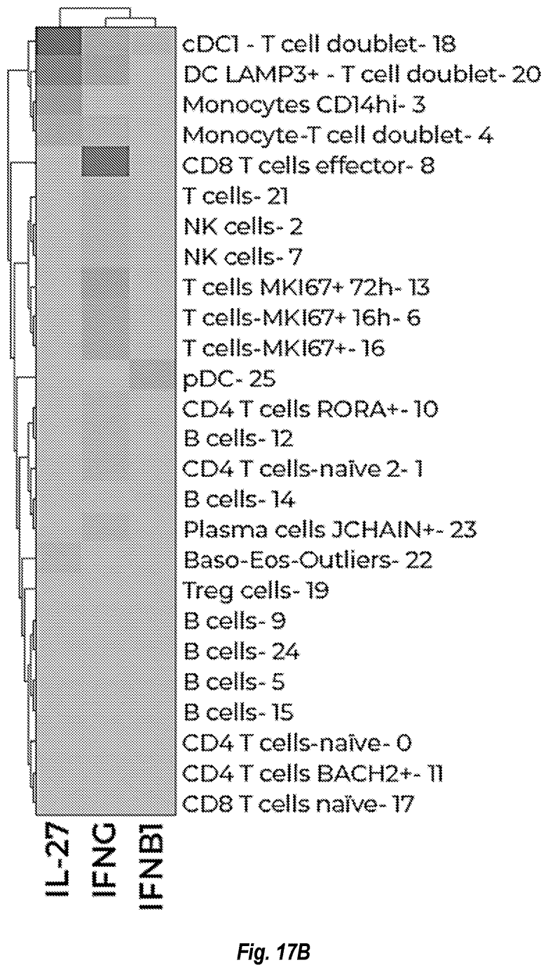

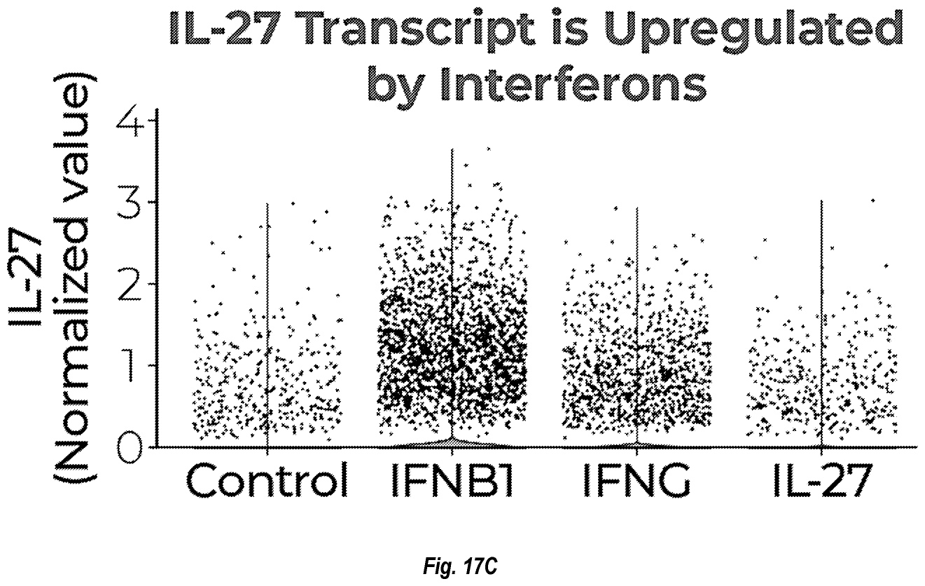

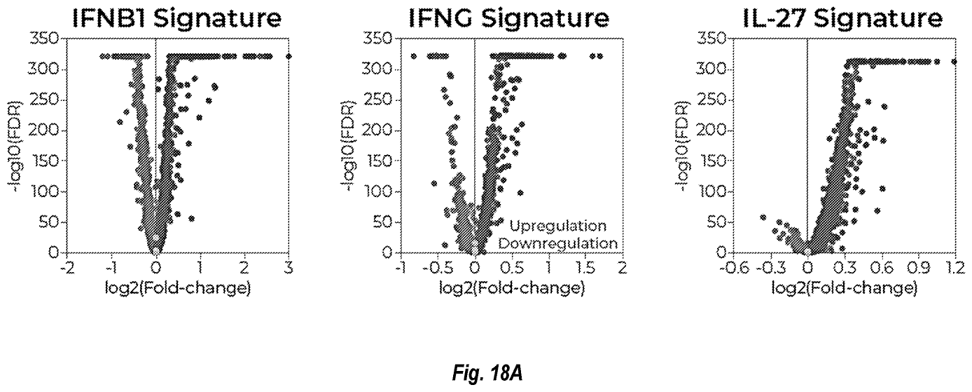

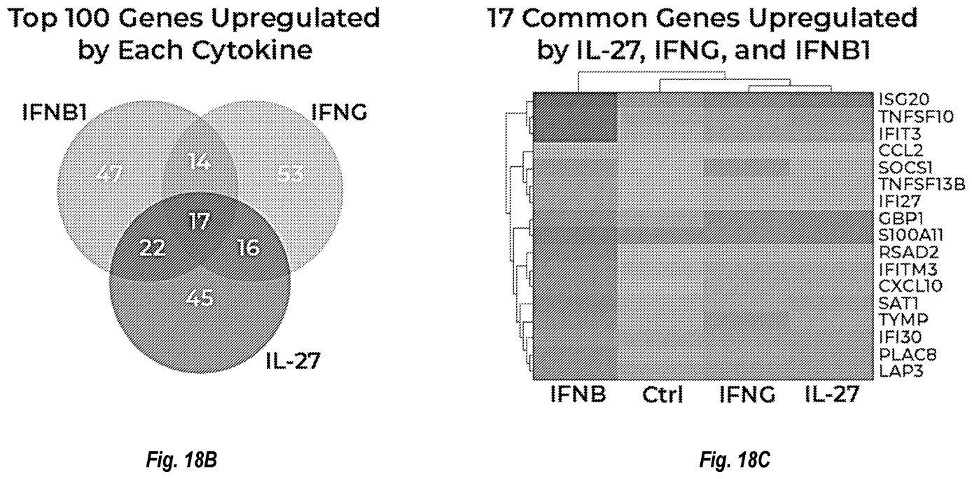

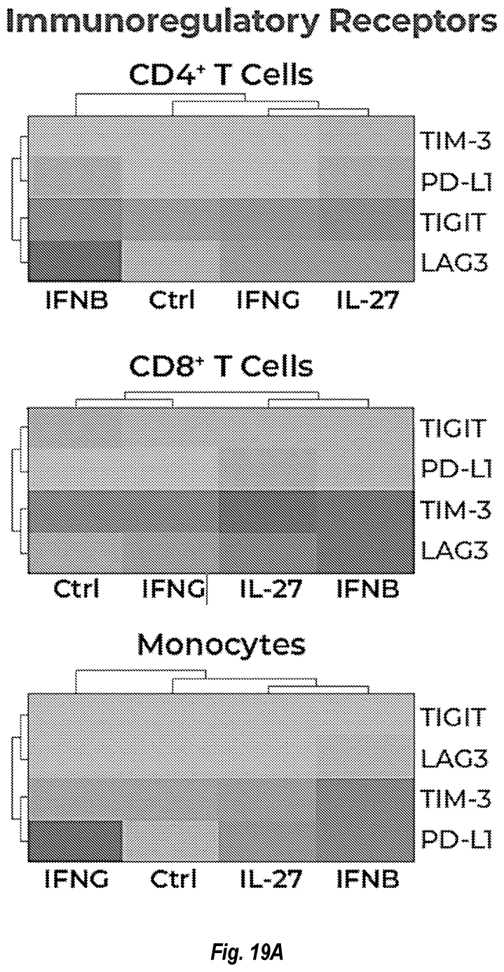

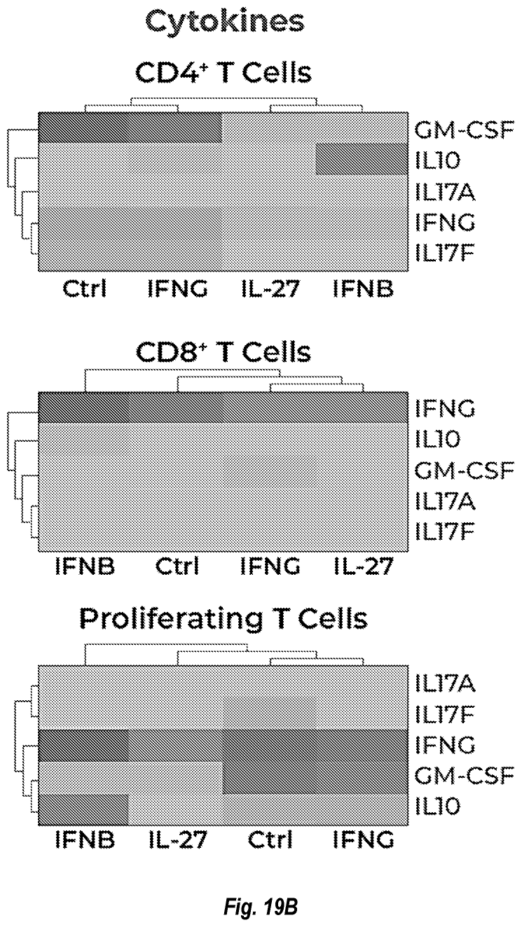

Human PBMCs treated with IL-27, interferons (IFNs), or the STING pathway agonist cGAMP, were analyzed by single cell RNA sequencing (FIG. 16A). Pooled human PBMC from healthy donors were stimulated with anti-CD3 (0.25 μg/mL) in vitro in the presence/absence of recombinant human rhIL-27, rhIFNA2, rhIFNB1, or rhIFNG (all 100 ng/ml). After 16 and 72 hr, cells were processed for scRNA-seq (10× Genomics). Seurat-based clustering was used to identify cell subsets that were assigned based on differential gene expression. UMAP representations of the aggregated data from all conditions identifying different cell populations. Immunohistochemistry (IHC) was used to assess IL-27 and its receptor WSX-1 (IL-27RA), PD-L1, and GBP5 in human tumors (FIG. 16B). As shown in FIGS. 17A-C, IL-27 and Interferons are expressed in different immune cell types from activated PBMCs and interferons can upregulate IL-27 expression. Immune cell subpopulations were found to differentially express and respond to IL-27 and IFNs: pDC transiently express IFNα/IFNβ, T and NK cells produce IFNγ, and myeloid cells express IL-27 (FIG. 17B). IL-27 and interferons also upregulate the expression of several canonical interferon stimulated genes (FIGS. 18A-18C). Additionally, IL-27 and interferons were shown to commonly upregulate the expression of checkpoint receptors in different cell types (FIG. 19A) but have unique properties for altering cytokine expression (FIG. 19B).

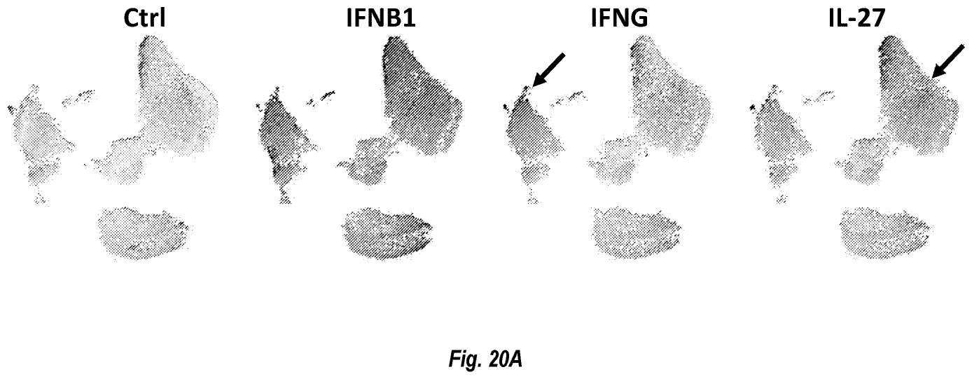

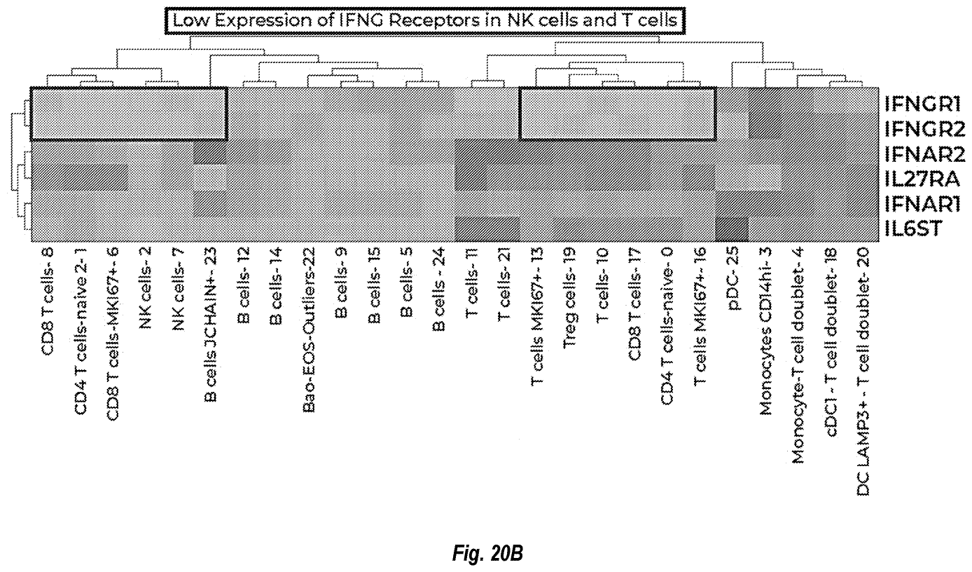

Further genes commonly upregulated by IL-27 and interferons show high expression in different cell types after stimulation with individual cytokines (FIG. 20A and FIG. 20B).

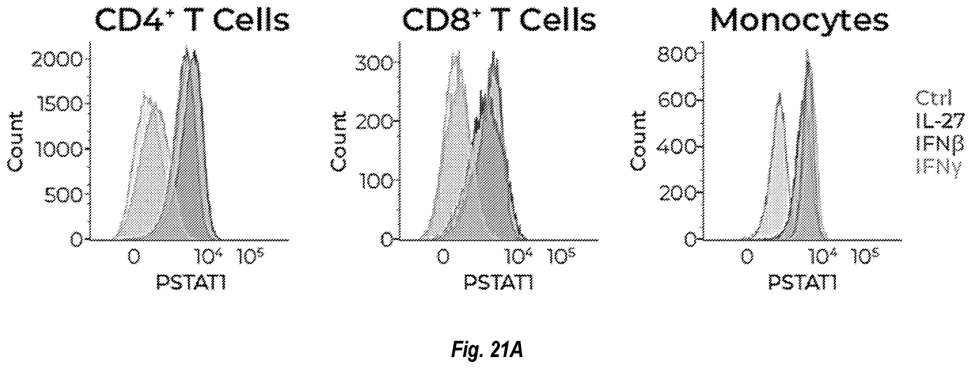

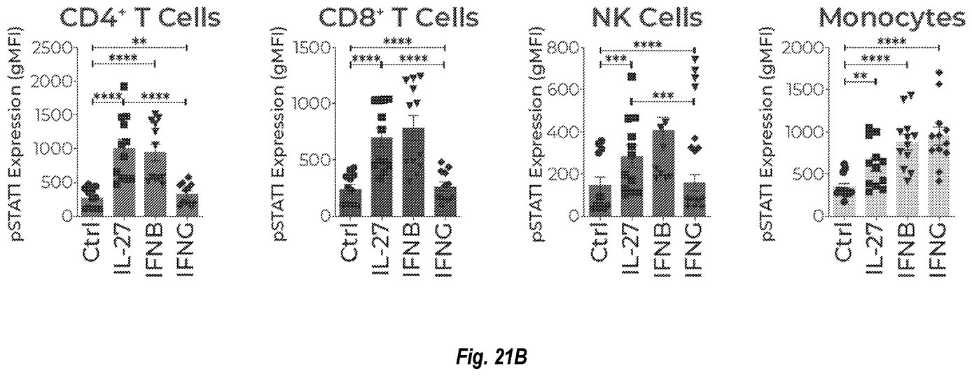

STAT1 phosphorylation was then measured in human PBMC by flow cytometry after incubation for 30 min with the indicated cytokines IL-27 and Type 1 IFN induced prominent phosphorylation of STAT1 in lymphocytes and NK cells, but Type 2 IFN did not. While IFNα/IFNβ signaling through phosphorylation of STAT1 and STAT3 was evident in all immune cells, IL-27 signaling was more restricted to T and NK cells, and IFNγ induced signaling was observed predominantly in myeloid cells (FIGS. 21A-21C).

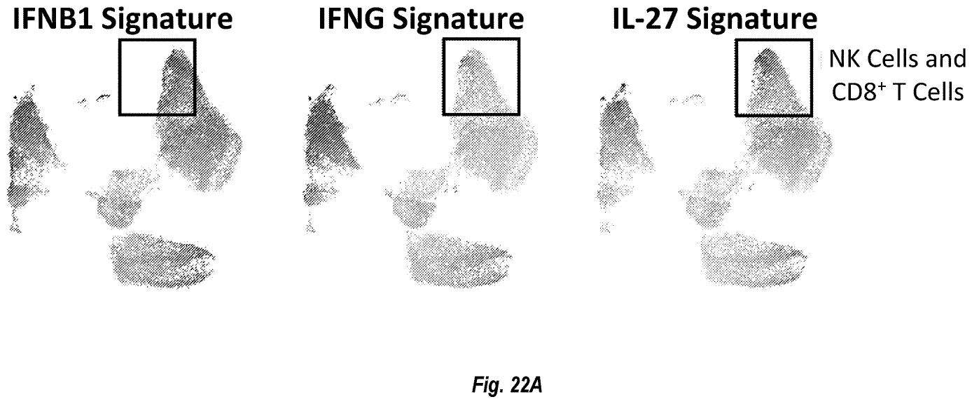

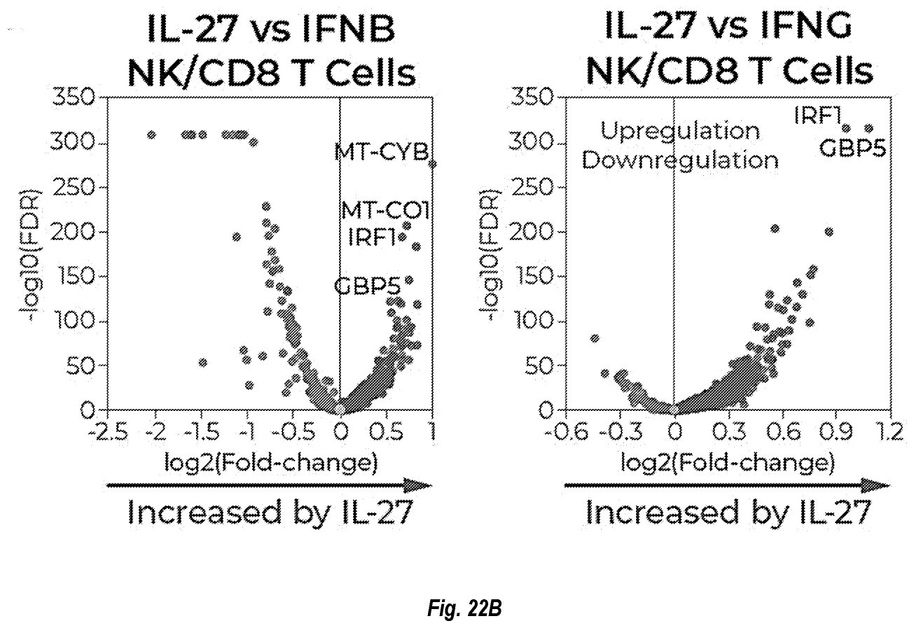

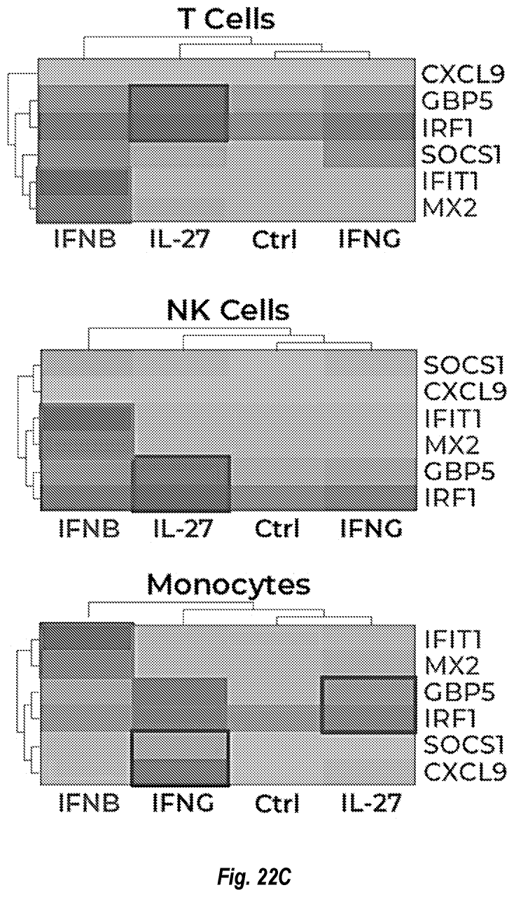

Although many canonical IFN-responsive genes were induced by both IFNs and IL-27, biased gene expression signatures were enriched in different cell types (FIGS. 22A-22C). For example, IL-27 stimulation led to differential expression of GBP5 and IRF1 in T and NK cells, IFNβ led to IFIT1 and MX2 expression in T cells, NK cells, and monocytes, while IFNγ led to SOCS1 and CXCL9 upregulation in monocytes (FIG. 22C). IL-27 and IFNo/IFNβ share a similar ability to inhibit pro-inflammatory cytokine secretion and increase expression of PD-L1 on T cells; functions that are not evident with IFNγ stimulation. Interestingly, although GBP5 and IRF1 are predominantly elevated by IL-27 in T/NK cells, these genes exhibit augmentation by IFNγ in myeloid cells.

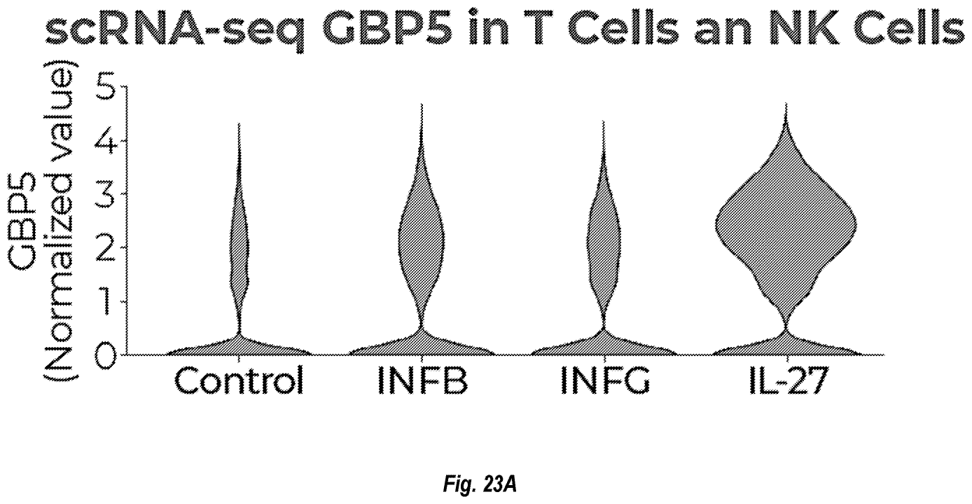

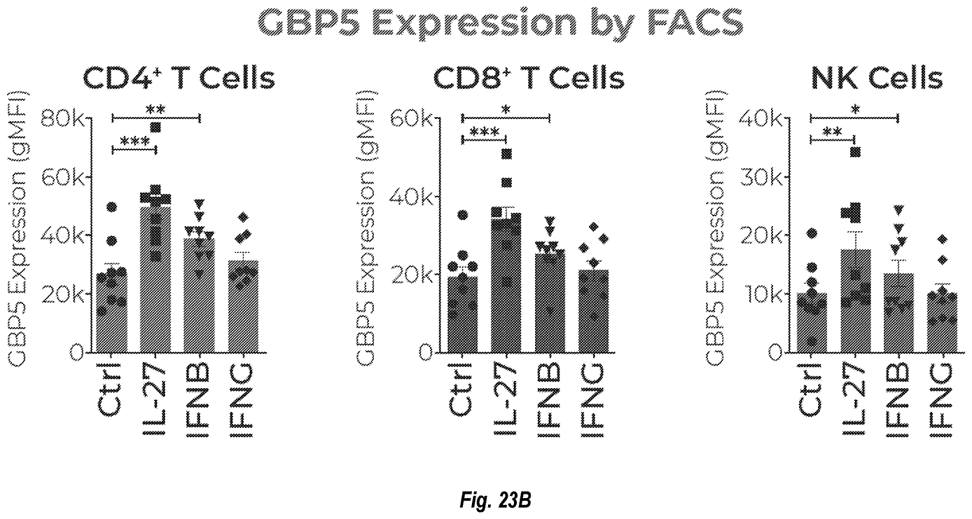

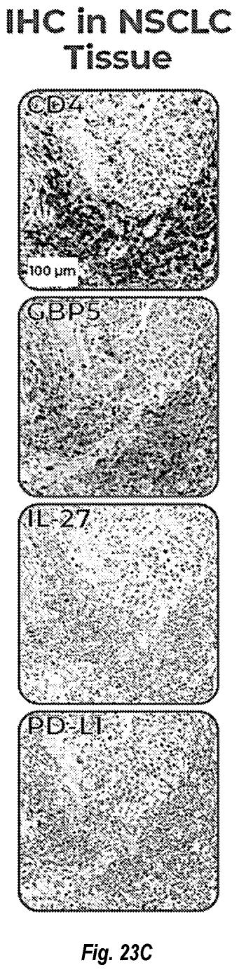

GBP5 transcript expression in T cells and NK cells after stimulation with IL-27 or Type 1 or Type 2 IFNs was analyzed (FIG. 23A). Additionally flow cytometry analysis of intracellular GBP5 expression was performed in different cell types after stimulating PBMC with the indicated cytokines for 24 hr, showing differential expression when sorted by FACs between CD4+, CD8+ or NK cells (FIG. 23B). These data indicate that IL-27 upregulates GBP5 expression in T cells and NK Cells ex vivo.

Finally, IHC on NSCLC patient samples shows that IL-27+ macrophages are co-localized with GBP5+ T-cell-rich areas in the TME along with PD-L1+ immune cells, suggestive of IL-27-dependent signaling in the NSCLC TME (FIG. 23C).

These studies highlight the complexities, redundancies and unique properties of interferons and IL-27 signaling across different immune cells. In contrast to IFNγ, IL-27 shares with type 1 interferons the ability to signal in T cells and NK cells.

1. A method for treating a subject having a WSX-1-positive tumor comprising administering to the subject an antibody, or an antigen-binding portion thereof, that specifically binds to human IL-27 and reduces or blocks the interaction between IL-27 and WSX-1, wherein the antibody or the antigen binding portion thereof comprises a heavy chain variable region comprising the amino acid sequence set forth in SEQ ID NO: 125 and a light chain variable region comprising the amino acid sequence set forth in SEQ ID NO: 133.

2. (canceled)

3. The method of claim 1, wherein the WSX-1-positive tumor is identified by detecting WSX-1 expression in a tumor sample obtained from the subject.

4. (canceled)

5. (canceled)

6. (canceled)

7. (canceled)

8. (canceled)

9. The method of claim 3, wherein at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, or at least about 50% of cells in the tumor sample express WSX-1.

10. (canceled)

11. The method of claim 3, wherein the WSX-1 expression is detected using an immunohistochemical (IHC) assay.

12. (canceled)

13. The method of claim 3, wherein the WSX-1 expression is scored using a tumor proportion score (TPS) and/or a combined positive score (CPS), wherein the TPS or CPS is at least 10%.

14. (canceled)

15. The method of claim 3, wherein the WSX-1 expression is detected by contacting the tumor sample with an antibody or an antigen-binding portion thereof that specifically binds human WSX-1.

16. A method for treating a subject having a tumor wherein one or more immune cells in a tumor sample obtained from the subject express IL-27 comprising administering to the subject an antibody, or an antigen-binding portion thereof, that specifically binds to human IL-27 and reduces or blocks the interaction between IL-27 and WSX-1, wherein the antibody or the antigen binding portion thereof comprises a heavy chain variable region comprising the amino acid sequence set forth in SEQ ID NO: 125 and a light chain variable region comprising the amino acid sequence set forth in SEQ ID NO: 133.

17. (canceled)

18. (canceled)

19. (canceled)

20. (canceled)

21. (canceled)

22. (canceled)

23. The method of claim 16, wherein the one or more immune cells in the tumor sample comprise macrophages.

24. The method of claim 16, wherein at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, or at least about 50% of the immune cells in the tumor sample express IL-27.

25. (canceled)

26. The method of claim 16, wherein the IL-27 expression is detected using an immunohistochemical (IHC) assay.

27. (canceled)

28. The method of claim 16, wherein the IL-27 expression is assessed using a tumor proportion score (TPS) and/or a combined positive score (CPS), wherein the TPS or CPS is at least 10%.

29. (canceled)

30. The method of claim 16, wherein the IL-27 expression is detected by contacting the tumor sample with an antibody or an antigen-binding portion thereof that specifically binds human IL-27.

31-63. (canceled)

64. The method of claim 1, wherein the antibody or the antigen binding portion thereof comprises a heavy chain comprising the amino acid sequence set forth in SEQ ID NO: 139 or 135 and a light chain comprising the amino acid sequence set forth in SEQ ID NO: 137.

65. (canceled)

66. (canceled)

67. The method of claim 16, wherein the antibody or the antigen binding portion thereof comprises a heavy chain comprising the amino acid sequence set forth in SEQ ID NO: 139 and a light chain comprising the amino acid sequence set forth in SEQ ID NO: 137.

68. The method of claim 1, wherein the subject has lung cancer, sarcoma, testicular cancer, ovarian cancer, pancreas cancer, breast cancer, melanoma, head and neck cancer, colorectal cancer, bladder cancer, endometrial cancer, prostate cancer, thyroid cancer, hepatocellular carcinoma (HCC), gastric cancer, brain cancer, lymphoma, leukemia, renal cancer, or a combination thereof.

69. The method of claim 1, further comprising administering an additional therapeutic agent to the subject.

70. (canceled)

71. (canceled)

72. The method of claim 69, wherein the additional therapeutic agent comprises a PD-1 antagonist, a PD-L1 inhibitor, a TIM-3 inhibitor, a LAG-3 inhibitor, a TIGIT inhibitor, a CD112R inhibitor, a TAM inhibitor, a STING agonist, a 4-1BB agonist, a multityrosine kinase inhibitor (e.g., a VEGFR inhibitor), an anti-VEGF blocking antibody, a CTLA-4 antagonist, a HIF2 antagonist, a TGFb antagonist, an mTOR inhibitor, an adenosine pathway inhibitor (e.g., an anti-CD73 antibody, an anti-CD39 antibody, an anti-A2AR antibody, an anti-A2BR, or any combination thereof), an anti-CCR8 antibody, a cytokine-based regimen (e.g., IL-2 or IFN-a), a PARP inhibitor, or a combination thereof.

73. The method of claim 69, wherein the additional therapeutic agent comprises a PD-1 antagonist.

74-87. (canceled)

88. The method of claim 1, comprising administering the antibody or antigen binding portion thereof at a dose of about 10 mg/kg to about 20 mg/kg.

89. The method of claim 16, comprising administering the antibody or antigen binding portion thereof at a dose of about 10 mg/kg to about 20 mg/kg.
